(19) United States
(12) Patent Application Publication
(10) Pub. No.: US 2026/0308513 A1
(43) Pub. Date: Oct. 8, 2026

(54) CHIMERIC ORGANOIDS

(71) Applicant: President and Fellows of Harvard College, Cambridge, MA (US)

(72) Inventors: Paola Arlotta, Cambridge, MA (US); Irene Faravelli, Cambridge, MA (US); Noelia Anton-Bolanos, Cambridge, MA (US)

(21) Appl. No.: 19/136,736

(22) PCT Filed: Dec. 6, 2023
(86) PCT No.: PCT/US2023/082779

§ 371 Date: Jun. 6, 2025

Related U.S. Application Data
(60) Provisional application No. 63/430,660, filed on Dec. 6, 2022.
(60) Provisional application No. 63/431,018, filed on Dec. 7, 2022.
(60) Provisional application No. 63/542,762, filed on Oct. 5, 2023.

(51) Int. Cl. C12N 5/079; C12N 5/00; C12N 5/073

(52) U.S. Cl. CPC C12N 5/0618; C12N 5/0603; C12N 5/0621; C12N 5/562; C12N 2513/00

(57) ABSTRACT

The present disclosure is directed to methods of producing chimeric organoids containing cells of chimeric origin. The present disclosure is also directed to compositions comprising such chimeric organoids and the use of such chimeric organoids in high throughput screens.

### RELATED APPLICATION(S)

This application claims the benefit of and priority to U.S. Provisional Application No. 63/542,762, filed Oct. 5, 2023, U.S. Provisional Application No. 63/430,660, filed Dec. 6, 2022, and U.S. Provisional Application No. 63/431,018, filed Dec. 7, 2022. The entire teachings of the above applications are incorporated herein by reference.

### BACKGROUND OF THE INVENTION

The advent of patient-derived stem cell technology has inspired exciting avenues to study the elusive architecture and high order cognitive function of the human brain. For centuries, the tightly regulated differentiation of neuronal lineages during embryogenesis has remained unexplored. In recent decades, mouse and primate research has shed light on the developmental trajectories that lead to brain maturation, and from this framework the understanding of the complex process of brain development has advanced. However, human-specific features of the Central Nervous System (CNS) and its development cannot be completely recapitulated in animal models. For example, in the cerebral cortex, outer radial glia (oRG) progenitors are virtually absent in rodents but characteristic of the developing human cortex and potentially involved in its evolutionary expansion. Moreover, the rate of development varies significantly between species. In humans, brain development occurs in a much slower and protracted manner compared to the rapid brain development of the mouse.

The inaccessibility of fetal brain tissue has left human specific processes largely unknown and without proper avenues of exploration; thus, fundamental neurobiology research has remained lacking, and prospective cures for several neurological conditions fall short.

Over decades the field has developed ever-improving cell culture models of brain development; the recent emergence of human brain organoids represents a major advance in modeling the cellular complexity and structural features of the embryonic human brain in vitro. Brain organoids are complex 3D in vitro structures, derived from stem cell cultures that are able to replicate some of the cell type- and tissue-specific features of the brain. The accessibility of this model has led to a blossoming of neuroscience research, allowing the scientific community to gain a better understanding of the tightly controlled developmental stages that give rise to the human brain, as well as the malformation in molecular architecture that gives rise to autism spectrum disorder, schizophrenia, and many other neurological conditions.

### SUMMARY OF THE INVENTION

As described herein, a new human organoid protocol model has been developed in which cells originating from specific stages of differentiation of separate organoids are mixed or combined to generate chimeric systems (chimeric organoids or chimeroids) containing cells of different origin (e.g., from different individuals, from different species, from different regions of the brain, from tissues different than the brain, and the like). This protocol model has been applied as described herein to create several different and exemplary systems:

(1) Generation of organoids comprising cells from multiple individuals within the same organoid. This protocol and the organoids produced therefrom enable the generation of scalable systems to test the response of human cells of multiple individuals concurrently which allows for the testing of environmental, genetic and/or drug treatment effect on many cell types, from multiple people concurrently. These systems can be used to produce data relating to responses to drugs by all cells and by many people to stratify patients for clinical trials based on individual responses to defined drugs during clinical investigation.

(2) Generation of organoids that mix cells from distinct brain regions (and multiple donors) to form a “complete” and reproducible set of cells of a given brain region whereby defined, relative proportions of cell types can be controlled, and thus defined circuits can be studied-allowing investigation of higher-order physiological properties of the system with a level of precision and fidelity to the endogenous brain that is not possible with organoids and methods of the existing art.

(3) Generation of organoids in which cells of different species can be grown together to decode mechanisms that control the speed of development and develop methods to accelerate human brain development in vitro. This can also be used to identify human-specific features and properties of the brain, highlighting properties of the human brain that can (or cannot) be investigated in other species.

(4) Generation of organoids in which brain cells of one brain region are mixed with cells originating outside that region and also outside the brain (e.g., microglia, photoreceptors of the retina, cell of the vasculature, and the like). This approach enables integration in a single brain organoid of cells that would not spontaneously emerge following integration of cells that permit sensing of information from the environment, such as photoreceptors for sensing light. In some aspects, light could then be used to stimulate circuit maturation in organoids and to record circuit responses to external sensory input in the context of disease. For example, circuits generated from cells of neuropsychiatric patients (or Autism patients) could be examined to determine if they would respond differently to sensory stimulation. In some aspects, light or sound could also be used as the necessary input to generate human brain organoid-machine interfaces.

(5) Generation of organoids in which cells from different embryological origins are mixed together to recapitulate fundamental brain-body axes (e.g., endothelial, cardiac, gut cells, and the like). In some aspects, this organoid enables the investigation of more complex biology along different brain-body axes (e.g., endothelial, cardiac, gut cells, and the like). In some aspects, this organoid enables the investigation of more complex biology along different brain-body axes in the same organoid. The brain receives signals from other tissues in the body and reciprocates with signals from the brain to the other tissues (e.g., the gut-brain axes).

All of the above described organoids can be adapted to incorporate cells from multiple cell types and/or multiple donors.

This protocol and the organoids produced therefrom enable scalable systems to test the response of human cells of different individuals concurrently, allowing testing of environmental, genetic and drug treatment effect on many cell types, from multiple people concurrently. These systems can be used to produce data relating to responses to drugs by all cells and by many people to build predictive models of drug response, for example. In addition, the protocol, organoids and systems described herein enable the production of organoids in which the proportions of each cell type can be controlled, and thus defined circuits can be studied—not possible with organoids and methods of the art.

Described herein are protocols that advance current patterned organoid systems to fill gaps in the field. First, an innovative protocol is described that can be used to derive brain organoids (e.g., cortical organoids) containing the genetic background of multiple individuals, i.e., organoids started with stem cells of multiple individuals and that end up containing cell types of the brain from many individuals, co-developing and growing together. It is not currently possible to generate 3D organoids from multiple donors by mixing pluripotent stem cells because in each of these systems some stem cell lines take over the organoids, eliminating multi-donor final representation. Described herein is a method developed to solve this problem by producing multi-donor organoids that are produced by mixing neural stem and progenitors cells (NSCs, NPCs) at distinct stages of development (e.g., starting at 15 DIV and ending at 28 DIV). By fine-tuning the time of mixing of NSCs and NPCs from multiple donor lines and based on what brain region the NSCs and NPCs come from, a highly reproducible method to obtain multi-donor chimeric organoids was developed. This model scales up dramatically the number of cell types and individual donors that can be investigated within one organoid, increasing throughput and reducing batch variation. Chimeroids will be pivotal in understanding the varied response to pharmaceuticals, genetic insults or neurotoxic compounds on a population-wide level, at scale, without the need to grow organoids for each patient and reducing the culture variability for each individual.

Second, a telencephalic (rostral part of the brain) chimeric organoid has been produced in an exemplary embodiment. This particular organoid model combines cells that come from the cortex (pallium) and the basal ganglia (subpallium), which are normally co-present within the endogenous cerebral cortex (though the invention is not limited to this particular combination of cell types, for example, cells from other brain regions can also be mixed together). By mixing such pallial and subpallial lineages the correct proportion of inhibitory and excitatory neurons found in vivo is recapitulated within the same final organoid. In this specific example, different proportions of starting NSCs numbers from the pallium and the subpallium were tested to obtain a chimeroid (e.g., after 3-6 months in culture) that contains a ratio of subpallial inhibitory interneurons to pallial excitatory neurons (e.g., at about a 1:5 ratio) (as observed in the endogenous cerebral cortex). NSCs from different brain regions may divide at different rates, and so the exact numbers of NSCs to mix should be established empirically at the start of the protocol. This model enables investigation of the local cortical microcircuit, modeling development, function and multiple neuropsychiatric disorders in which the delicate balance of excitation and inhibition of the brain is likely aberrant.

Additionally described herein in an exemplary embodiment is a chimeric organoid model that combines neural stem cells from different species (e.g., from mouse and human) providing a fundamental model for understanding the intrinsic and extrinsic molecular cues that regulate the slow pace of development and large expansion of the human brain. The model will enable investigation of the mechanisms controlling the speed of human cortical development to inform new protocols to accelerate the formation of human cortical organoids in vitro.

Additionally described herein in an exemplary embodiment is a chimeric organoid model in which brain cells of one or more brain regions are mixed with cells originating outside the brain (e.g., microglia, photoreceptors of the retina, cells of the vasculature, and the like). This approach may enable integration in a single brain organoid of cells of the brain immune system (e.g., microglia) to study synaptic pruning and circuit refinement. In some aspects, it would permit sensing of information from the environment, e.g., through photoreceptors sensing light. Light may be used to stimulate circuit maturation in organoids and to record circuit responses to external sensory input in the context of disease. In some aspects, circuits generated from cells of neuropsychiatric patients (or Autism patients) are assessed to determine if the circuits would respond differently to sensory stimulation. In some aspects, light or sound could act as the necessary input to generate human brain organoid-machine interfaces. The invention also provides methods for generating a “chimeroid-machine interface.” More particularly, a chimeroid can be interfaced with a computer, and spontaneous and induced sensory circuit activity input can be analyzed. The software can output instructions to the stimulus-generating device in a loop, thereby mimicking sensory-response learning. See et al. Kagan B J, et al. “In vitro neurons learn and exhibit sentience when embodied in a simulated game-world.” Neuron. 2022 Dec. 7; 110 (23): 3952-3969.e8; Smirnova L, et al. “Organoid intelligence (OI): the new frontier in biocomputing and intelligence-in-a-dish.” Frontiers in Science. 2023; doi: 10.3389/fsci.2023.1017235, incorporated herein by reference in its entirety.

The invention also provides methods for screening of molecular, cellular and network-level defects associated with diseases that affect many cell types, brain regions and organs, including complex mental diseases. Chimeroids generated from patient cells can be screened for defects in cellular composition, as well as for differences in spontaneous and induced network activity. Brain chimeroids can also be screened for response to stimuli and performance during chimeroid-machine interface learning sessions (“capacity to learn”). Such screening methods can also assess the impact of agents on brain function, particularly at the molecular, cellular and/or network level.

In some embodiments, the chimeric brain organoid further includes cell types including, but not limited to, one or more of microglia, oligodendrocytes, endothelial cells, cells of the immune system and stromal cells. In some embodiments, the compositions further include differentiated human cell types selected from the group consisting of cortical neurons, subcortical neurons, and sensory cells. In some embodiments, the sensory cells are one or more cells bearing one or more sensory receptors selected from the group consisting of photoreceptors, auditory receptors, olfactory receptors, tactile receptors, and taste receptors. In some embodiments, the one or more sensory receptors are capable of responding to a detected stimulus. In some embodiments, the chimeroid compositions comprise mature retinal tissue, e.g., the chimeroid compositions may comprise one or more of each cell type of the human retina. In some embodiments, cells that are genetically modified to carry disease risk genes/disease genetics can be mixed with control cells to decode the target cell types affected and intrinsic vs extrinsic mechanisms of disease. In some embodiments, cell lines that have strong differentiation or proliferation biases that preclude their use in organoids can be mixed with cells that do not carry such biases and may be “compensated” so that they can now be used to grow an organoid.

In some embodiments, the chimeric brain organoid further includes a neural circuit. In some embodiments, the sensory cells form a neural network with one or more additional cells within the organoid. In some embodiments, the neural network includes a functional connection between the neural circuit and the sensory cell. In some embodiments, the neural network is capable of exhibiting a response to external physiological stimuli. For example, the physiological stimuli may be one or more stimuli selected from the group consisting of light, sound, taste, smell, temperature and touch.

In some embodiments, the present invention is directed to in vitro methods of producing a chimeric brain organoid, the methods comprising forming embryoid bodies from cells (embryonic stem cells, induced pluripotent cells, patient-derived induced pluripotent stem cells, etc.), producing a patterned organoid (e.g., a dorsally patterned organoid) from each of said embryoid bodies, dissociating at least a portion of each of said patterned organoids to produce dissociated single cells, combining dissociated single cells from each of the patterned organoids, and culturing the combined dissociated single cells under conditions and for a time sufficient to produce a three-dimensional chimeric brain organoid. In some embodiments, the cells are human cells. In some embodiments, the patient-derived induced pluripotent stem cells are derived from a patient exhibiting a complex disease affecting brain activity. In some exemplary embodiments, the complex disease is selected from the group consisting of epilepsy, schizophrenia, bipolar disorder and autism spectrum disorder (ASD).

In some embodiments, the present inventions are directed to generating three dimensional chimeric brain organoids by the methods disclosed herein and isolating one or more cell types from the organoids. The isolated one or more cell types may be further cultured and optionally used for screening or disease modeling.

In some embodiments, the present inventions are directed to methods for screening for disease, the methods comprising generating a three dimensional chimeric brain organoid from patient-derived induced pluripotent stem cells, and screening for dysregulation of cell types, their development, their interactions, and of spontaneous activity or defects of stimulus-induced activity in circuits. In some embodiments, the cerebral organoids are co-cultured according to the methods described herein with other cell types including, but not limited to, one or more of microglia, oligodendrocytes, endothelial cells, cells of the immune system and stromal cells. In some embodiments, the disease is a neurological, neurodevelopmental, neuropsychological, neuropsychiatric, neurodegenerative, or neuropsychopharmacological disease. In some embodiments, the disease is a neuropsychiatric disease.

In still other embodiments, disclosed herein are chimeric brain organoid-machine interfaces comprising a multi-probe electrode array configured to collect electrophysiological signals from neural tissue, a first processor connected to the multi-probe electrode array, a second processor coupled to a stimulus-generating device, and machine executable instructions configured to decode circuit response and instruct feedback stimulation to the sensory generating device.

In some embodiments, the first processor is configured to collect and store the electrophysiological signals. In some embodiments, the stimulus-generating device is a light-emitting diode (LED). In some embodiments, the stimulus-generating device generates a stimulus selected from the group consisting of a visual stimulus, an auditory stimulus, an olfactory stimulus, a taste stimulus, a temperature and a touch stimulus. In some embodiments, the chimeroid-machine interface measures the neural tissue for spontaneous activity in response to the stimulus. In certain embodiments, the chimeroid-machine interface measures the neural tissue for network activity in response to the stimulus. In some embodiments, the chimeroid-machine interface screens the neural tissue for dysregulation of spontaneous activity in response to the stimulus. In some embodiments, the chimeroid-machine interface screens the neural tissue for defects of synaptic and network activity in response to the stimulus. In some embodiments, the chimeroid-machine interface screens the neural tissue for defects in performance of the neural tissue during a learning session.

In some embodiments, the multi-donor chimeroids can be used to stratify patients ahead of clinical trials and/or investigation. In some aspects, chimeroids may be used to read at scale the responses by cells of different individuals to therapeutics of interest. Data can then be used directly, across all patients that are part of a trial to assess the likelihood by which patient(s) would respond to the drug(s). As a result of the scalable nature of the multi-donor chimeroids described herein, sample responses can be obtained for many cell types and for many individuals, and big-data can be generated on cell responses that can fuel AI-driven generative, predictive models of drug (and perturbation) response by cell type and individual.

### BRIEF DESCRIPTION OF THE DRAWINGS

The patent or application file contains at least one drawing executed in color. Copies of this patent or patent application publication with color drawings will be provided by the Office upon request and payment of the necessary fee.

FIGS. 1A-1M demonstrate NSC-Chimeroids maintain donor representation across cell types. FIG. 1A provides a schematic of the PSC-Chimeroids protocol. PSCs: pluripotent stem cells, DIV: days in vitro. FIG. 1B shows immunolabelling of MAP2, EMX1 and NESTIN of cortical PSC-Chimeroids at 1 month (mo), whole organoid and zoom-in images. Scale bar: 500 μm and 250 μm respectively. FIG. 1C provides brightfield images of cortical PSC-Chimeroids at 3 mo. Scale bar: 1 mm. FIG. 1D shows 3-mo PSC-Chimeroids analyzed by single-cell RNA-seq and demultiplexed using demuxlet show unequal distribution of donor lines; donor is indicated by colors. B=batch and R=biological replicate. FIG. 1E provides a schematic of the NSC-Chimeroids protocol. NSCs: Neural Stem Cells. FIG. 1F shows NSC-Chimeroids demultiplexed by donor after single-cell RNA-seq at 3 mo. FIG. 1G, Left panel provides a brightfield image of cortical NSC-Chimeroids at 1 mo. Scale bar: 1 mm. FIG. 1G, Right panels shows immunolabelling of 1-mo cortical NSC-Chimeroids showing cortical progenitors (FOXG1), corticofugal pyramidal neurons (CTIP2), and tight junctions formed by neural progenitors (ZO-1). Scale bar: 100 μm. FIG. 1H, Left panel provides a brightfield image of 3-mo cortical NSC-Chimeroids (n=3; left panel). Scale bar: 1 mm. FIG. 1H, Right panels show immunolabelling of 3-mo cortical NSC-Chimeroids showing cortical progenitors (EMX1) and the neuronal dendritic marker MAP2. Scale bar: 500 μm. FIG. 1I shows UMAP of integrated NSC-Chimeroids, color-coded by annotated cell type (upper panel) and donor line as determined with demuxlet (lower panel). FIGS. 1J-1K show UMAPs split by donor for two different mixes (Mix 1, 4 donors (FIG. 1J) and Mix 2, 5 donors (FIG. 1K)). FIG. 1L provides bar plots of cell-type proportions in NSC-Chimeroids, demultiplexed by donor. Whiskers show upper and lower quartile values across replicates (n=7 for CW, H1, Mito210, and PGP1; n=3 for 11a). FIG. 1M provides stacked bar plots of cell-type proportion for each donor within Mix 1 and Mix 2.

FIGS. 2A-2K demonstrate NSC- and NPC-Chimeroids control hyperproliferative states of pluripotent stem cells. FIG. 2A shows 3-mo NSC-Chimeroids analyzed by single-cell RNA-seq and demultiplexed by donor: Mix 3 and Mix 4 include the highly proliferative donor GM. FIG. 2B shows 3-mo NSC-Chimeroid UMAPs color-coded by annotated cell type for Mix 3 (4 donors) and Mix 4 (5 donors). FIG. 2C provides a bar plot with cell type and donor composition; the width of each bar corresponds to the donor proportion. FIG. 2D provides brightfield images of 3-mo cortical NSC-Chimeroids. Scale bar: 1 mm. FIG. 2E shows UMAPs split by donor for Mix 3 (4 donors). FIG. 2F provides a density plot of pseudotime by cell line, calculated using Monocle3 with all Cycling cells as roots. FIG. 2G provides a schematic of protocol for NPC-Chimeroids. DIF=Differentiation. FIG. 2H, Upper-left panel provides a brightfield image of 3-mo NSC-Chimeroids. Scale bar: 1 mm. FIG. 2H also provides immunohistochemistry of 3-mo NPC-Chimeroids showing SOX2 (progenitors), CTIP2 (cortical neurons) and MAP2 (dendrites). Scale bar: 500 μm. FIGS. 21-2J show UMAP representation of NPC-Chimeroids integrated (FIG. 2I) and split by donor (FIG. 2J). FIG. 2K provides barplots of cell type and donor composition which demonstrate balanced growth and proper cortical development; the width of each bar corresponds to the donor proportion.

FIGS. 3A-3F demonstrate chimeroids are reproducible and display proper cell-type composition. FIG. 3A provides a schematic of cell types in the developing cortex. FIG. 3B shows dot plots showing expression of marker genes across common cell types in a previously published atlas of cortical organoid development (‘atlas’) (Uzquiano et al., Cell, 2022), NSC-Chimeroids, single-donor (SD) NSC-Chimeroids, and NPC-Chimeroids. Point size indicates the percentage of cells which express each marker gene, and shade represents average expression values. Color indicates annotated cell type. FIG. 3C shows Pearson correlation coefficients for normalized marker gene expression between human fetal cortical cell types and corresponding cell types in atlas organoids, NSC-Chimeroids, single-donor NSC-Chimeroids, and NPC-Chimeroids. FIG. 3D shows Rank-Rank Hypergeometric Overlap (RR-HO) plots comparing the expression signatures of CFuPN and oRG (upper and lower panels respectively) in multi-donor Chimeroids to their counterparts in atlas cortical organoids or endogenous fetal tissue. The horizontal axes represent genes significantly differentially expressed in a given Chimeroid cell type compared to all other Chimeroid cells, ranked from most upregulated to most downregulated; the vertical axes represent similarly ranked lists of differentially-expressed genes for atlas organoid or fetal cells. Color at a given position represents the significance (negative log p-value) of the overlap of the gene lists up to that point, as calculated by Fisher's exact tests. High significance (i.e., red color) in the lower left and upper right quadrants indicates strong concordance between the expression profiles which define the corresponding cell types. FIG. 3E, Left shows cell type annotations assigned to Chimeroid cells using two different reference datasets: an atlas of human cortical organoid cells (top left), and endogenous human fetal cortex cells (lower left). FIG. 3E, Right shows the converse: cell type annotations using Chimeroid cells as a reference, mapped onto cells from the cortical organoid atlas (top right) and from fetal tissue (lower right). The vertical axis groups cells by manually-annotated cell type (based on marker gene expression and clustering in the query dataset). The horizontal axis groups cells by the transferred label from the reference dataset. Point size and color indicates the percentage of cells which were assigned a given label. FIG. 3F provides Adjusted Mutual Information (AMI) values within batches between cell types and individual cortical organoids from the atlas, individual single-donor NSC-Chimeroids, and individual donors within each Mix 1, 2, 4 NSC-Chimeroid and Mix 3 NPC-Chimeroids. Lower values indicate lower variability in cell type composition between replicates. Dotted lines represent AMI values between replicates within three datasets of endogenous human fetal cortex (Polioudakis et al., Neuron, 2019; Trevino et al., Cell, 2021; Uzquiano et al., Cell, 2022).

FIGS. 4A-4I demonstrate NSC-Chimeroids model treatment-specific alterations. FIG. 4A provides a schematic of the EtOH and VPA experimental design. FIGS. 4B-4C show integrated UMAPs colored by annotated cell type for control (FIG. 4B) and treated (FIG. 4C) conditions, with corresponding brightfield images of 3-mo NSC-Chimeroids. Scale bar: 1 mm. FIGS. 4D-4E show UMAPs of EtOH (FIG. 4D) and VPA (FIG. 4E) treated NSC-Chimeroids, split by mixes and replicates. FIG. 4F provides bar plots of cell-type proportions in control multi-donor NSC-Chimeroids in control, (grey) and treated with EtOH (purple) or VPA (yellow). Whiskers show upper and lower quartiles. Plot is split into three facets to allow rescaling of the y-axis (indicating percent abundance). Significance of differences between treated and control Chimeroids was calculated using a negative binomial generalized linear model. FDR-adjusted p-values: *−<0.1; **−<0.01; ***−<0.001. FIG. 4G shows changes in cell-type proportions in single-donor Chimeroids from the corresponding lines, as in FIG. 4F. FIG. 4H, Left provides UMAP showing the localization of cells from control multi-donor Chimeroids (grey) and cells from multi-donor Chimeroids treated with VPA (yellow) and EtOH (violet). FIG. 4H, Right provides UMAP showing overlapping neighborhoods of cells, as calculated using Milo. Red and blue colors indicate neighborhoods with significant enrichment for EtOH and VPA-treated cells or control cells, respectively. Point size indicates the number of cells in a neighborhood, and edge thickness indicates the number of cells shared between pairs of neighborhoods. FIG. 4I provides beeswarm plot showing shifts in the composition of neighborhoods of cells in response to EtOH and VPA treatment, grouped by the cell-type identity of those neighborhoods. Each point represents a neighborhood of 50-200 cells with similar gene expression profiles. The vertical axis indicates the enrichment of EtOH and VPA-treated cells within a neighborhood, with positive log 2 fold change values indicating more than expected EtOH or VPA-treated cells, and negative values indicating fewer than expected EtOH or VPA-treated cells. Neighborhoods are colored based on statistical significance of that enrichment: grey, not significantly different from random; red, significant over-enrichment; blue, significant under-enrichment. If most neighborhoods within a cell type collectively shift up or down, it implies an overall gain or loss, respectively, of that cell type in EtOH or VPA-treated Chimeroids. Cell types with neighborhoods that form long tails of both overenrichment and underenrichment are likely to have EtOH or VPA-induced changes in expression profile, without necessarily changing in abundance.

FIGS. 5A-5D demonstrate NSC-Chimeroids display differential donor-specific susceptibility. FIG. 5A shows UMAPs of control, EtOH-, and VPA-treated Chimeroids split by donor and colored by cell type. FIG. 5B provides a bar plot of median normalized donor proportions in control NSC-Chimeroids (grey) and treated with EtOH (purple) or VPA (yellow). The y-axis represents the ratio of the measured donor abundance in each Chimeroid to the expected abundance if all donors within a mix were equally represented (i.e., 25% in Mix 1 or 20% in Mix 2). Whiskers show upper and lower quartiles. Significance of differences between treated and control Chimeroids was calculated by applying a negative binomial generalized mixed effects model to the cell counts per donor. FDR-adjusted p-values: *−<0.1; **−<0.01; *-<0.001. FIG. 5C provides a bar plot of VPA-induced changes in cell type proportions, split by donor. Whiskers show upper and lower quartiles. Significance was calculated using a negative binomial generalized mixed effects model, with treatment as a fixed effect, Chimeroid-of-origin as a random effect, donor as a random effect, and treatment-by-donor as a random interaction. FDR-adjusted p-values for the random interaction were calculated via likelihood ratio test against a model without the treatment-by-donor random interaction. For cell types with significant random interactions (ORG, IP, and CPN), post-hoc pairwise comparisons were performed between donors using the same models; significant pairwise differences in response to treatment are identified with brackets: *−<0.1; **−<0.01; ***−<0.001. FIG. 5D shows Principal Component Analysis (PCA) performed on the gene expression matrix of different clusters from VPA-treated (triangles) and untreated (circles) NSC-Chimeroids, pseudobulked by donor (indicated by color) and replicate, both using all cells (top left) and subsetted by cell type. Data points separate in PC1 and PC2 by donor and treatment, except in the case of IP and aRG, where they separate in PC2 and PC3. Corresponding right panels: violin plots showing the pairwise Euclidean distance in PCA space between treated and untreated pseudobulked data points from each donor. Larger distances imply that VPA treatment induces larger changes in expression profiles. Significance calculated by Analysis of Variance (ANOVA), followed by Tukey post-hoc pairwise tests.

FIGS. 6A-6F demonstrate PSC Chimeroids display uneven donor composition but proper cell type composition. FIG. 6A provides a schematic of the PSC-Chimeroid protocol. FIG. 6B provides stacked bar plots showing donor composition, as measured via Census-seq from low-pass whole-genome sequencing, in PSC-Chimeroids from various mixes and timepoints, labeled as the number of days since the day of the seeding (DIVO). For the DIVO and DIV1 time points, n=4 Chimeroids have been pooled together. FIG. 6C, Left panel provides a brightfield image of 3-mo PSC-Chimeroids; scale bar: 1 mm. FIG. 6C, Right panel provides immunolabelling of 3-mo PSC-Chimeroid with SATB2, TBR1 and MAP2. Scale bar: 500 μm. FIG. 6D shows UMAP of integrated PSC-Chimeroids at 3 mo, colored by annotated cell type (left panel) and donor line as determined with demuxlet (right panel). FIG. 6E shows UMAPs split by donor for two different replicates. FIG. 6F provides barplots of cell type proportions in PSC Chimeroids (n=2), demultiplexed by donor. DIV, days in vitro.

FIGS. 7A-7C demonstrate NSC-Chimeroids created by aggregating different numbers of cells display variable development. FIG. 7A provides brightfield images of single-donor H1 NSC-Chimeroids seeded with 9,000 (upper) and 12,000 (lower) cells/well at day 3 and day 16 after reaggregation (left panels), and brightfield images of single donor PGP1 NSC-Chimeroids seeded with 9,000 (top left), 12,000 (bottom left), and 20,000 (top right) cells/well at day 3 and day 16 after reaggregation (right panels). Scale bar: 500 μm. Barplot shows growth over time of PGP1 NSC-Chimeroids compared across all different cell counts at aggregation (bottom right). Error bars show distribution of values. Significance was calculated using ANOVA followed by Tukey post-hoc pairwise tests. P-values: ***<−0.001; ****−<0.0001. FIG. 7B shows immunolabelling of PGP1 single-donor NSC-Chimeroids made by aggregating the indicated number of cells, at DIV35. Upper images display whole organoids; lower images, enlargement of indicated portions. Lefthand panel, immunolabelling for SOX2 (progenitors), MAP2 (neuronal dendrites), and TBR1 (deep layer neurons). Righthand panel, immunolabelling for SOX2, ZO-1 (tight gap junctions), and EMX1 (dorsal cortical progenitors). Scale bar: 500 μm (upper) and 125 μm (lower). Arrowheads indicate non-cortical (grey) and cortical (white) regions. FIG. 7C, Top panel provides whole-organoid brightfield images of H1 single-donor NPC-Chimeroids seeded with 100,000 cells at aggregation, at 18 and 35 days after mixing. Scale bar: 500 μm. FIG. 7C, Bottom panel shows immunolabelling of H1 single-donor NPC-Chimeroids showing SOX2, ZO-1, and EMX1. Scale bar: 500 μm and 125 μm (zoom-in).

FIGS. 8A-8E demonstrate NSC-Chimeroids develop proper cell-type composition across multiple donors. FIG. 8A provides stacked barplots showing the donor composition, as measured via Census-seq from low-pass whole-genome sequencing, of multi-donor NSC-Chimeroids from various mixes and at various timepoints, labeled as the number of days since reaggregation (DIV; for the DIVO and DIV1 time points, n=4 Chimeroids have been pooled together). FIGS. 8B-8E show 3-mo NSC-Chimeroids UMAPs split by donor (FIGS. 8B-8C) and stacked barplots of donor contributions for each cell type (FIGS. 8D-8E) for Mix 1 (4 donors; FIG. 8B and FIG. 8D) and Mix 2 (5 donors; FIG. 8C and FIG. 8E).

FIGS. 9A-9B demonstrate individual donors within NSC-Chimeroids maintain consistent proportions of cortical cell types. FIGS. 9A-9B show distribution of cell types across individual donors, across two independent mixes, Mix 1 (FIG. 9A) and Mix 2 at 3 mo (FIG. 9B). Donors were demultiplexed using demuxlet.

FIGS. 10A-10C demonstrate Chimeroids display similar neural progenitor complexity as single-donor organoids and fetal tissue. Rank-Rank Hypergeometric Overlap (RR-HO) plots comparing the expression signatures of progenitor types in multi-donor Chimeroids to all cell types in single-donor chimeroids, atlas cortical organoids or endogenous human fetal tissue. The horizontal axes represent lists of marker genes for multi-donor Chimeroid progenitor types (FIG. 10A-aRG; FIG. 10B-ORG; FIG. 10C-IP) compared to all other Chimeroid cells, ranked from most upregulated to most downregulated; the vertical axes represent similarly ranked lists of marker genes for, single-donor Chimeroids, atlas organoids or human fetal cells. Color at a given position represents the significance (negative log p-value) of the overlap of the gene lists up to that point, as calculated by Fisher's exact tests. High significance (i.e., red color) in the lower left and upper right quadrants indicates strong concordance between the expression profiles which define the compared cell types.

FIGS. 11A-11B demonstrate Chimeroids display similar neuronal complexity as single-donor organoids and fetal tissue. RR-HO plots comparing the expression signatures of pyramidal neuron types in multi-donor Chimeroids to all cell types in single-donor Chimeroids, atlas cortical organoids or endogenous human fetal tissue. The horizontal axes represent lists of marker genes for multi-donor Chimeroid pyramidal neuron types (FIG. 11A—CfuPN; FIG. 11B—CPN) compared to all other Chimeroid cells, ranked from most upregulated to most downregulated; the vertical axes represent similarly ranked lists of marker genes for, single-donor Chimeroids, atlas organoids or human fetal cells.

FIGS. 12A-12B demonstrate VPA and EtOH treatment differently affect cell-type composition in NSC-Chimeroids. FIG. 12A provides stacked barplots showing cell type and donor composition for treated and control Chimeroids; the width of each bar corresponds to the proportion of the indicated donor in Mix 1 (4 donors) and Mix 2 (5 donors). FIG. 12B provides brightfield images of whole cortical NSC-Chimeroids at 3 mo, in the EtOH and VPA treatment conditions. Scale bar: 1 mm.

FIGS. 13A-13B demonstrate VPA-treated Chimeroids develop an immature interneuron population. Immunolabelling of multi donor NSC-Chimeroids (Mix 1, FIG. 13A and Mix 2, FIG. 13B). Top panels, MAP2 (neuronal dendrites), EMX1 (cortical progenitors), and DLX2 (GABAergic cells). Bottom panels, DCX (migrating neurons), SATB2 (upper layers cortical neurons), and CTIP2 (deep layers cortical neurons). Scale bar: 100 μm.

FIGS. 14A-14B demonstrate VPA and EtOH treatment cause alterations in the transcriptome profile of single-donor Chimeroids. FIG. 14A provides stacked barplots showing cell type composition for control and treated single-donor Chimeroids. FIG. 14B, Upper-left panel shows cell-type specific changes in single-donor Chimeroids treated with VPA (yellow) vs control (grey). FIG. 14B, Upper-right panel shows UMAP showing overlapping neighborhoods of cells, as calculated using Milo. Red and blue colors indicate neighborhoods with significant enrichment for VPA-treated cells or control cells, respectively. Point size indicates the number of cells in a neighborhood, and edge thickness indicates the number of cells shared between pairs of neighborhoods. FIG. 14B, Bottom panel provides a beeswarm plot showing shifts in the composition of neighborhoods of cells in response to VPA treatment in single-donor Chimeroids, grouped by the cell-type identity of those neighborhoods. Each point represents a neighborhood of 50-200 cells with similar gene expression profiles. The vertical axis indicates the enrichment of VPA-treated cells within a neighborhood, with positive log-fold change values indicating more than expected VPA-treated cells, and negative values indicating fewer than expected VPA-treated cells. Neighborhoods are colored based on statistical significance of that enrichment: grey, not significantly different from random; red, significant over-enrichment; blue, significant under-enrichment. If most neighborhoods within a cell type collectively shift up or down, it implies an overall gain or loss, respectively, of that cell type in VPA-treated Chimeroids. Cell types with neighborhoods that form long tails of both over-enrichment and under-enrichment are likely to have treatment-induced changes in expression profile, without necessarily changing in abundance.

FIG. 15 provides brightfield images of NSC-chimeroids generated utilizing the protocol provided in Example 3. Images of the NSC-chimeroids are provided at 1 day, 3 days, and 35 days after mixing.

FIG. 16 provides brightfield images (top panel) of Thw-NSC-chimeroids (DIV 16, DIV 35, DIV 43, and DIV 60) and Thw-NPC-chimeroids (DIV 30, DIV 35, and DIV 60) generated utilizing the protocol provided in Example 4. In addition, FIG. 16 shows immunolabeling of Thw-NSC-chimeroids at DIV 60 with MAP2 (neuronal dendrites), TBR1 (deep layer neurons), and SATB2 (upper layers cortical neurons).

FIG. 17 demonstrates human cortical organoids as models of neurodevelopment and disease. A schematic is provided for obtaining human cortical organoids. The human cortical organoids provide reliable and reproducible models for research.

FIG. 18 shows reproducible cell diversity in human brain organoids.

FIG. 19 demonstrates reliable corticogenesis in human brain organoids.

FIG. 20 shows modeling ASD risk gene mutations in brain organoids.

FIG. 21 shows that genetic background modulates expressivity of mutant phenotypes.

FIG. 22 demonstrates an at scale at-scale investigation of inter-individual variability, such as diverse human genetic backgrounds and multiple risk genes.

FIG. 23 shows PSC-chimeroids form multi-donor human cortical organoids.

FIG. 24 shows PSC-chimeroids exhibit unbalanced donor contribution. However, NSC-chimeroids are shown to maintain donor representation across cell types.

FIG. 25 shows NSC-chimeroids differentiate into proper brain cortical cell types.

FIG. 26 provides a protocol for obtain NSC-chimeroids. Aggregation after patterning ameliorates growth differential.

FIG. 27 shows NSC-chimeroids maintain donor representation across cell types.

FIG. 28 shows NSC chimeroids maintain donor representation across cell types following the proper developmental trajectories. The chimeroids provide reliable and reproducible models.

FIG. 29 demonstrates NSC-chimeroids rescue fate divergency.

FIG. 30 shows NSC-chimeroids maintain donor contribution.

FIG. 31 shows PSC lines from different donors differ in proliferation.

FIG. 32 demonstrates that combining lines at the NPC stage overcomes overgrowth.

FIG. 33 shows NPC-chimeroids can be agnostic of the stem cell line. NSC- and NPC-chimeroids may control hyperproliferative states of pluripotent stem cells.

FIG. 34 shows NSC and NPC chimeroids follow the proper developmental trajectories.

FIG. 35 demonstrates that neurotoxic triggers, such as EtOH and VPA, can be used with human brain cortical chimeroids to dissect inter-individual variability in susceptibility to neurotoxic triggers. There is shown to be inter-individual variability in neurotoxin susceptibility.

FIG. 36 shows chimeroids are treated with neurotoxic triggers to assess inter-individual variability in susceptibility to such triggers.

FIG. 37 shows EtOH and VA differentially affect specific cell types.

FIG. 38 shows EtOH and VA differentially affect specific molecular pathways.

FIG. 39 shows EtOH and VA differentially affect donor susceptibility and different proportions of cells per donor.

FIG. 40 shows EtOH and VA differentially affect specific molecular pathways.

FIG. 41 shows VPA differentially affects specific donors.

FIG. 42 provides a summary of research that has been performed on chimeroids.

FIG. 43 provides a summary of objectives for obtaining various chimeroids.

FIGS. 44A-44D show multidonor chimeroids are reproducible. FIG. 44A provides a protocol schematic for producing multidonor chimeroids. FIG. 44B shows reproducibility and donor contribution for 1-month chimeroids. At 1-month, multidonor chimeroids display even donor composition and are reproducible. Cell type composition shows proper early cortical signature. FIG. 44C shows reproducibility and donor contribution of 3-month chimeroids. 3-month iPSC chimeroids are composed by only one donor. Cell type composition shows aberrant cortical signature. FIG. 44D shows reproducibility and donor contributions of 3-month chimeroids. 3-month NSC-chimeroids are composed by all donors and are reproducible. Cell type composition shows proper cortical signature.

FIGS. 45A-45D show telencephalic chimeroids are reproducible. FIG. 45A provides a protocol for producing telencephalic chimeroids. FIG. 45B shows developmental trajectory of telencephalic chimeroids. FIG. 45C shows subpallial structures and pallial cell-types and organization. FIG. 45D shows single cell RNAseq of telencephalic chimeroids. 2-month telencephalic chimeroids display proper molecular signatures of pallial and subpallial cell populations.

FIGS. 46A-46D show human-mouse chimeras. FIG. 46A provides a protocol for producing human-mouse chimeroids. FIGS. 46B-46D show the results of human-mouse chimeroids at various stages of development.

### DETAILED DESCRIPTION OF THE INVENTION

Inter-individual genetic variation affects the susceptibility to and progression of many diseases. Efforts to study the molecular mechanisms mediating the impact of human genetic variation on normal development and disease phenotypes are limited, however, by the paucity of faithful cellular human models, and the difficulty of scaling current systems to represent multiple individuals. Described herein are human brain “chimeroids,” a highly reproducible, multi-donor human brain cortical organoid model. In some aspects, the chimeroids were leveraged to investigate inter-individual variation in susceptibility to neurotoxic stressors that exhibit high clinical phenotypic variability (e.g., ethanol and the anti-epileptic drug valproic acid). The chimeroids described herein are shown to provide a scalable system for high-throughput investigation of the contribution of human genetic variation to brain development and disease.

### Chimeric Organoids

### Multidonor Chimeroids

It has previously not been possible to grow stem cell-derived models (e.g., 3D stem cell-derived models) of the brain (and other organs) by mixing stem cells of multiple individuals. Yet, this is necessary to enable investigation of inter-human differences in responses to diseases and drugs. It is also necessary to enable studies that measure the response of human cells from specific patients to environmental, genetic or drug treatment, for example, impeding the development of models of human cell responses that can have predictive value. Described herein are methods for generating human cortical organoids that contain cells derived from multiple donors. In order for this model to be useful as an experimental system, cells from each person must undergo development in a reproducible manner without major divergence in the environment of other donor lines and without having lines that “take over” the organoid.

It is well known that stem cells have varying properties of growth, differentiation, and maturation; these properties manifest in the propensity of particular lines to take over the entire system in culture. This has hampered the development of “villages” in organoids. Reproducible multi-donor organoids have been created by methods described herein comprising first patterning each donor line separately before mixing them and beginning differentiation. By fine tuning the timing and proportions of mixing protocols, organoids containing all donor cells of origin were generated and able to grow by keeping a representation of each line. This protocol has the potential to remarkably reduce batch-to-batch variability inherent in cell line differentiation and create a scalable tool to screen drugs/neurotoxicity on a variety of donor genetic backgrounds.

In some aspects, multi-donor organoids are produced by mixing neural stem and progenitor cells (NSCs, NPCs) at distinct stages of development. For example, the NSCs and NPCs may be mixed starting at 15 DIV and ending at 28 DIV. By fine tuning the time of mixing of NSCs and NPCs from multiple donor lines, and based on what brain region the NSCs and NPCs come from, a highly reproducible method to obtain multi-donor chimeric organoids was developed. In one aspect, a range of 50-85% of ventral cells (e.g., derived from organoids differentiated using the protocol described in Sartore et al., incorporated herein by reference in its entirety, at day 24-27) is mixed with 15-50% of dorsal cells (derived from organoids differentiated using the protocol described in Velasco, et al., incorporated herein by reference in its entirety, at day 23-27). In some aspects, cells from different donors are provided at a 1:1:1 ratio. In other aspects, cells from different donors are provided at a variable ratio, where the ratio is determined by the calculating the proliferative capacity of the NSC of each donor by calculating cell-cycle scores per donor line. An exemplary protocol is outlined in Example 2.

### Telencephalic Chimeroids

Further extending the organoid model, described herein is a protocol in which combined Pallial and Subpallial organoids were generated. The cerebral cortex is largely composed of two neuronal populations: GABA-producing (inhibitory) interneurons, primarily generated in the ventral telencephalon, and pyramidal neurons that produce glutamate (excitatory), primarily located in the dorsal telencephalon. During development, interneurons perform long range migration to their target dorsal (in the cortex) region in a controlled manner in order to build the cortical microcircuit. To model this in utero phenomena, a reproducible and stable telencephalic human chimeroid protocol was developed as described herein in which cells of pallial and subpallial identity are mixed to achieve predictable final proportions.

Compared to current fusion models (assembloids), in which organoids (rather than cells) of different brain regions are fused, the protocol described herein enables precise control of the final proportion of cell types (neuron types) in the organoid. This enables predictable outcomes and the investigation of circuits among the correct proportions of each neuron type, something that has not previously been possible.

In some aspects, different proportions or ratios of starting NSC numbers from the pallium and the subpallium may be examined. For example, different proportions of starting NSCs from the pallium and the subpallium may be examined so as to obtain a chimeroid that contains a 1:5 ratio of subpallial inhibitor interneurons to pallial excitatory neurons (as observed in the endogenous cerebral cortex) after a period of 3-6 months in culture. In some aspects, the NSCs from different brain regions divide at different rates, and thus the exact numbers of NSCs to mix may be established empirically at the start of the protocol. In some aspects, a pallial: subpallial mix has a final ratio of about 1:5. In one aspect, a range of 50-85% of ventral cells (e.g., derived from organoids differentiated using the protocol described in Sartore et al., incorporated herein by reference in its entirety, at day 24-27) is mixed with 15-50% of dorsal cells (derived from organoids differentiated using the protocol described in Velasco, et al., incorporated herein by reference in its entirety, at day 23-27).

### Human-Mouse Chimeroids

Even though it has been shown that brain organoids can recapitulate human fetal development, the field still lacks a model that can be grown over extended periods of time and acquire mature features to study late-onset biology of the brain, such as neurodegenerative diseases. Interestingly, the tempo of development of organoids is highly constrained within species, and such species-specific differences hold true in culture. As an example, human neurons develop more slowly than mice in a variety of systems, from 2D cultures to 3D brain organoids.

Taking advantage of the new model of human-mouse cortical chimeroids described herein, in which both species co-exist and develop within the same organoid, methods and tools for mechanistic understanding of fundamental biological processes that involve the species-specific tempo of development become available for the first time. The inventors decoded which non-cell autonomous mechanisms and molecules influence human cortical development dynamics and, thus, establish novel avenues to accelerate human neuron maturation in vitro, a crucial step to advancing investigation of late-onset psychiatric diseases in older organoids. Accelerating the speed of development of human organoids will move the field forward meaningfully by enabling investigation of more mature properties of the brain.

The present invention provides methods for the in vitro generation of a 3-D neural tissue structure that is capable of sensory perception. In certain embodiments the neural tissue structure is also capable of response to sensory stimulation. In one embodiment, the brain organoid includes neurons of different brain regions (e.g., cortical and subcortical neurons), produced from the in vitro differentiation of cells (e.g., human cells such as human stem cells or other pluripotent, multipotent or totipotent cells). The generated organoid forms and comprises complex neural network circuits, which contain sensory cells. The network circuits include, but are not limited to, cortical neurons, subcortical neurons and functional sensory cells such as photoreceptors, olfactory cells, and other functional sensory cells (e.g., tactile sensory cells, etc.). The network circuits may display spontaneous activity, as well as stimulus-induced activity.

### Multi-Cell Chimeroids

Described herein is the generation of organoids in which brain cells of one brain region are mixed with cells originating outside that region and also outside the brain (e.g., microglia, photoreceptors of the retina, cell of the vasculature, and the like). This approach enables integration in a single brain organoid of cells that would not spontaneously emerge following integration of cells that permit sensing of information from the environment, such as photoreceptors for sensing light. In some aspects, light could then be used to stimulate circuit maturation in organoids and to record circuit responses to external sensory input in the context of disease. For example, circuits generated from cells of neuropsychiatric patients (or Autism patients) could be examined to determine if they would respond differently to sensory stimulation. In some aspects, light or sound could also be used as the necessary input to generate human brain organoid-machine interfaces.

The present invention provides methods for the in vitro generation of a chimeric organoid, where the chimeric organoid comprises cells from at least one brain region and cells from at least one region outside the brain. In some aspects, cells from outside the brain may include microglia, photoreceptors of the retina, and cells of the vasculature. In some aspects, cells are obtained from the pallial, subpallial, cerebral cortex, or ventral forebrain regions of the brain. In some aspects, cells from different regions are provided at a 1:1:1 ratio. In other aspects, cells from different regions are provided at a variable ratio.

### Embryologically Diverse Chimeroids

Described herein is the generation of organoids in which cells from different embryological origins are mixed together to recapitulate fundamental brain-body axes (e.g., endothelial, cardiac, gut cells, and the like). In some aspects, this organoid enables the investigation of more complex biology along different brain-body axes (e.g., endothelial, cardiac, gut cells, and the like). In some aspects, this organoid enables the investigation of more complex biology along different brain-body axes in the same organoid. The brain receives signals from other tissues in the body and reciprocates with signals from the brain to the other tissues (e.g., the gut-brain axes).

The present invention provides methods for the in vitro generation of a chimeric organoid, where the chimeric organoid comprises cells from a first embryological origin and cells from a second embryological origin. In some aspects, the chimeric organoid may further comprise cells from a third, fourth, fifth, sixth, etc., embryological origin. Cells from different embryological origin may include endothelial cells, cardiac cells, gut cells, and the like. In some aspects, cells from different embryological origins are provided at a 1:1:1 ratio. In other aspects, cells from different embryological origins are provided at a variable ratio.

### Uses of Chimeric Organoids

In some embodiments, the invention provides a method of screening for neurodevelopmental abnormalities. In some aspects, chimeroids described herein are used. In some embodiments, chimeroids are used as a model for disease risk factors (e.g., genetic variants or neurotoxic insults). For example, chimeroids may be contacted with one or more neurotoxic agents and changes to the chimeroids may be assessed. Changes across multiple cell types within the chimeroids may be assessed, as well as assessing donor-specific differences. In some aspects, a neurotoxic agent is ethanol (EtOH) or valproic acid (VPA). Chimeroids may be used to measure genetic variation in biological response in brain tissues of different subjects.

In some embodiments, the invention provides a method of screening test agents to identify candidate neurologically active agents. In some aspects, chimeroids described herein are used. In some embodiments, chimeroids are generated from cells derived from a subject having a neurological disease or disorder (e.g., a neurodegenerative disorder, such as epilepsy, autism spectrum disorder (ASD), bipolar disorder, schizophrenia or a neurological, neuropsychological, neuropsychiatric, neurodegenerative, or neuropsychopharmacological disease).

Some embodiments are directed to a method of screening for a candidate neurologically active agent, comprising contacting a chimeroid as described herein with a test agent, and assessing changes to the chimeroid, wherein the test agent is identified as a candidate neurologically active agent when contact with the test agent causes a change to the chimeroid as compared to a control chimeroid.

In some embodiments, one or more parameters of the chimeroid are measured in response to contact with the test agent. In some embodiments, growth, viability, cellular composition, developmental trajectory, or morphology of the contacted chimeroid is measured and compared to a control after contact with the test agent.

In some embodiments, the control chimeroid is a chimeroid not contacted with the test agent. In some embodiments, the test chimeroid is derived from a subject having a neurological disease or condition and the control chimeroid is from a subject without the neurological disease or condition. Thus, in certain embodiments, if contact of the test chimeroid with the test agent results in the test chimeroid exhibiting a property more similar to the control chimeroid, then the agent can be identified as a beneficial agent for the neurological disease or condition.

In some embodiments, a high throughput screen (HTS) is performed. A high throughput screen can utilize the highly reproducible chimeroids described herein. High throughput screens often involve testing large numbers of compounds with high efficiency, e.g., in parallel. Often such screening is performed in multiwell plates other vessels in which multiple physically separated cavities or depressions are present in a substrate. High throughput screens often involve use of automation, e.g., for liquid handling, imaging, data acquisition and processing, etc. Certain general principles and techniques that may be applied in embodiments of a HTS of the present invention are described in Macarrón R & Hertzberg RP. Design and implementation of high-throughput screening assays. Methods Mol Biol., 565:1-32, 2009 and/or An W F & Tolliday N J., Introduction: cell-based assays for high-throughput screening. Methods Mol Biol. 486:1-12, 2009, and/or references in either of these. Useful methods are also disclosed in High Throughput Screening: Methods and Protocols (Methods in Molecular Biology) by William P. Janzen (2002) and High-Throughput Screening in Drug Discovery (Methods and Principles in Medicinal Chemistry) (2006) by Jorg Hüser.

### Biobanking

Also disclosed herein are methods to freeze and thaw human neural stem cells and neural progenitor cells derived from human pluripotent stem cells amenable to the generation of human brain chimeroids. The methods enable acceleration of protocols for production of chimeroids by enabling the generation of frozen biobanks of human neural stem cells and neural progenitor cells derived from human pluripotent stem cells skipping the production steps of neural stem cells and neural progenitor cells derived from human pluripotent stem cells for each chimeroid differentiation procedure. The protocol also improves the reproducibility and reduces costs of chimeroid differentiation protocols by reducing the number of experiments through which neural stem cells and neural progenitor cells are produced from human pluripotent stem cells. An exemplary protocol for freezing the cells is provided in Example 4.

### Definitions

For convenience, certain terms employed herein, in the specification, examples and appended claims are collected here. Unless otherwise defined, all technical and scientific terms used herein have the same meaning as commonly understood by one of ordinary skill in the art to which this invention belongs.

The term “differentiated cell” is meant to include any primary cell that is not, in its native form, pluripotent as that term is defined herein nor is a neural stem cell or progenitor cell. Stated another way, the term “differentiated cell” refers to a cell of a more specialized cell type derived from a cell of a less specialized cell type (e.g., a stem cell such as an induced pluripotent stem cell) in a cellular differentiation process.

As used herein, the term “somatic cell” refers to any cells forming the body of an organism, as opposed to germline cells. In mammals, germline cells (also known as “gametes”) are the spermatozoa and ova which fuse during fertilization to produce a cell called a zygote, from which the entire mammalian embryo develops. Every other cell type in the mammalian body-apart from the sperm and ova, the cells from which they are made (gametocytes) and undifferentiated stem cells—is a somatic cell type: internal organs, skin, bones, blood, and connective tissue are all made up of somatic cells. In some embodiments the somatic cell is a “non-embryonic somatic cell,” by which is meant a somatic cell that is not present in or obtained from an embryo and does not result from proliferation of such a cell in vitro. In some embodiments the somatic cell is an “adult somatic cell” by which is meant a cell that is present in or obtained from an organism other than an embryo or a fetus or a juvenile or results from proliferation and differentiation of such a cell in vitro.

As used herein, the term “adult cell” refers to a cell found throughout the body after embryonic and juvenile development.

The term “progenitor” or “precursor” cell are used interchangeably herein and refer to cells that have a cellular phenotype that is more primitive (i.e., is at an earlier step along a developmental pathway or progression than is a fully differentiated cell) relative to a cell which it can give rise to by differentiation. Often, progenitor cells also have significant or very high proliferative potential. Progenitor cells can give rise to multiple distinct differentiated cell types or to a single differentiated cell type, depending on the developmental pathway and on the environment in which the cells develop and differentiate.

The term “organoid” refers to a three-dimensional organ-bud grown in vitro and in isolation from an intact organism. Organoids may be derived from stem cells (e.g., embryonic stem cells, induced pluripotent stem cells, patient-derived stem cells, tissue-specific stem cells such as neural stem cells, etc.). Various organoids may be formed including, but not limited to, brain organoids such as cerebral organoids and rostral organoids, thyroid organoids, intestinal organoids, testicular organoids, hepatic organoids, pancreatic organoids, gastric organoids, epithelial organoids, lung organoids, kidney organoids, retina organoids, inner ear organoids, and pituitary organoids.

The terms “cerebral organoid,” “3D brain tissue,” “cortical organoids,” or “brain organoid” are used interchangeably herein and refer to organoids that have anatomical features that resemble mammalian brains. The cerebral organoids may include synthesized tissues that contain several types of nerve cells. Cerebral organoids can be produced using human pluripotent stem cells (hPSCs). The general art-recognized methodology for producing cerebral organoids includes culturing hPSCs under conditions suitable for the development of an embryoid body. The cell culture/embryoid bodies are then patterned/guided towards neuroectoderm using small molecules. The neuroectoderm begins to proliferate and grow and is transferred to a tissue culture vessel where the cerebral organoids will continue to develop. Brain organoids may differentiate into one or more of various neural tissue types, such as the optic cup, hippocampus, ventral parts of the telencephalon and dorsal cortex.

The terms “chimeric brain organoid,” “multi-donor chimeroid,” and “brain chimeroid” are used interchangeably to refer to brain organoids derived from cells (e.g., stem cells) obtained from or derived from multiple sources, also termed cells of chimeric origin herein. For example, the stem cells can be derived from multiple individuals within the same species, e.g., from multiple humans or from multiple mice, resulting in a “pool” of individual genetic backgrounds within the same organoid. Alternatively, the stem cells can be derived from individuals from different species; for example, a brain chimeroid can be prepared from a combination of stem cells from both one or more humans and one or more rodents (e.g., mice). In additional embodiments the cells can be derived from different regions of the brain, for example from cells of pallial and subpallial identity which typically originate in distinct areas of the normal brain as opposed to coming from the same region of the brain.

The term “embryoid bodies” is synonymous with “aggregate bodies.” The terms refer to aggregates of undifferentiated (but patterned) cells, such as tissue specific stem cells that may or may not derived from PSC (pluripotent stem cells).

The term “phenotype” refers to one or a number of total biological characteristics that define the cell or organism under a particular set of environmental conditions and factors, regardless of the actual genotype.

The term “pluripotent” as used herein refers to a cell with the capacity to differentiate to more than one differentiated cell type, and preferably to differentiate to cell types characteristic of all three germ cell layers. Pluripotent cells are characterized primarily by their ability to differentiate to more than one cell type, preferably to all three germ layers, using, for example, a nude mouse teratoma formation assay. Pluripotency is also evidenced by the expression of embryonic stem (ES) cell markers, although the preferred test for pluripotency is the demonstration of the capacity to differentiate into cells of each of the three germ layers. It should be noted that simply culturing such cells does not, on its own, render them pluripotent. Reprogrammed pluripotent cells (e.g., iPS cells as that term is defined herein) also have the characteristic of the capacity of extended passaging without loss of growth potential, relative to primary cell parents, which generally have capacity for only a limited number of divisions in culture.

As used herein, the terms “iPS cell” and “induced pluripotent stem cell” are used interchangeably and refers to a pluripotent stem cell artificially derived (e.g., induced or by complete reversal) from a non-pluripotent cell, typically an adult somatic cell, for example, by inducing a forced expression of one or more genes.

The term “stem cell” as used herein, refers to an undifferentiated cell which is capable of proliferation and giving rise to more progenitor cells having the ability to generate a large number of differentiated cells that can in turn give rise to more differentiated, or differentiable daughter cells. The daughter cells themselves can be induced to proliferate and produce progeny that subsequently differentiate into one or more mature cell types, while also retaining one or more cells with parental developmental potential. The term “stem cell” refers to a subset of progenitors that have the capacity or potential, under particular circumstances, to differentiate to a more specialized or differentiated phenotype, and which retains the capacity, under certain circumstances, to proliferate without substantially differentiating. In one embodiment, the term stem cell refers generally to a naturally occurring mother cell whose descendants (progeny) specialize, often in different directions, by differentiation, e.g., by acquiring completely individual characters, as occurs in progressive diversification of embryonic cells and tissues. Cellular differentiation is a complex process typically occurring through many cell divisions. A differentiated cell may derive from a multipotent cell which itself is derived from a multipotent cell, and so on. While each of these multipotent cells may be considered stem cells, the range of cell types each can give rise to may vary considerably. Some differentiated cells also have the capacity to give rise to cells of greater developmental potential. Such capacity may be natural or may be induced artificially upon treatment with various factors. In many biological instances, stem cells are also “multipotent” because they can produce progeny of more than one distinct cell type, but this is not required for “stem-ness.” Self-renewal is the other classical part of the stem cell definition, and it is essential as used in this document. In theory, self-renewal can occur by either of two major mechanisms. Stem cells may divide asymmetrically, with one daughter retaining the stem state and the other daughter expressing some distinct other specific function and phenotype. Alternatively, some of the stem cells in a population can divide symmetrically into two stems, thus maintaining some stem cells in the population as a whole, while other cells in the population give rise to differentiated progeny only. Formally, it is possible that cells that begin as stem cells might proceed toward a differentiated phenotype, but then “reverse” and re-express the stem cell phenotype, a term often referred to as “dedifferentiation” or “reprogramming” or “retrodifferentiation” by persons of ordinary skill in the art. As used herein, the term “pluripotent stem cell” includes embryonic stem cells, induced pluripotent stem cells, placental stem cells, etc.

In the context of cell ontogeny, the adjective “differentiated”, or “differentiating” is a relative term meaning a “differentiated cell” is a cell that has progressed further down the developmental pathway than the cell it is being compared with. Thus, stem cells can differentiate to lineage-restricted precursor cells (such as an ectodermal stem cell), which in turn can differentiate into other types of precursor cells further down the pathway (such as a neural ectodermal cell), and then to an end-stage differentiated cell, which plays a characteristic role in a certain tissue type, and may or may not retain the capacity to proliferate further.

The term “embryonic stem cell” is used to refer to the pluripotent stem cells of the inner cell mass of the embryonic blastocyst (see U.S. Pat. Nos. 5,843,780, 6,200,806). Such cells can similarly be obtained from the inner cell mass of blastocysts derived from somatic cell nuclear transfer (see, for example, U.S. Pat. Nos. 5,945,577, 5,994,619, 6,235,970). The distinguishing characteristics of an embryonic stem cell define an embryonic stem cell phenotype. Accordingly, a cell has the phenotype of an embryonic stem cell if it possesses one or more of the unique characteristics of an embryonic stem cell such that that cell can be distinguished from other cells. Exemplary distinguishing embryonic stem cell characteristics include, without limitation, gene expression profile, proliferative capacity, differentiation capacity, karyotype, responsiveness to particular culture conditions, and the like.

The term “adult stem cell” or “ASC” is used to refer to any multipotent stem cell derived from non-embryonic tissue, including fetal, juvenile, and adult tissue. Stem cells have been isolated from a wide variety of adult tissues including blood, bone marrow, brain, olfactory epithelium, skin, pancreas, skeletal muscle, and cardiac muscle. Each of these stem cells can be characterized based on gene expression, factor responsiveness, and morphology in culture. Exemplary adult stem cells include adult neural stem cells, neural crest stem cells, mesenchymal stem cells, hematopoietic stem cells, and pancreatic stem cells. As indicated above, stem cells have been found resident in virtually every tissue. Accordingly, the present invention appreciates that stem cell populations can be isolated from virtually any animal tissue.

The term “reprogramming” as used herein refers to the process that alters or reverses the differentiation state of a somatic cell. The cell can either be partially or terminally differentiated prior to the reprogramming. Reprogramming encompasses complete reversion of the differentiation state of a somatic cell to a pluripotent cell. Such complete reversal of differentiation produces an induced pluripotent (iPS) cell. Reprogramming as used herein also encompasses partial reversion of a cells differentiation state, for example to a multipotent state or to a somatic cell that is neither pluripotent or multipotent, but is a cell that has lost one or more specific characteristics of the differentiated cell from which it arises, e.g. direct reprogramming of a differentiated cell to a different somatic cell type. Reprogramming generally involves alteration, e.g., reversal, of at least some of the heritable patterns of nucleic acid modification (e.g., methylation), chromatin condensation, epigenetic changes, genomic imprinting, etc., that occur during cellular differentiation as a zygote develops into an adult.

The term “agent” as used herein means any compound or substance such as, but not limited to, a small molecule, nucleic acid, polypeptide, peptide, drug, ion, etc. An “agent” can be any chemical, entity or moiety, including without limitation synthetic and naturally-occurring proteinaceous and non-proteinaceous entities. In some embodiments, an agent is nucleic acid, nucleic acid analogues, proteins, antibodies, peptides, aptamers, oligomer of nucleic acids, amino acids, or carbohydrates including without limitation proteins, oligonucleotides, ribozymes, DNAzymes, glycoproteins, siRNAs, lipoproteins, aptamers, and modifications and combinations thereof etc. In certain embodiments, agents are small molecules having a chemical moiety. For example, chemical moieties include unsubstituted or substituted alkyl, aromatic, or heterocyclyl moieties including macrolides, leptomycins and related natural products or analogues thereof. Compounds can be known to have a desired activity and/or property or can be selected from a library of diverse compounds.

As used herein, the term “contacting” is intended to include incubating the agent, medium or cell, with another agent, medium or cell together in vitro (e.g., adding a differentiation medium or agent to cells in culture). In some embodiments, the term “contacting” is not intended to include the in vivo exposure of cells to the compounds as disclosed herein that may occur naturally in a subject (i.e., exposure that may occur as a result of a natural physiological process). A step of contacting can be conducted in any suitable manner. For example, cells may be treated in adherent culture or in suspension culture. In some embodiments, cells are treated in conditions that promote cell clustering, including, without limitation, suspension culture in low attachment tissue culture plates, spinner flasks, aggrewell plates

The term “cell culture medium” (also referred to herein as a “culture medium” or “medium”) as referred to herein is a medium for culturing cells containing nutrients that maintain cell viability and support proliferation. The cell culture medium may contain any of the following in an appropriate combination: salt(s), buffer(s), amino acids, glucose or other sugar(s), antibiotics, serum or serum replacement, and other components such as peptide growth factors, etc. Cell culture media ordinarily used for particular cell types are known to those skilled in the art and can be supplemented with additional agents to achieve the purposes described herein.

The term “cell line” refers to a population of largely or substantially identical cells that has typically been derived from a single ancestor cell or from a defined and/or substantially identical population of ancestor cells. The cell line may have been or may be capable of being maintained in culture for an extended period (e.g., months, years, for an unlimited period of time). It may have undergone a spontaneous or induced process of transformation conferring an unlimited culture lifespan on the cells. Cell lines include all those cell lines recognized in the art as such. It will be appreciated that cells acquire mutations and possibly epigenetic changes over time such that at least some properties of individual cells of a cell line may differ with respect to each other. In some embodiments, a cell line comprises a neural cell or neural tissue described herein.

The terms “feeder cells” or “feeders” refer to cells of one type that are co-cultured with cells of another type, to provide an environment in which the cells of the second type can grow. The feeder cells are optionally from a different species as the cells they are supporting. In some aspects, a culture or cell population may be referred to as “feeder free”, meaning the composition is essentially free of feeder cells.

The term “growth environment” refers to an environment in which cells of interest will proliferate or differentiate in vitro. Features of the environment include the medium in which the cells are cultured, the temperature, the partial pressure of O₂ and CO₂, and a supporting structure (such as a substrate on a solid surface) if present.

The term “nutrient medium” refers to a medium for culturing cells containing nutrients that promote proliferation. The nutrient medium may contain any of the following in an appropriate combination: isotonic saline, buffer, amino acids, antibiotics, serum or serum replacement, and exogenously added factors. A “conditioned medium” is prepared by culturing a first population of cells in a medium, and then harvesting the medium. The conditioned medium (along with anything secreted into the medium by the cells) may then be used to support the growth of a second population of cells.

The term “exogenous” refers to a substance present in a cell or organism other than its native source. For example, the terms “exogenous nucleic acid” or “exogenous protein” refer to a nucleic acid or protein that has been introduced by a process involving the hand of man into a biological system such as a cell or organism in which it is not normally found or in which it is found in lower amounts. A substance will be considered exogenous if it is introduced into a cell or an ancestor of the cell that inherits the substance. In contrast, the term “endogenous” refers to a substance that is native to the biological system.

The term “expression” refers to the cellular processes involved in producing RNA and proteins and as appropriate, secreting proteins, including where applicable, but not limited to, transcription, translation, folding, modification and processing. “Expression products” include RNA transcribed from a gene and polypeptides obtained by translation of mRNA transcribed from a gene.

The terms “genetically modified” or “engineered” cell as used herein refers to a cell into which an exogenous nucleic acid has been introduced by a process involving the hand of man (or a descendant of such a cell that has inherited at least a portion of the nucleic acid). The nucleic acid may for example contain a sequence that is exogenous to the cell, it may contain native sequences (i.e., sequences naturally found in the cells) but in a non-naturally occurring arrangement (e.g., a coding region linked to a promoter from a different gene), or altered versions of native sequences, etc. The process of transferring the nucleic acid into the cell can be achieved by any suitable technique. Suitable techniques include calcium phosphate or lipid-mediated transfection, electroporation, and transduction or infection using a viral vector. In some embodiments the polynucleotide or a portion thereof is integrated into the genome of the cell. The nucleic acid may have subsequently been removed or excised from the genome, provided that such removal or excision results in a detectable alteration in the cell relative to an unmodified but otherwise equivalent cell. It should be appreciated that the term genetically modified is intended to include the introduction of a modified RNA directly into a cell (e.g., a synthetic, modified RNA). Such synthetic modified RNAs include modifications to prevent rapid degradation by endo- and exo-nucleases and to avoid or reduce the cell's innate immune or interferon response to the RNA. Modifications include, but are not limited to, (a) end modifications, e.g., 5′ end modifications (phosphorylation dephosphorylation, conjugation, inverted linkages, etc.) or 3′ end modifications (conjugation, DNA nucleotides, inverted linkages, etc.); (b) base modifications, e.g., replacement with modified bases, stabilizing bases, destabilizing bases, bases that base pair with an expanded repertoire of partners, or conjugated bases; (c) sugar modifications (e.g., at the 2′ position or 4′ position) or replacement of the sugar; and (d) internucleoside linkage modifications, including modification or replacement of the phosphodiester linkages. To the extent that such modifications interfere with translation, the modification is not suitable for the methods and compositions described herein.

The term “isolated” or “partially purified” as used herein refers, in the case of a nucleic acid or polypeptide, to a nucleic acid or polypeptide separated from at least one other component (e.g., nucleic acid or polypeptide) that is present with the nucleic acid or polypeptide as found in its natural source and/or that would be present with the nucleic acid or polypeptide when expressed by a cell, or secreted in the case of secreted polypeptides. A chemically synthesized nucleic acid or polypeptide or one synthesized using in vitro transcription/translation is considered “isolated”.

The term “isolated cell” as used herein refers to a cell that has been removed from an organism in which it was originally found or a descendant of such a cell. Optionally the cell has been cultured in vitro, e.g., in the presence of other cells. Optionally the cell is later introduced into a second organism or re-introduced into the organism from which it (or the cell from which it is descended) was isolated.

The term “isolated population” with respect to an isolated population of cells as used herein refers to a population of cells that has been removed and separated from a mixed or heterogeneous population of cells. In some embodiments, an isolated population is a substantially pure population of cells as compared to the heterogeneous population from which the cells were isolated or enriched from.

The terms “enriching” or “enriched” are used interchangeably herein and mean that the yield (fraction) of cells of one type is increased by at least 10% over the fraction of cells of that type in the starting culture or preparation.

The terms “renewal” or “self-renewal” or “proliferation” are used interchangeably herein, to refer to the ability of stem cells to renew themselves by dividing into the same non-specialized cell type over long periods (e.g., over many months to years). In some instances, proliferation refers to the expansion of cells by the repeated division of single cells into two identical daughter cells.

The term “lineages” as used herein describes a cell with a common ancestry or cells with a common developmental fate. For example, in the context of a cell that is of ectoderm origin or is “ectodermal linage” this means the cell was derived from an ectoderm cell and can differentiate along the ectoderm lineage restricted pathways.

As used herein, the term “xenogeneic” refers to cells that are derived from different species.

The term “modulate” is used consistently with its use in the art, i.e., meaning to cause or facilitate a qualitative or quantitative change, alteration, or modification in a process, pathway, or phenomenon of interest. Without limitation, such change may be an increase, decrease, or change in relative strength or activity of different components or branches of the process, pathway, or phenomenon. A “modulator” is an agent that causes or facilitates a qualitative or quantitative change, alteration, or modification in a process, pathway, or phenomenon of interest.

The term “polynucleotide” is used herein interchangeably with “nucleic acid” to indicate a polymer of nucleosides. Typically a polynucleotide of this invention is composed of nucleosides that are naturally found in DNA or RNA (e.g., adenosine, thymidine, guanosine, cytidine, uridine, deoxyadenosine, deoxythymidine, deoxyguanosine, and deoxycytidine) joined by phosphodiester bonds. However the term encompasses molecules comprising nucleosides or nucleoside analogs containing chemically or biologically modified bases, modified backbones, etc., whether or not found in naturally occurring nucleic acids, and such molecules may be preferred for certain applications. Where this application refers to a polynucleotide it is understood that both DNA, RNA, and in each case both single- and double-stranded forms (and complements of each single-stranded molecule) are provided. “Polynucleotide sequence” as used herein can refer to the polynucleotide material itself and/or to the sequence information (i.e. the succession of letters used as abbreviations for bases) that biochemically characterizes a specific nucleic acid. A polynucleotide sequence presented herein is presented in a 5′ to 3′ direction unless otherwise indicated.

The term “polypeptide” as used herein refers to a polymer of amino acids. The terms “protein” and “polypeptide” are used interchangeably herein. A peptide is a relatively short polypeptide, typically between about 2 and 60 amino acids in length. Polypeptides used herein typically contain amino acids such as the 20 L-amino acids that are most commonly found in proteins. However, other amino acids and/or amino acid analogs known in the art can be used. One or more of the amino acids in a polypeptide may be modified, for example, by the addition of a chemical entity such as a carbohydrate group, a phosphate group, a fatty acid group, a linker for conjugation, functionalization, etc. A polypeptide that has a non-polypeptide moiety covalently or non-covalently associated therewith is still considered a “polypeptide”. Exemplary modifications include glycosylation and palmitoylation. Polypeptides may be purified from natural sources, produced using recombinant DNA technology, synthesized through chemical means such as conventional solid phase peptide synthesis, etc. The term “polypeptide sequence” or “amino acid sequence” as used herein can refer to the polypeptide material itself and/or to the sequence information (i.e., the succession of letters or three letter codes used as abbreviations for amino acid names) that biochemically characterizes a polypeptide. A polypeptide sequence presented herein is presented in an N-terminal to C-terminal direction unless otherwise indicated.

The term “functional fragments” as used herein is a polypeptide having amino acid sequence which is smaller in size than, but substantially homologous to the polypeptide it is a fragment of, and where the functional fragment polypeptide sequence is about at least 50%, or 60% or 70% or 80% or 90% or 100% or greater than 100%, for example 1.5-fold, 2-fold, 3-fold, 4-fold or greater than 4-fold effective biological action as the polypeptide from which it is a fragment of. Functional fragment polypeptides may have additional functions that can include decreased antigenicity, increased DNA binding (as in transcription factors), or altered RNA binding (as in regulating RNA stability or degradation).

A “marker” as used herein is used to describe the characteristics and/or phenotype of a cell. Markers can be used for selection of cells comprising characteristics of interests. Markers will vary with specific cells. Markers are characteristics, whether morphological, functional or biochemical (enzymatic) characteristics of the cell of a particular cell type, or molecules expressed by the cell type. Preferably, such markers are proteins, and more preferably, possess an epitope for antibodies or other binding molecules available in the art. However, a marker may consist of any molecule found in a cell including, but not limited to, proteins (peptides and polypeptides), lipids, polysaccharides, nucleic acids and steroids. Examples of morphological characteristics or traits include, but are not limited to, shape, size, and nuclear to cytoplasmic ratio. Examples of functional characteristics or traits include, but are not limited to, the ability to adhere to particular substrates, ability to incorporate or exclude particular dyes, ability to migrate under particular conditions, and the ability to differentiate along particular lineages. Markers may be detected by any method available to one of skill in the art. Markers can also be the absence of a morphological characteristic or absence of proteins, lipids etc. Markers can be a combination of a panel of unique characteristics of the presence and absence of polypeptides and other morphological characteristics.

The term “selectable marker” refers to a gene, RNA, or protein that when expressed, confers upon cells a selectable phenotype, such as resistance to a cytotoxic or cytostatic agent (e.g., antibiotic resistance), nutritional prototrophy, or expression of a particular protein that can be used as a basis to distinguish cells that express the protein from cells that do not. Proteins whose expression can be readily detected such as a fluorescent or luminescent protein or an enzyme that acts on a substrate to produce a colored, fluorescent, or luminescent substance (“detectable markers”) constitute a subset of selectable markers. The presence of a selectable marker linked to expression control elements native to a gene that is normally expressed selectively or exclusively in pluripotent cells makes it possible to identify and select somatic cells that have been reprogrammed to a pluripotent state. A variety of selectable marker genes can be used, such as neomycin resistance gene (neo), puromycin resistance gene (puro), guanine phosphoribosyl transferase (gpt), dihydrofolate reductase (DHFR), adenosine deaminase (ada), puromycin-N-acetyltransferase (PAC), hygromycin resistance gene (hyg), multidrug resistance gene (mdr), thymidine kinase (TK), hypoxanthine-guanine phosphoribosyltransferase (HPRT), and hisD gene. Detectable markers include green fluorescent protein (GFP) blue, sapphire, yellow, red, orange, and cyan fluorescent proteins and variants of any of these. Luminescent proteins such as luciferase (e.g., firefly or Renilla luciferase) are also of use. As will be evident to one of skill in the art, the term “selectable marker” as used herein can refer to a gene or to an expression product of the gene, e.g., an encoded protein.

In some embodiments the selectable marker confers a proliferation and/or survival advantage on cells that express it relative to cells that do not express it or that express it at significantly lower levels. Such proliferation and/or survival advantage typically occurs when the cells are maintained under certain conditions, i.e., “selective conditions.” To ensure an effective selection, a population of cells can be maintained under conditions and for a sufficient period of time such that cells that do not express the marker do not proliferate and/or do not survive and are eliminated from the population or their number is reduced to only a very small fraction of the population. The process of selecting cells that express a marker that confers a proliferation and/or survival advantage by maintaining a population of cells under selective conditions so as to largely or completely eliminate cells that do not express the marker is referred to herein as “positive selection”, and the marker is said to be “useful for positive selection”. Negative selection and markers useful for negative selection are also of interest in certain of the methods described herein. Expression of such markers confers a proliferation and/or survival disadvantage on cells that express the marker relative to cells that do not express the marker or express it at significantly lower levels (or, considered another way, cells that do not express the marker have a proliferation and/or survival advantage relative to cells that express the marker). Cells that express the marker can therefore be largely or completely eliminated from a population of cells when maintained in selective conditions for a sufficient period of time.

The terms “subject” and “individual” are used interchangeably herein, and refer to an animal, for example, a human from whom cells can be obtained and/or to whom treatment, including prophylactic treatment, with the cells as described herein, is provided. For treatment of those infections, conditions or disease states which are specific for a specific animal such as a human subject, the term subject refers to that specific animal. The terms “non-human animals” and “non-human mammals” as used herein interchangeably, includes mammals such as rats, mice, rabbits, sheep, cats, dogs, cows, pigs, and non-human primates. The term “subject” also encompasses any vertebrate including but not limited to mammals, reptiles, amphibians and fish. However, advantageously, the subject is a mammal such as a human, or other mammals such as a domesticated mammal, e.g. dog, cat, horse, and the like, or production mammal, e.g. cow, sheep, pig, and the like.

The terms “treat”, “treating”, “treatment”, etc., as applied to an isolated cell, include subjecting the cell to any kind of process or condition or performing any kind of manipulation or procedure on the cell. As applied to a subject, the terms refer to providing medical or surgical attention, care, or management to an individual. The individual is usually ill or injured, or at increased risk of becoming ill relative to an average member of the population and in need of such attention, care, or management.

The terms “treating” and “treatment” refer to administering to a subject an effective amount of a composition so that the subject experiences a reduction in at least one symptom of the disease or an improvement in the disease, for example, beneficial or desired clinical results. For purposes of this invention, beneficial or desired clinical results include, but are not limited to, alleviation of one or more symptoms, diminishment of extent of disease, stabilized (i.e., not worsening) state of disease, delay or slowing of disease progression, amelioration or palliation of the disease state, and remission (whether partial or total), whether detectable or undetectable. Treating can refer to prolonging survival as compared to expected survival if not receiving treatment. Thus, one of skill in the art realizes that a treatment may improve the disease condition, but may not be a complete cure for the disease. As used herein, the term “treatment” includes prophylaxis. Alternatively, treatment is “effective” if the progression of a disease is reduced or halted. “Treatment” can also mean prolonging survival as compared to expected survival if not receiving treatment. Those in need of treatment include those already diagnosed with a cardiac condition, as well as those likely to develop a cardiac condition due to genetic susceptibility or other factors such as weight, diet and health.

The term “test compound” refers to any of a small molecule, nucleic acid, amino acid, polypeptide, antibody and antibody-like molecules, aptamers, macrocycles, or other molecules. In certain embodiments, a test compound is a small organic molecule. In one aspect of these embodiments, the small organic molecule has a molecular weight of less than about 5,000 daltons. In certain embodiments, the test compound is other than an amino acid. In other embodiments, the small molecule is other than leucine, isoleucine or analogs of either of the foregoing.

As used herein, “neuro disorder” or “neuro disease” refer to neurodegenerative disorders, neuropsychiatric disorders and/or neurodevelopmental disorders. “Neuro disease” also refers to neurological, neuropsychological, neuropsychiatric, neurodegenerative, or neuropsychopharmacological diseases. Neuro disorders may be any disease affecting neuronal network connectivity, synaptic function and activity. “Neurodegenerative disorder” refers to a disease condition involving neural loss mediated or characterized at least partially by at least one of deterioration of neural stem cells and/or progenitor cells. Non-limiting examples of neurodegenerative disorders include polyglutamine expansion disorders (e.g., HD, dentatorubropallidoluysian atrophy, Kennedy's disease (also referred to as spinobulbar muscular atrophy), and spinocerebellar ataxia (e.g., type 1, type 2, type 3 (also referred to as Machado-Joseph disease), type 6, type 7, and type 17), other trinucleotide repeat expansion disorders (e.g., fragile X syndrome, fragile XE mental retardation, Friedreich's ataxia, myotonic dystrophy, spinocerebellar ataxia type 8, and spinocerebellar ataxia type 12), Alexander disease, Alper's disease, Alzheimer disease, amyotrophic lateral sclerosis (ALS), ataxia telangiectasia, Batten disease (also referred to as Spielmeyer-Vogt-Sjogren-Batten disease), Canavan disease, Cockayne syndrome, corticobasal degeneration, Creutzfeldt-Jakob disease, Guillain-Barré syndrome, ischemia stroke, Krabbe disease, kuru, Lewy body dementia, multiple sclerosis, multiple system atrophy, non-Huntingtonian type of Chorea, Parkinson's disease, Pelizaeus-Merzbacher disease, Pick's disease, primary lateral sclerosis, progressive supranuclear palsy, Refsum's disease, Sandhoff disease, Schilder's disease, spinal cord injury, spinal muscular atrophy (SMA), SteeleRichardson-Olszewski disease, and Tabes dorsalis.

In certain contexts, neurodegenerative disorders encompass neurological injuries or damages to the CNS or the PNS associated with physical injury (e.g., head trauma, mild to severe traumatic brain injury (TBI), spinal cord injury, diffuse axonal injury, craniocerebral trauma, cranial nerve injuries, cerebral contusion, intracerebral haemorrhage and acute brain swelling), ischemia (e.g., resulting from spinal cord infarction or ischemia, ischemic infarction, stroke, cardiac insufficiency or arrest, atherosclerotic thrombosis, ruptured aneurysm, embolism or hemorrhage), certain medical procedures or exposure to biological or chemic toxins or poisons (e.g., surgery, coronary artery bypass graft (CABG), electroconvulsive therapy, radiation therapy, chemotherapy, anti-neoplastic drugs, immunosuppressive agents, psychoactive, sedative or hypnotic drugs, alcohol, bacterial or industrial toxins, plant poisons, and venomous bites and stings), tumors (e.g., CNS metastasis, intraaxial tumors, primary CNS lymphomas, germ cell tumors, infiltrating and localized gliomas, fibrillary astrocytomas, oligodendrogliomas, ependymomas, pleomorphic xanthoastrocytomas, pilocytic astrocytomas, extraaxial brain tumors, meningiomas, schwannomas, neurofibromas, pituitary tumors, and mesenchymal tumors of the skull, spine and dura matter), infections (e.g., bacterial, viral, fungal, parasitic or other origin is selected from the group consisting of pyrogenic infections, meningitis, tuberculosis, syphilis, encephalomyelitis and leptomeningitis), metabolic or nutritional disorders (e.g., glycogen storage diseases, acid lipase diseases, Wemicke's or Marchiafava-Bignami's disease, Lesch-Nyhan syndrome, Farber's disease, gangliosidoses, vitamin B12 and folic acid deficiency), cognition or mood disorders (e.g., learning or memory disorder, bipolar disorders and depression), and various medical conditions associated with neural damage or destruction (e.g., asphyxia, prematurity in infants, perinatal distress, gaseous intoxication for instance from carbon monoxide or ammonia, coma, hypoglycaemia, dementia, epilepsy and hypertensive crises).

“Neurological disorder” encompasses mental disorders attributable to diseases of the nervous system. Non-limiting examples of neuropsychiatric disorders include addictions, childhood developmental disorders, eating disorders, degenerative diseases, mood disorders, neurotic disorders, psychosis, sleep disorders, depression, obsessive-compulsive disorder, schizophrenia, visual hallucination, auditory hallucination, eating disorder, bipolar disorder, epilepsy, autism spectrum disorder (ASD), and amyotrophic lateral sclerosis (ALS), and other neurodegenerative, neuropsychiatric and neurodevelopmental disorders.

The term “tissue” refers to a group or layer of specialized cells which together perform certain special functions. The term “tissue-specific” refers to a source of cells from a specific tissue.

The terms “decrease”, “reduced”, “reduction”, “decrease”, and “inhibit” are all used herein generally to mean a decrease by a statistically significant amount. However, for avoidance of doubt, “reduced”, “reduction” or “decrease” or “inhibit” means a decrease by at least 10% as compared to a reference level, for example a decrease by at least about 20%, or at least about 30%, or at least about 40%, or at least about 50%, or at least about 60%, or at least about 70%, or at least about 80%, or at least about 90% or up to and including a 100% decrease (i.e. absent level as compared to a reference sample), or any decrease between 10-100% as compared to a reference level.

The terms “increased”, “increase”, “enhance” or “activate” are all used herein to generally mean an increase by a statically significant amount; for the avoidance of any doubt, the terms “increased”, “increase”, “enhance” or “activate” means an increase of at least 10% as compared to a reference level, for example an increase of at least about 20%, or at least about 30%, or at least about 40%, or at least about 50%, or at least about 60%, or at least about 70%, or at least about 80%, or at least about 90% or up to and including a 100% increase or any increase between 10-100% as compared to a reference level, or at least about a 2-fold, or at least about a 3-fold, or at least about a 4-fold, or at least about a 5-fold or at least about a 10-fold increase, or any increase between 2-fold and 10-fold or greater as compared to a reference level.

The articles “a”, “an” and “the” as used herein, unless clearly indicated to the contrary, should be understood to include the plural referents. Claims or descriptions that include “or” between one or more members of a group are considered satisfied if one, more than one, or all of the group members are present in, employed in, or otherwise relevant to a given product or process unless indicated to the contrary or otherwise evident from the context. It should it be understood that, in general, where the invention, or aspects of the invention, is/are referred to as comprising particular elements, features, etc., certain embodiments of the invention or aspects of the invention consist, or consist essentially of, such elements, features, etc. For purposes of simplicity those embodiments have not in every case been specifically set forth in haec verba herein. It should also be understood that any embodiment of the invention, e.g., any embodiment found within the prior art, can be explicitly excluded from the claims, regardless of whether the specific exclusion is recited in the specification.

As used herein the term “comprising” or “comprises” is used in reference to compositions, methods, and respective component(s) thereof, that are essential to the invention, yet open to the inclusion of unspecified elements, whether essential or not. The term “consisting essentially of” refers to those elements required for a given embodiment. The term permits the presence of additional elements that do not materially affect the basic and novel or functional characteristic(s) of that embodiment of the invention. The term “consisting of” refers to compositions, methods, and respective components thereof as described herein, which are exclusive of any element not recited in that description of the embodiment.

Where ranges are given herein, the invention includes embodiments in which the endpoints are included, embodiments in which both endpoints are excluded, and embodiments in which one endpoint is included and the other is excluded. It should be assumed that both endpoints are included unless indicated otherwise. Furthermore, it is to be understood that unless otherwise indicated or otherwise evident from the context and understanding of one of skill in the art, values that are expressed as ranges can assume any specific value or subrange within the stated ranges in different embodiments of the invention, to the tenth of the unit of the lower limit of the range, unless the context clearly dictates otherwise. It is also understood that where a series of numerical values is stated herein, the invention includes embodiments that relate analogously to any intervening value or range defined by any two values in the series, and that the lowest value may be taken as a minimum and the greatest value may be taken as a maximum. Numerical values, as used herein, include values expressed as percentages. For any embodiment of the invention in which a numerical value is prefaced by “about” or “approximately”, the invention includes an embodiment in which the exact value is recited. For any embodiment of the invention in which a numerical value is not prefaced by “about” or “approximately”, the invention includes an embodiment in which the value is prefaced by “about” or “approximately”. “Approximately” or “about” generally includes numbers that fall within a range of 1% or in some embodiments 5% of a number in either direction (greater than or less than the number) unless otherwise stated or otherwise evident from the context (except where such number would impermissibly exceed 100% of a possible value).

Furthermore, it is to be understood that the invention encompasses all variations, combinations, and permutations in which one or more limitations, elements, clauses, descriptive terms, etc., from one or more of the listed claims is introduced into another claim dependent on the same base claim (or, as relevant, any other claim) unless otherwise indicated or unless it would be evident to one of ordinary skill in the art that a contradiction or inconsistency would arise. Where elements are presented as lists, e.g., in Markush group or similar format, it is to be understood that each subgroup of the elements is also disclosed, and any element(s) can be removed from the group.

Certain claims are presented in dependent form for the sake of convenience, but any dependent claim may be rewritten in independent format to include the limitations of the independent claim and any other claim(s) on which such claim depends, and such rewritten claim is to be considered equivalent in all respects to the dependent claim (either amended or unamended) prior to being rewritten in independent format. It should also be understood that, unless clearly indicated to the contrary, in any methods claimed herein that include more than one act, the order of the acts of the method is not necessarily limited to the order in which the acts of the method are recited, but the invention includes embodiments in which the order is so limited. It is contemplated that all aspects described above are applicable to all different embodiments of the invention. It is also contemplated that any of the above embodiments can be freely combined with one or more other such embodiments whenever appropriate.

### EXEMPLIFICATION

### Example 1

### Methods

### hPS Cell Culture

All human pluripotent stem (hPS) cell lines were cultured as previously described (Velasco et al., Nature June 570 (7762): 523-527 (2019); Paulsen, Velasco, Kedaigle and Pigoni et al., Nature February 602 (7896): 268-273 (2022)). MTESR1 medium (StemCell Technologies), mTESR+ medium (StemCell Technologies) or StemFlex medium (Gibco), all with 1% of added Penicillin-Streptomycin Solution (Corning), were used for culture of stem cells in previously coated cell culture dishes (Falcon) with 1% Geltrex (Gibco) with culture conditions of 37° C. in 5% CO₂. All hPS cell lines were maintained below passage 50, tested negative for Mycoplasma (assayed with MycoAlert PLUS Mycoplasma Detection Kit, Lonza) and were karyotypically normal (G-banded karyotype test performed by WiCell Research Institute).

### hPS Cell Lines

The female hESC HUES66 CHD8 AC2 clone was previously described ((Paulsen, Velasco, Kedaigle and Pigoni et al., Nature February 602 (7896): 268-273 (2022)). This clone has a heterozygous 13-nucleotide deletion, resulting in a stop codon at amino acid 1248 (CHD8 gRNA: 5′-TTCTTACTGTGTACCCGGGC-3′ (TGG); SEQ ID NO: 1), and was provided by N. Sanjana, X. Shi, J. Pan and F. Zhang (Broad Institute of MIT and Harvard). The psychiatric control Mito210 parental male iPS cell line was provided by B. Cohen (McLean Hospital); the parental PGP1 male iPS cell line by G. Church (Harvard University) (Church, G. M). The personal genome project. Mol. Syst. Biol. 1, 2005.0030 (2005); the GM08330 male iPS cell line (also known as GM8330-8) was provided by M. Talkowski (MGH) and was originally purchased from Coriell Institute. The H1 parental male hESC line (also known as WA01) was purchased from WiCell; and the 11a male iPS cell line was derived from the Harvard Stem Cell Institute.

All cell lines were authenticated. More particularly, the HUES66 hESC line were authenticated with STR analysis by GlobalStem in 2008 and the PGP1 iPS cell line with TRIPath 2018. The Mito210 iPSC line was authenticated with genotyping analysis (Fluidigm FPV5 chip) performed by the Broad Institute Genomics Platform (in 2017). The H1 and GM08330 was authenticated with STR analysis by WiCell in 2021. As previously described, the GM08330 parental line has an interstitial duplication in the long (q) arm of chromosome 20. All experiments involving human cells were performed according to ISSCR 2021 guidelines and approved by the Harvard University IRB and ESCRO committees.

### Cortical Organoid Differentiation

Dorsally patterned forebrain organoids were generated as previously described, with some adaptions to the protocol ((Velasco et al., Nature June 570 (7762): 523-527 (2019); Paulsen, Velasco, Kedaigle and Pigoni et al., Nature February 602 (7896): 268-273 (2022)). At day 0, feeder-free cultured human iPSCs or hESCs, 75-85% confluent, were enzymatically dissociated to single cells with Accutase (Gibco), and 9,000 cells per well were reaggregated in ultra-low cell-adhesion 96-well plates with V-bottomed conical wells (sBio PrimeSurface plate; Sumitomo Bakelite) in the same pluripotent medium in which they were previously maintained. At day 1, 80 μl of media is replaced with Cortical Differentiation Medium (CDM) I, containing Glasgow-MEM (Gibco), 20% Knockout Serum Replacement (Gibco), 0.1 mM Minimum Essential Medium non-essential amino acids (MEM-NEAA) (Gibco), 1 mM pyruvate (Gibco), 0.1 mM 2-mercaptoethanol (Gibco), with 1% of Penicillin-Streptomycin Solution (Corning). From day 0 to day 6, ROCK inhibitor Y-27632 (iROCK, Millipore) was added to the medium at a final concentration of 20 μM. Patterning small molecules, Wnt antagonist IWR-1-endo (Calbiochem) and TGFß inhibitor SB431542 (Stem Cell Technologies), were added from day 0 to day 15-18, at a concentration of 3 μM and 5 μM, respectively.

From day 15-18, patterned embryoid bodies (EB) were cultured under orbital agitation (Thermofisher CO2 resistant orbital shaker) in ultra-low attachment culture dishes (Corning) in CDM II medium, containing DMEM/F12 medium (Gibco), 2 mM Glutamax (Gibco), 1% N2 (Gibco), 1% Chemically Defined Lipid Concentrate (Gibco), 0.25 μg/mL fungizone (Gibco), with 1% of Penicillin-Streptomycin Solution (Corning). On day 35, cell aggregates were transferred to spinner-flask bioreactors (Corning) and maintained in CDM III (CDM II supplemented with 10% fetal bovine serum (FBS) (GE-Healthcare), 5 μg/mL heparin (Sigma) and 1% Matrigel (Corning)). From day 70, organoids were either cultured in CDM IV (CDM III supplemented with B27 supplement (Gibco) and 2% Matrigel) or Brainphys (StemCell Technologies) supplemented with 20 ng/mL GDNF (StemCell Technologies), 20 ng/mL BDNF (PeproTech), 500 μg/mL Dibutyryl-cAMP (StemCell Technologies), 1 μg/mL Laminin (Life Technologies), 200 nM Ascorbic Acid (StemCell Technologies), 1% N2 (Gibco), 1% B27 (Gibco), 2 mM Glutamax (Gibco), 0.1 mM MEM-NEAA (Gibco), 0.25 μg/mL fungizone (Gibco), with 1% of Penicillin-Streptomycin Solution (Corning).

### Multi-Genetic Background Cortical Chimeroid Generation

Cortical chimeroid generation is based in a re-aggregation process of “NSC” or “NPC” originating from dorsally patterned forebrain organoids at distinct developmental stages. For instance, NSC-Chimeroids and NPC-Chimeroids are derived from div15-18 and div23-25 patterned dorsal forebrain organoids, respectively.

At the desired developmental stage, brain cortical organoids were enzymatically dissociated into a single-cell suspension using the Worthington Papain Dissociation System kit (Worthington Biochemical). This protocol was specifically adapted to generate chimeroids (Quadrato et al., 2017b; Velasco et al., 2019b). Brain cortical organoids were carefully checked under the microscope to assess for proper morphology and collected into low-binding 1.5 mL tubes (Eppendorf). Carefully, the remaining media was discarded before adding the Papain solution. Previously warmed Papain solution (500 μl) was added to the tube, and organoids were minced (×10 times) using the P1000 pipette and incubated 25 min at 37° C. while shaking. Minced pieces were then mixed and gently broken up with a P1000 (×15), followed by an additional incubation of 5 min at 37° C. The resulting solution was moved to 15 mL tubes (Falcon) already containing 1 mL of Earle's balanced salt solution and 600 μl of inhibitor solution, then centrifuged 5 min at 300 g. The supernatant of the resulting solution was discarded, and dissociated cells were resuspended in 1 mL of CDM1 containing 20 μM of iROCK. To avoid aggregates and to generate a pure single-cell solution, dissociated cells were filtered using a 35 μm cell strainer tube (Corning) and counted with Countess II (Thermo Fisher Scientific). 18K-20K cells (depending on the number of donors) per well were reaggregated in ultra-low cell-adhesion 96-well plates with V-bottomed conical wells (sBio PrimeSurface plate; Sumitomo Bakelite) in CDM1 containing 20 μM of iROCK (10 mL of CDM1 per 96-well plate). The following day 85 μl of media was replaced with 100 μl of CDM2 per well. Two days after mixing, recently reaggregated EB's were passaged in 10 cm ultra-low attachment culture dishes (Corning) and cultured under orbital agitation (Thermofisher CO₂ resistant orbital shaker). The subsequent media changes of chimeroids follows the Velasco protocol, changing to CDM3 media at div35 and CDM4 or Brainphys media at div70.

### Telencephalic Chimeroid Generation

Telencefhalic chimeroid generation is based in a re-aggregation process as well (described in the previous section: Multi-genetic background, cortical chimeroid generation). The stability of differentially patterned “NPC” generation derived from subpallial organoids (adapted from Sartore et al.) and pallial or dorsal organoids (based on Velasco et al.) at div27 was used to advantage in this protocol.

At the desired developmental stage, the same dissociation process described above in the multi-genetic background chimeroid section was followed. To maintain a good pallial/subpallial ratio in the telencephalic organoids, the reaggregation step was followed with 85% ventrally derived-NPS and 15% dorsally derived-NPS. Finally, a total of 50K cells of both origins mixed were reaggregated per well in ultra-low cell-adhesion 96-well plates with V-bottomed conical wells in CDM1 containing 20 μM of iROCK. The following day 85 μl of media was replaced with 100 μl of CDM2 per well. Two days after mixing, recently reaggregated EB's were passaged in 10 cm ultra-low attachment culture dishes (Corning) and cultured under orbital agitation (Thermofisher CO₂ resistant orbital shaker). At div35 Telencephalic organoids were passed to CDM3 media, and subsequently to Brainphys media at div 55 (adapted from Velasco et al.).

### Multi-Species Chimera Generation

The generation of inter-species chimeras in vitro is based on the ability to synchronize the organoid dissociation of both species after their specific patterning period (described in previous section: Multi-genetic background cortical chimeroid generation). The inventors specifically took advantage of the capability to pattern different species independently of each other's requirements; in this way, patterned factors, time of exposure and media specification can be applied in a species-specific manner without disruption of their innate features. The stability of species-specific differentially pattern “NSC” allowed the intermingling of a desired percentage of human NSCs (hNSCs) into a mouse-specific environment, depending on the objective of the study. For instance, one could potentially integrate a reduce percentage of mouse NSCs (mNSCs) in a human environment as well, highlighting the potential flexibility of the method.

At the species-specific developmental stage, div 15 and div 5 for human and mouse cortical organoids, respectively, the same dissociation process described above in the multi-genetic background chimeroids section was followed. To maintain a major mouse environment in the multi-species cortical chimeras, the reaggregation step was followed with 85% mouse derived-NSCs and 15% human derived-NSCs. Finally, a total of 15-18K cells of both origins mixed were reaggregated per well in ultra-low cell-adhesion 96-well plates with V-bottomed conical wells in CDM1 containing 20 μM of iROCK. The following day 85 μl of media was replaced with 100 μl of CDM2 per well. Two days after mixing, recently reaggregated EB's were passaged in 10 cm ultra-low attachment culture dishes (Corning) and cultured under orbital agitation (Thermofisher CO₂ resistant orbital shaker). Following mouse time-line, at div11 multi-species chimeras were passed to CDM3 media and subsequently to Brainphys media at div21 (adapted from Velasco et al. and unpublished data from Anton-Faravelli et al.).

### Potential of the Chimeroid Methodology

A new methodology is described herein to generate more complex brain organoids, from multi-genetic backgrounds in the context of human dorsal forebrain development to the possibility of studying species-specific features in human-mouse chimeric organoids. Moreover, the great plasticity of this system allows integration of cell types derived from different developmental origins, for instance pallial/subpallial origins, to form a more complex telencephalic entity. The chimeroid model can be used to generate more complex structures as well, adding cell types from very diverse origins like endothelial or immune cells to generate a better vascularized system, or photoreceptors that can act as electrical stimulators that potentially will have the capability to generate evoked electrical responses in the brain cortical cells. Thus, this powerful tool will enable the production of organoids where the proportions of each cell type can be controlled, and thus defined circuits can be studied.

Dissociation of Brain Organoids and scRNA-Seq

Organoids were dissociated as previously described, with an exception for one-month-old organoids that were dissociated using low-binding 1.5 mL tubes (Eppendorf) described in detail in the chimeroid generation section. Dissociated cells were resuspended in 100 μl of PBS and filtered with a 35 μm cell strainer tube (Corning) to avoid aggregates and counted with Countess II (Thermo Fisher Scientific). Single-cell suspension was loaded into the Chromium™ Next GEM Chip G (10× Genomics) and run with the Chromium Controller to generate single cell GEMs. scRNA-seq libraries were prepared with the Chromium™ Single Cell 3′ Library and Gel Bead Kit v3 and v3.1 (10× Genomics). As chimeroids are formed by multiple genetic backgrounds, they were treated as upstream multiplexed samples; therefore the Chromium™ Next GEM Chip was loaded with ~30K cells per Chip. Libraries were pooled from different samples based on molar concentrations and sequenced on a NextSeq 500 or NovaSeq 6000 instrument (Illumina) with 28 bases for read 1 (26 bases for v2 libraries), 55 bases for read 2 (57 bases for v2 libraries), and 8 bases for Index 1. If necessary, after the first round of sequencing, libraries were re-pooled based on the actual number of cells in each and re-sequenced with the goal of producing an equal number of reads per cell for each sample.

### Cryopreservation

Chimeroids were processed as previously described (Faravelli-Rinchetti et al., unpublished data). Samples were fixed in 4% paraformaldehyde (PFA) (Electron Microscopy Services) overnight (O/N) in a 12 well plate (falcon) at 4° C., washed with 1× phosphate buffered saline (PBS, Gibco)×3 times and cryoprotected in a 30% sucrose solution in PBS (O/N) at 4° C.

### Inclusion in Bovine Gelatin

Gelatin solution containing 10% of bovine gelatin (Sigma) and 7.5% of sucrose (Sigma) was prewarmed at 37° C. for 15 min. 30% sucrose solution was exchanged for the prewarmed gelatin and incubated at 37° C. for 15 min. Meanwhile, plastic molds were coated with a 2 mm layer of warm gelatin solution and left to polymerize at room temperature (RT). Chimeroids were transferred to the plastic mold (previously coated), and 1 mL of warm gelatin solution was added to completely cover the chimeroids. Following polymerization at RT for 3 min, they were moved into 4° C. for 15-20 min. Finally, the molds were frozen in a cold bath containing 100% EtOH and dry ice for 2-3 min and stored at −80° C. indefinitely.

### Immunohistochemistry

For immunohistochemistry, 14 to 20 μm thick sections were cut using a cryostat (Leica). Cryosections were stabilized at RT for 5 min and blocked with 10% donkey serum (Sigma)+0.3% Triton X-100 (Sigma) in PBS. Primary antibodies were diluted in the blocking solution and incubated O/N. After 4 washes with PBS, cryosections were incubated at RT with secondary antibodies diluted in PBS (1:1000) for 1 hour at room temperature, washed 4× with PBS followed by DAPI staining (1:10,000 in PBS+0.1% Tween-20) for 15 minutes to visualize cell nuclei. The protocol was adapted to 1 day for specific antibodies (table X). In this case, primary antibodies were incubated for 3 h at RT. In case antigen retrieval was needed to unmask desired antigens; prior blocking solution, cryosections were place in a coplin jar with 1× Antigen retrieval citrate buffer pH 6.0 (IHC select-Millipore) bring to boil in a microwave for 30-45 sec, let it cold down for 40 min and wash 3 times with 1×PBS.

### Microscopy

Immunofluorescence images were acquired with a Zeiss Axio Imager.Z2 and Lionheart™ FX Automated Microscope (BioTek Instruments). Images were analyzed and processed with the Gen5 (BioTek Instruments) and Zen Blue (Zeiss) image processing software, and ImageJ (Fiji).

### Example 2—Multi-Donor Human Cortical Chimeroids Reveal Individual Susceptibility to Neurotoxic Triggers

Genetic variation plays a crucial role in differential susceptibility to disease triggers (Paulsen et al., Nature, 2022; Pizzo et al., Genet Med, 2019), such that different individuals show heterogeneous responses to disease risk factors. However, while the effects of human genetic variation can be detected in in vitro systems (Ford et al., Toxics, 2022; Germain and Testa, Stem Cell Reports, 2017; Paulsen et al., Nature, 2022;

Tegtmeyer et al., bioRxiv, 2023), exploring the mechanisms that mediate the effect of common and rare genetic variants on physiological phenotypes and that drive differential responses between individuals is challenging. Investigation of these mechanisms is hampered by the limited availability of human experimental models that are both faithful to human endogenous biology and can be scaled across many individuals.

In the brain, pioneering studies have adapted pluripotent stem cell (PSC)-derived 2-dimensional (2D) neuronal cultures to produce population-scale models comprising multiple donor cell lines of different genetic backgrounds (Cederquist et al., Cell Stem Cell, 2020; Cuomo et al., Nat Commun, 2020; Jerber et al., Nat Genet, 2021; Limone et al., Cell Rep, 2023; Mitchell et al., bioRxiv, 2020; Wells et al., Cell Stem Cell, 2023). However, 2D cultures typically produce a limited spectrum of cell types that does not reflect the diversity of the endogenous brain. Three-dimensional (3D) cell culture models such as human brain organoids can more closely model the cellular complexity and developmental events of the endogenous brain, and thus may be used to test the effects of inter-individual variation across a wider range of cell types and developmental processes. However, efforts to extend the 2D “village-in-a-dish” approach (Mitchell et al., bioRxiv, 2020; Wells et al., Cell Stem Cell, 2023) to 3D organoid cultures have been limited because differences in growth rate and differentiation biases between PSC lines lead to unbalanced donor representation in multiplexed PSC differentiation systems, such that certain lines take over the cultures or are eliminated. Prior selection of PSC lines with matched growth rates can ameliorate the problem, (Wells et al., bioRxiv, 2021), but limits experimental design, in that many PSC lines cannot be used. Such biases are particularly problematic for organoid systems, where multiple cell types co-develop over long time periods, exacerbating the effect of growth or differentiation biases (Jerber et al., Nat Genet, 2021).

Here, this challenge was addressed by developing 3D multi-donor Chimeroids: a highly-reproducible, human cortical organoid model that preserves balanced representation of cell types derived from panels of donors, without the need for PSC pre-selection. By fine-tuning the developmental stage when donor line multiplexing occurs, the inventors diminished differences in growth biases among donors in the resulting chimeroids. This model was used to measure inter-individual susceptibility to developmental exposure to two neurotoxic stressors was developed: ethanol (EtOH, associated with Fetal Alcoholic Spectrum Disorder (FASD) (Wozniak et al., Lancet Neurol, 2019)) and the anti-epileptic drug valproic acid (VPA, associated with increased risk of Autism Spectrum Disorders (ASD) (Bjørk et al., JAMA Neurol, 2022; Christensen et al., JAMA, 2013)). EtOH and VPA exposure resulted in changes across multiple cell types within Chimeroids, and, importantly, Chimeroids captured donor-specific differences in response. Overall, the findings show that inter-individual variation is a significant source of susceptibility to neurotoxic triggers, and that Chimeroids can be used as a scalable platform to measure variation in biological response in brain tissue of different human individuals.

### Results

### Brain Chimeroids Produce Cortical Cell Diversity Balanced Across Individual Donors

To develop a human brain system that can model variation in responses by different individuals to disease risk factors, such as genetic variants or neurotoxic insults, a strategy for generating multi-donor human brain organoids was devised, in which each cell type would be produced from multiple donor lines within a single brain organoid.

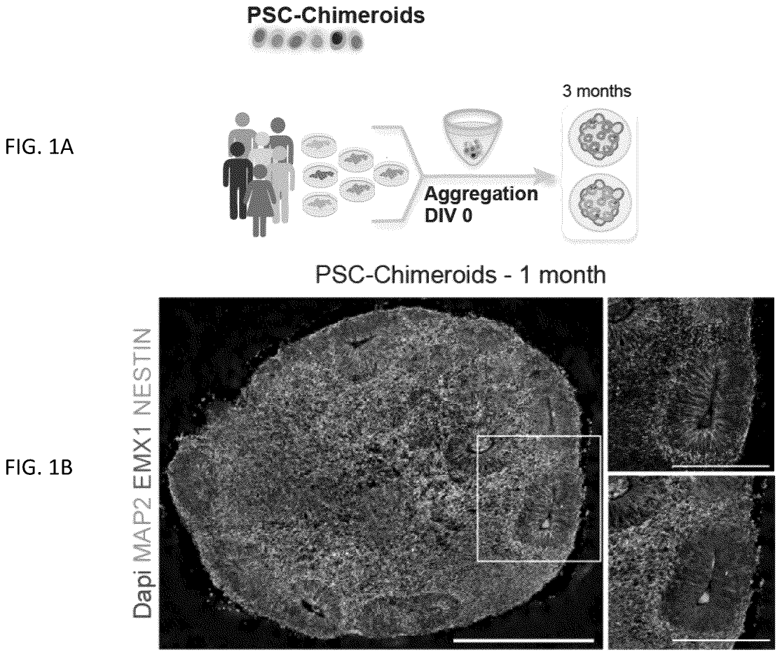

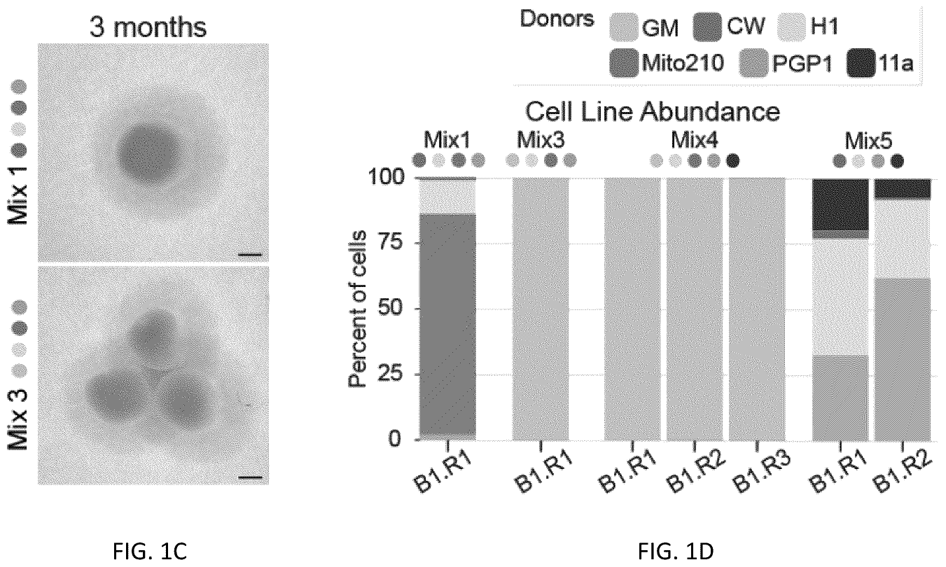

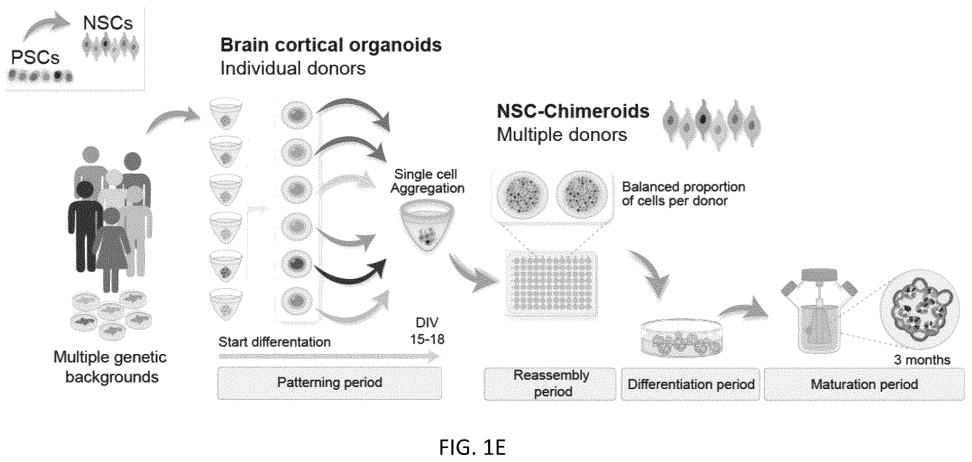

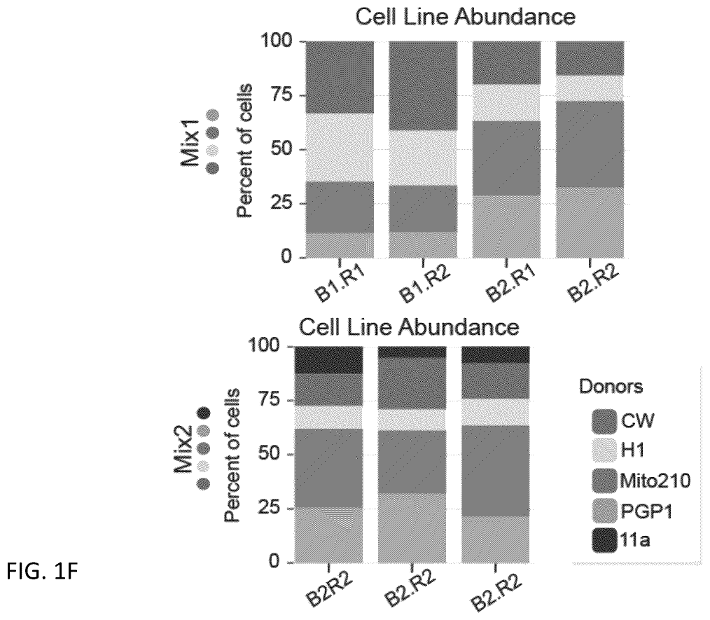

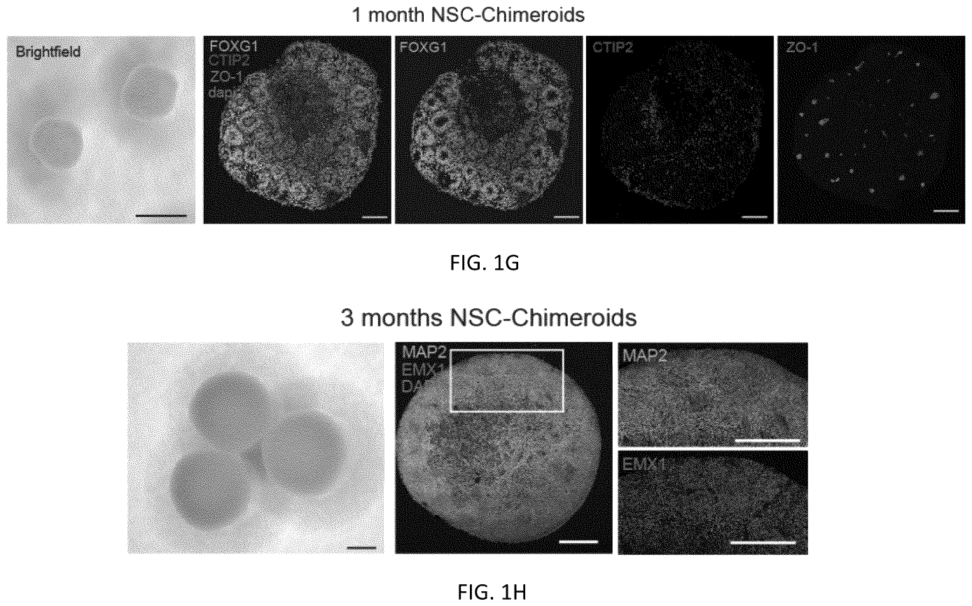

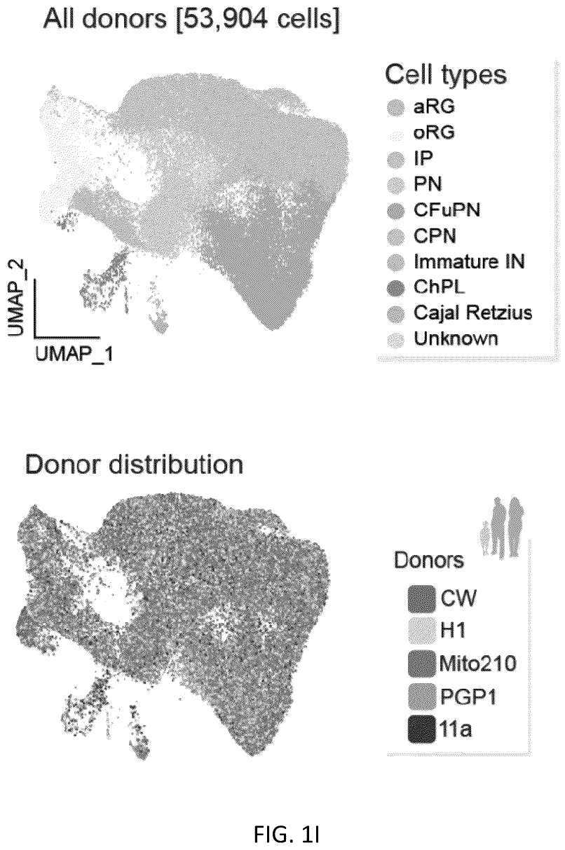

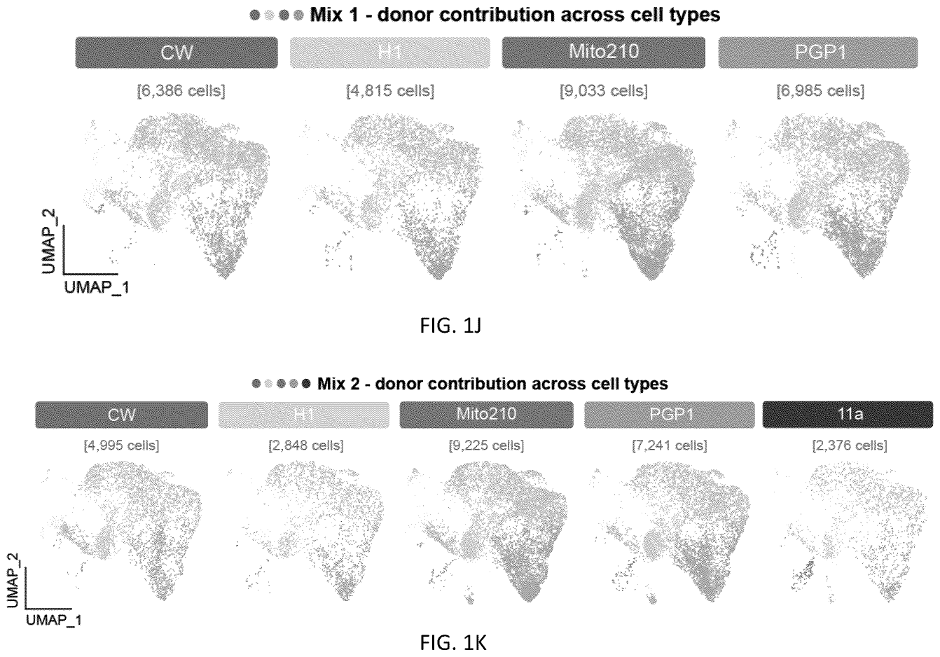

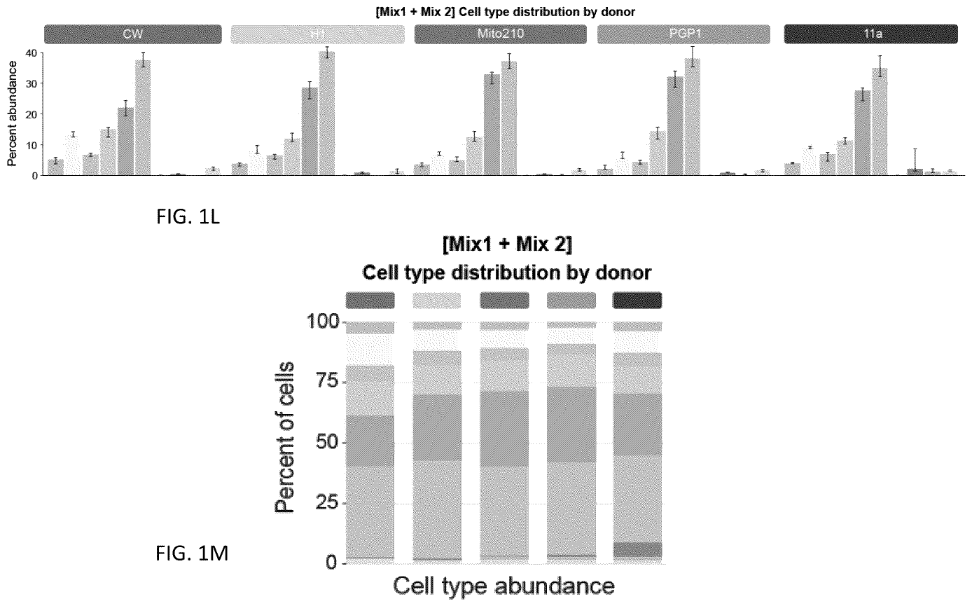

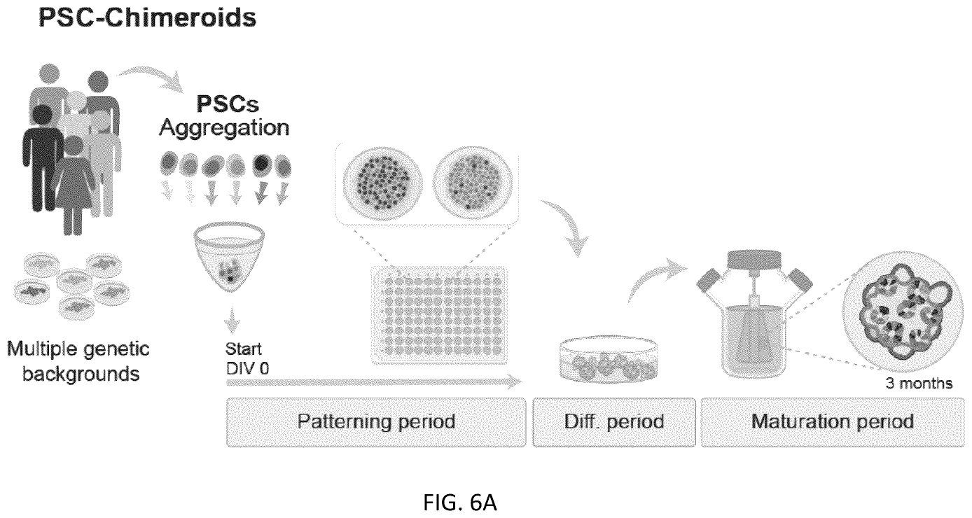

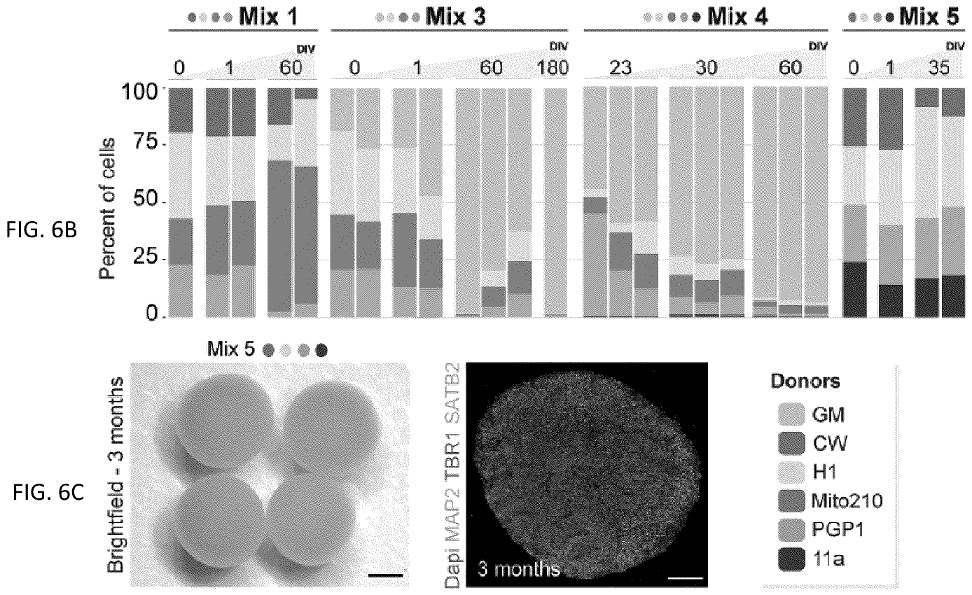

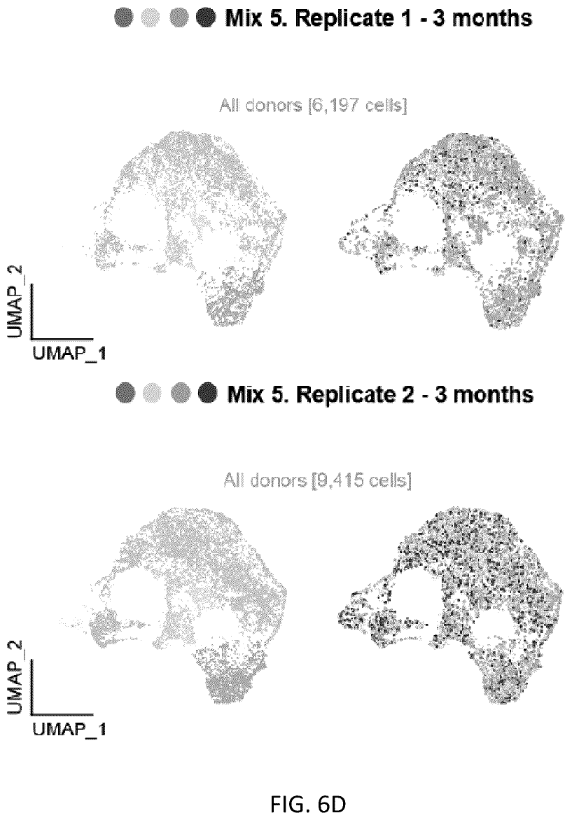

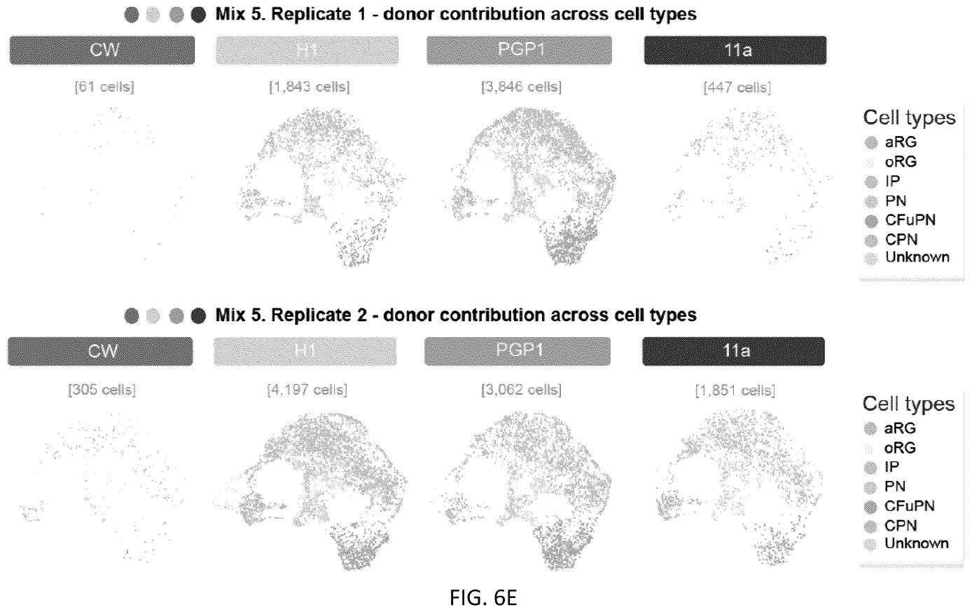

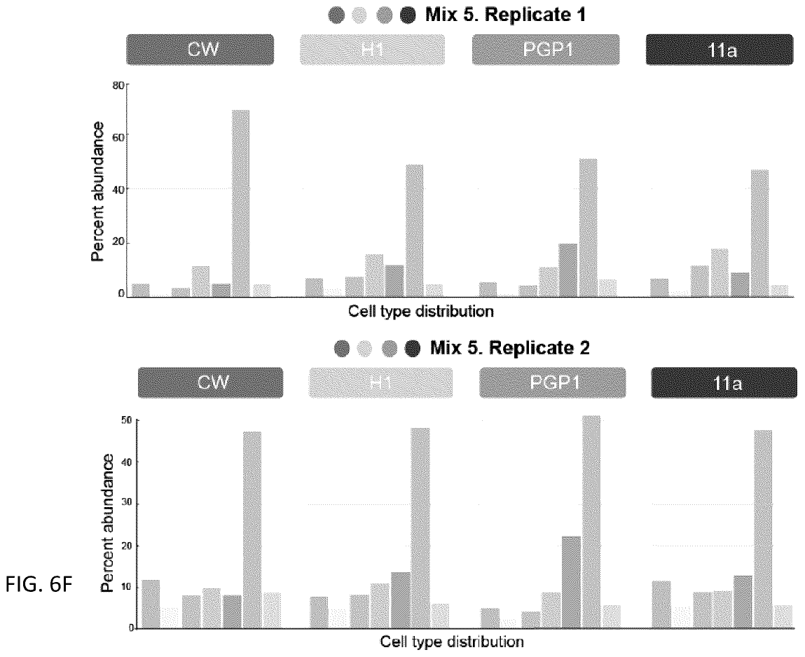

Because human PSCs from multiple donors can be grown and differentiated together in 2D cultures (Cederquist et al., Cell Stem Cell, 2020; Cuomo et al., Nat Commun, 2020; Jerber et al., Nat Genet, 2021; Limone et al., Cell Rep, 2023; Mitchell et al., bioRxiv, 2020; Neavin et al., bioRxiv, 2021; Villa et al., Cell Rep, 2022; Warren and Cowan, Trends Cell Biol, 2018; Wells et al., Cell Stem Cell, 2023), the inventors first attempted to generate Chimeroids by mixing 4 to 5 lines in equal ratios at the PSC stage (referred to here as PSC-Chimeroids; FIGS. 1A-1C and FIGS. 6A-6F). The inventors used the following human lines: H1 (male, embryonic stem cell [ESC]), Mito210 (male, induced pluripotent stem cell [iPSC], control with no familial history of neurological disorders), PGP1 (male, iPSC, control), CW50037 (hereafter ‘CW’; female, iPSC, control with no familial history of neurological disorders), GM08330 (hereafter ‘GM’; male, iPSCs, clinically-unaffected with familial history of relevant psychiatric illness), and 11a (male, iPSC, control).

Mixes of PSC lines were aggregated in 96-well plates to form embryoid bodies (EBs); then, following the previously-published protocol (Velasco et al., Nature, 2019), EBs were patterned for 15-18 days to induce dorsal forebrain fates, and nascent organoids were then switched to CDMII media and placed in dynamic (agitated) culture to allow maturation (Methods). The inventors quantitatively determined the contribution of individual donor lines to each organoid at different stages of development using Census-seq (Mitchell et al., bioRxiv, 2020), which uses low-coverage whole genome sequencing (WGS) to determine donor proportion.

At 1 month in vitro (1 mo), PSC-Chimeroids showed unbalanced contribution of the individual donors, which became more pronounced upon further culture, at 3 mo (FIG. 1D and FIG. 6B). The inventors tested four different combinations of donors, from distinct experiments, at different time points, and observed unbalanced growth in all cases (FIG. 1D and FIG. 6B). Specifically, the inventors analyzed: Mix 1 (4 lines: CW, H1, Mito210, PGP1), Mix 3 (4 lines: GM, H1, Mito210, PGP1), Mix 4 (5 lines: GM, H1, Mito210, PGP1, 11a) and Mix 5 (4 lines: CW, H1, PGP1, 11a). PSC-Chimeroids that included GM (Mix 3 and Mix 4) had pronounced overgrowth of this line, which almost completely took over the Chimeroids by 2 mo (FIG. 6B). At 23 days in vitro (DIV23), 54% of cells in Mix4 PSC-Chimeroids were GM-derived (FIG. 6B), and single-cell RNA-seq (scRNA-seq) on Chimeroids from this mix followed by supervised donor assignment by genetic variants (Kang et al., Nat Biotechnol, 2018) (Methods) showed that at 3 mo the GM line contributed almost all (>99%) of the cells in the PSC-Chimeroids (FIG. 1D). In the other mixes (without GM), the Mito210 cell line was disproportionately overrepresented (84% of the cells in Mix 1 PSC-Chimeroids at 3 mo, FIG. 1D). In Mix 5 PSC-Chimeroids, which had neither GM nor Mito210, one line (CW) was almost eliminated (<3% of the Chimeroid cells at 3 mo, FIG. 1D and FIG. 6D). The inventors conclude that mixing different PSC lines at the initiation of EB formation leads to vastly disproportionate representation of individual donors, a finding that is consistent with previous observations in a dopaminergic neuron model (Jerber et al., Nat Genet, 2021) Despite these differences between lines, when multiple donors maintained significant representation (Mix 5), all retained donors were able to contribute to all identifiable cell types in a largely balanced manner, indicating that the Chimeroid approach does not per se alter cell fate potential in individual donors (FIG. 1B and FIGS. 6D-6F).

PSCs are highly proliferative and plastic in fate potential, and small differences in these properties early on during organoid development may lead to disproportionate representation at later times. To constrain such inter-line biases, it was hypothesized that mixing different donor lines after PSCs have undergone neural fate commitment, when they have reduced cell growth and more restricted fate potential, would reduce variability in terminal donor contribution. The inventors therefore modified the Chimeroid protocol such that donor mixing would occur after completion of neural patterning, at the neural stem cell (NSC) stage (referred to hereafter as NSC-Chimeroids) (FIG. 1E).

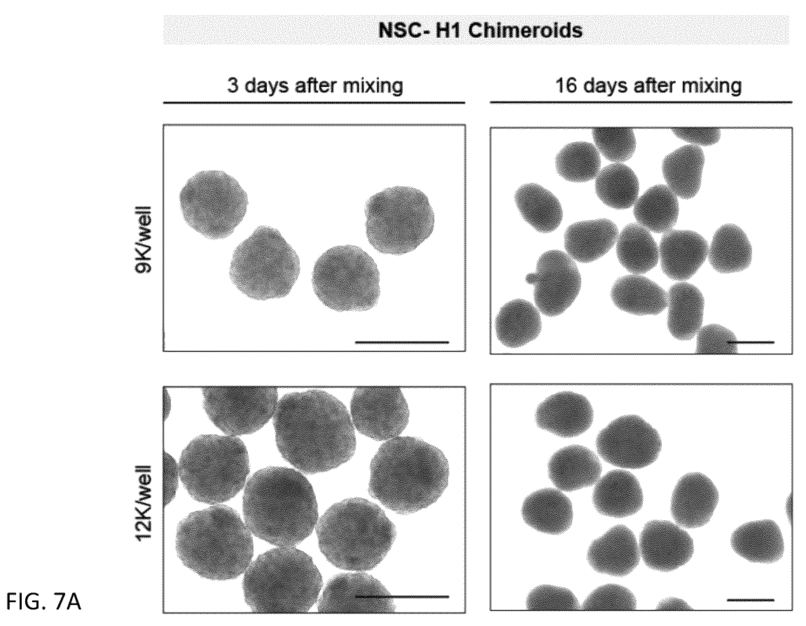

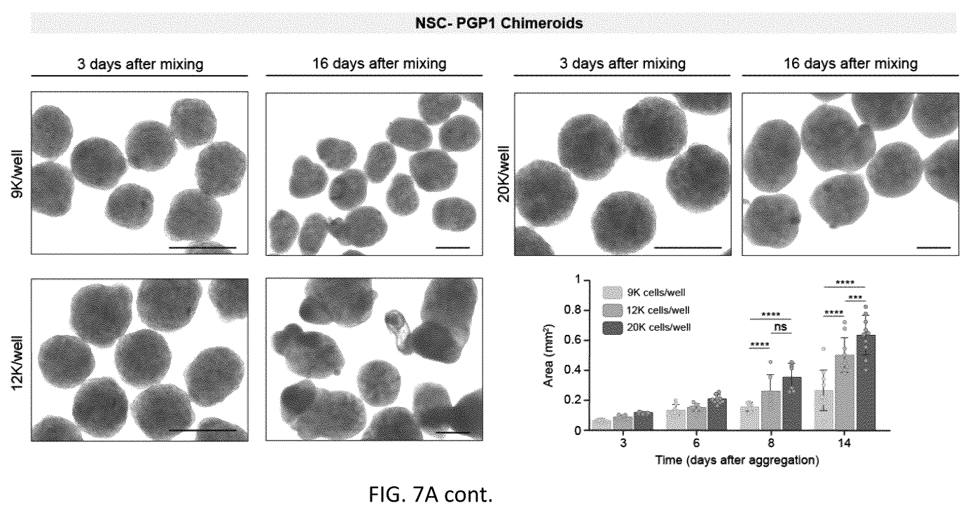

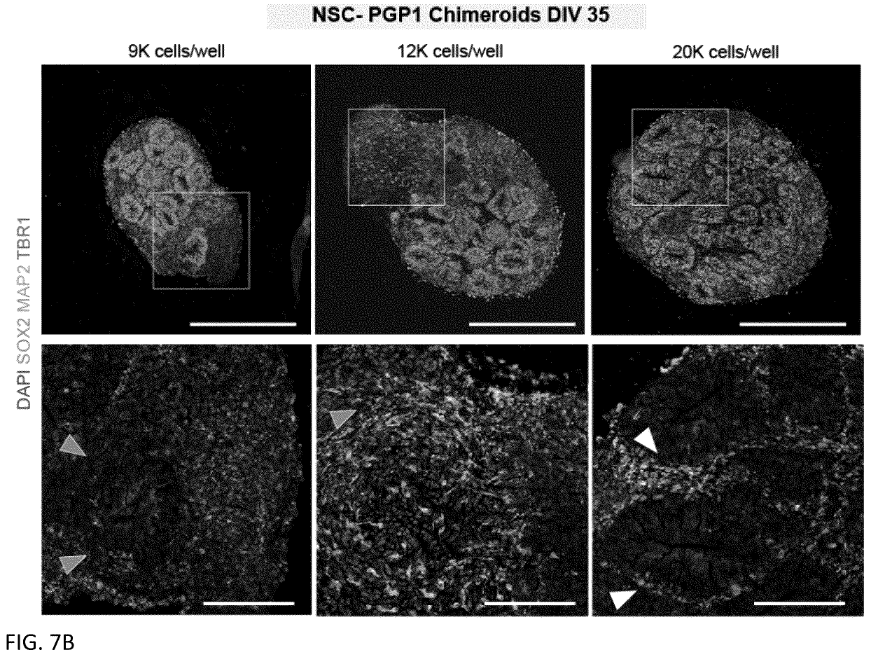

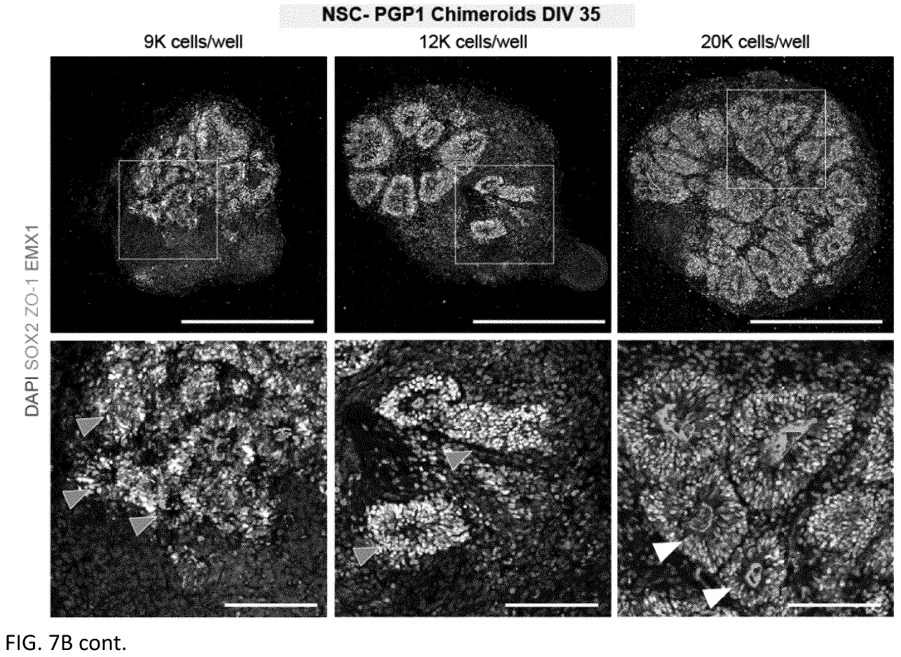

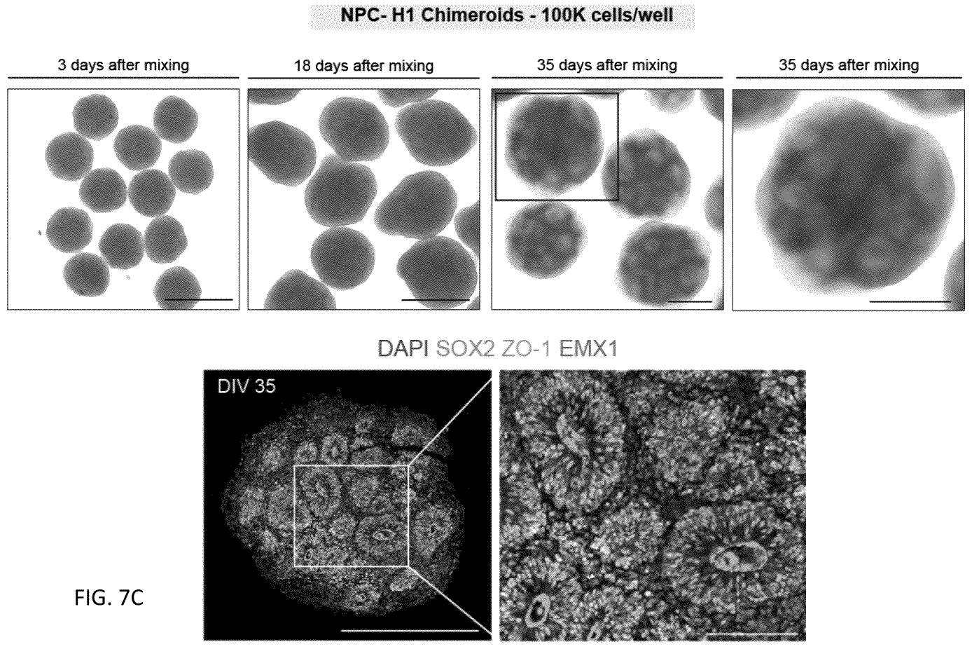

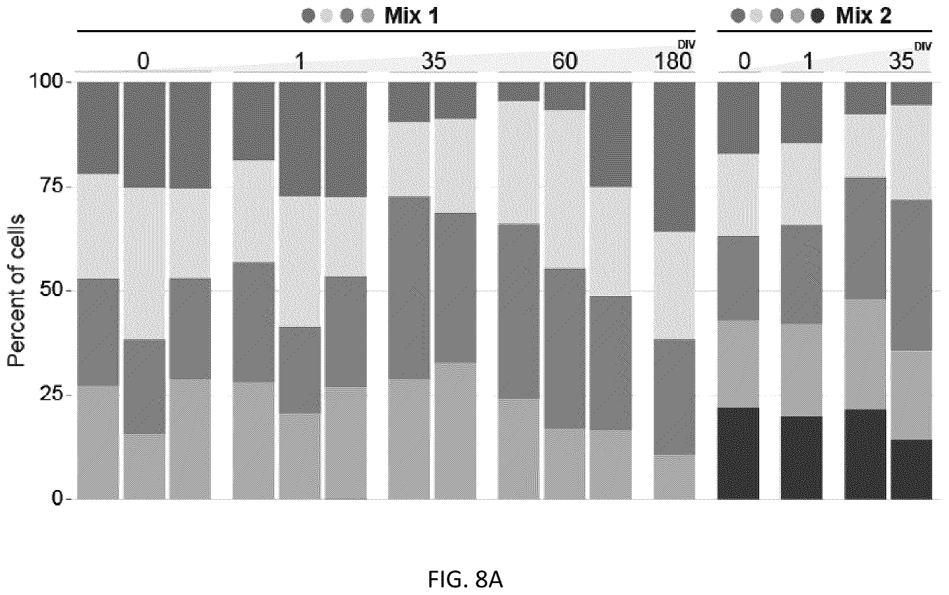

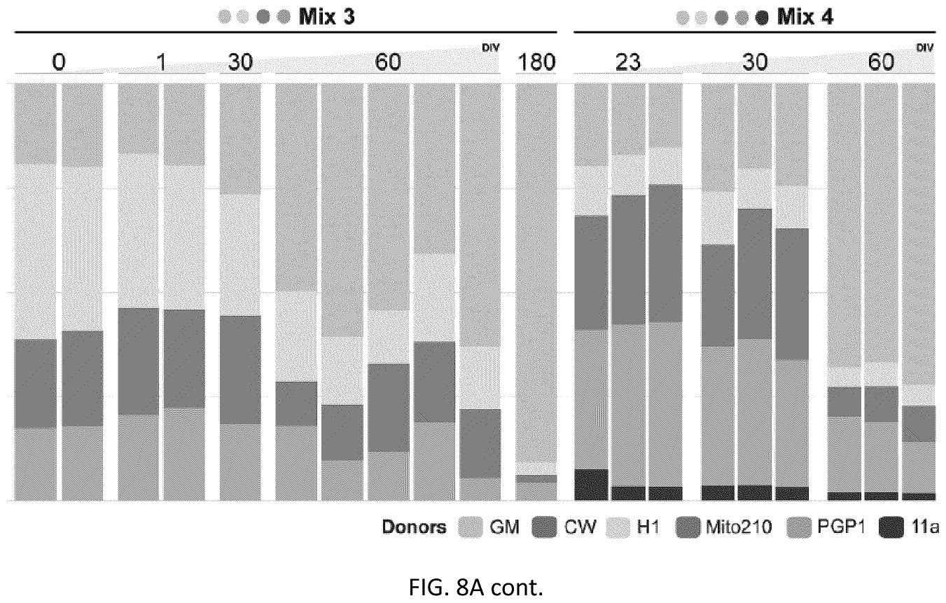

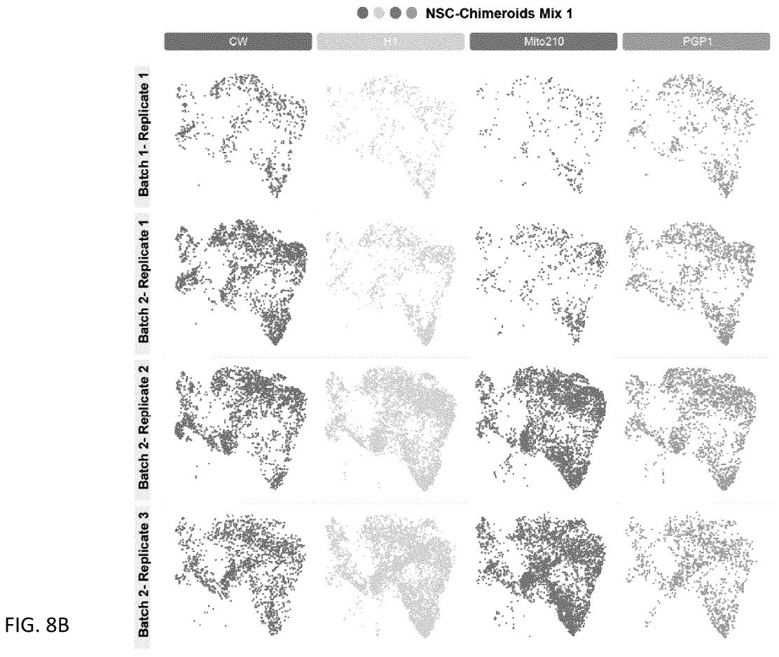

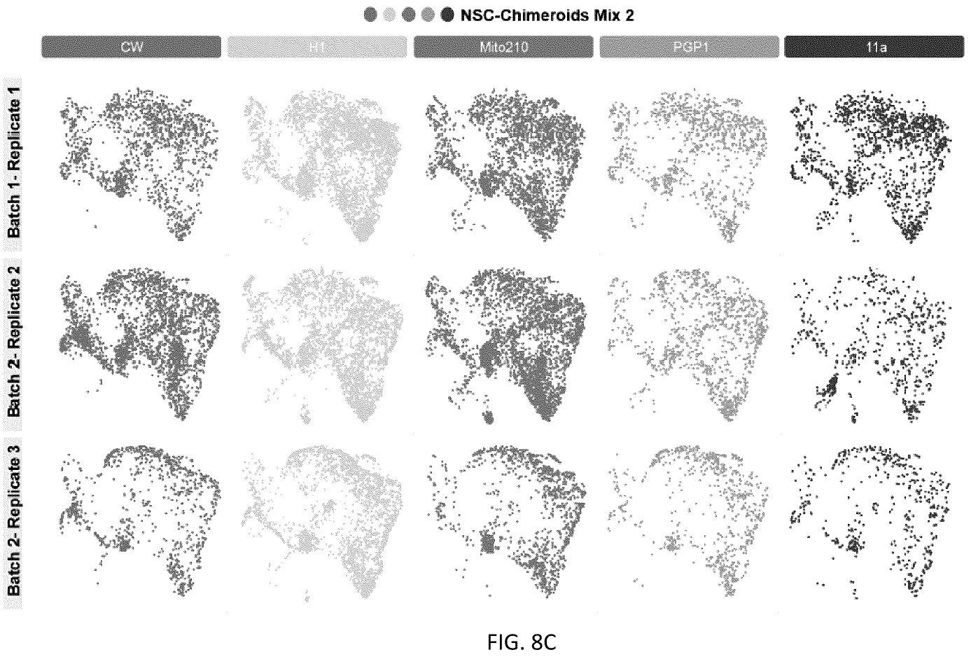

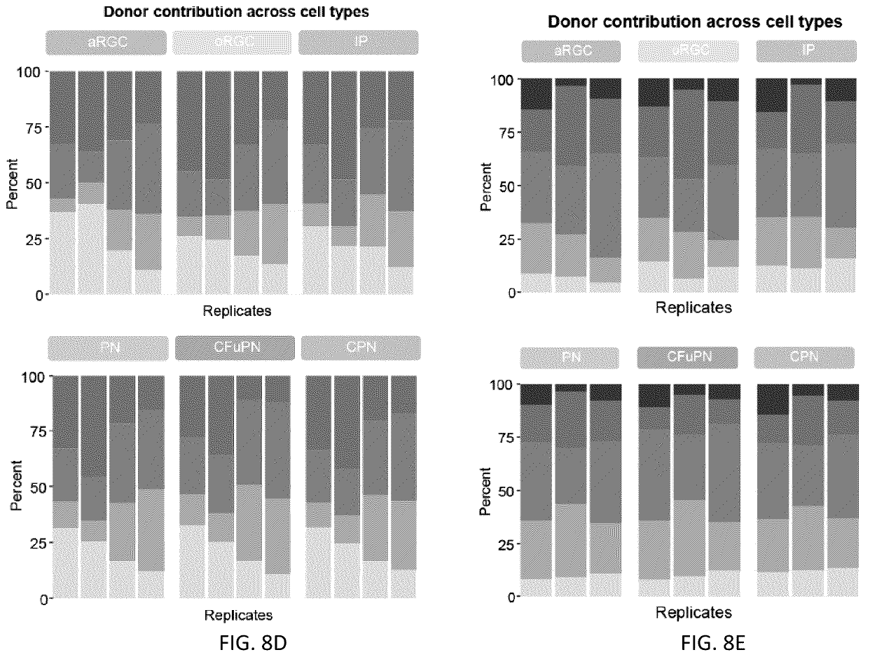

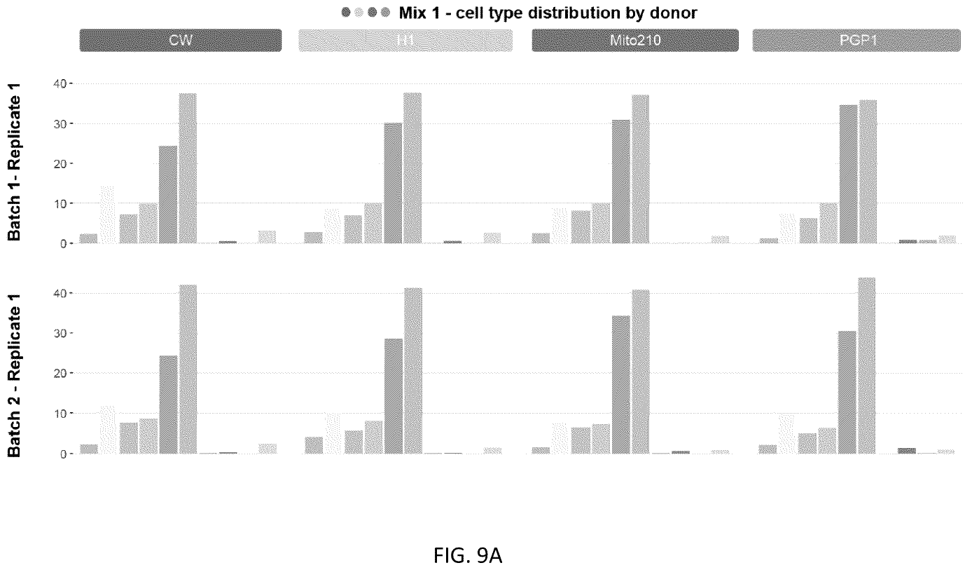

To create NSC-Chimeroids, the inventors first generated single-donor EBs, and patterned them with small molecules until DIV15-18 (Velasco et al., Nature, 2019). Nascent single-donor organoids were dissociated from 4 to 5 individual lines to single cells, and mixed donor cells at equal proportions (DIVO, FIG. 8A). The inventors then seeded the pooled cells in 96-well plates, at 20,000 cell/well (Methods, FIG. 7 for choice of this cell density) and allowed them to re-aggregate for 2 days before transfer to dynamic culture (FIG. 1E and Methods). At 20 days after aggregation, NSC-Chimeroids had developed ‘rosettes’, prospective ventricular zone-like (VZ-like) structures composed of cells expressing the telencephalic marker FOXG1, surrounded by nascent corticofugal projection neurons (CTIP2+); the rosette centers were lined with ZO-1, a tight junction protein present in the endfeet of apical radial glia (aRG), indicating correctly-polarized epithelium (FIG. 1G).

Quantification by Census-seq (n=36 individual Chimeroids) and scRNA-seq (n=7 individual Chimeroids, 53,904 cells) showed that NSC-Chimeroids maintained substantially more balanced contribution of each donor line, in each of 4 different mixes (FIG. 1F and FIG. 8A). Notably, Mix 1, 3 and 4, which in PSC-Chimeroids were dominated (85% to 99%) by a single donor, retained contribution of all donors in NSC-Chimeroids. Thus, mixing donor lines at the NSC stage improves retention of individual donors, compared to mixing at the PSC stage.

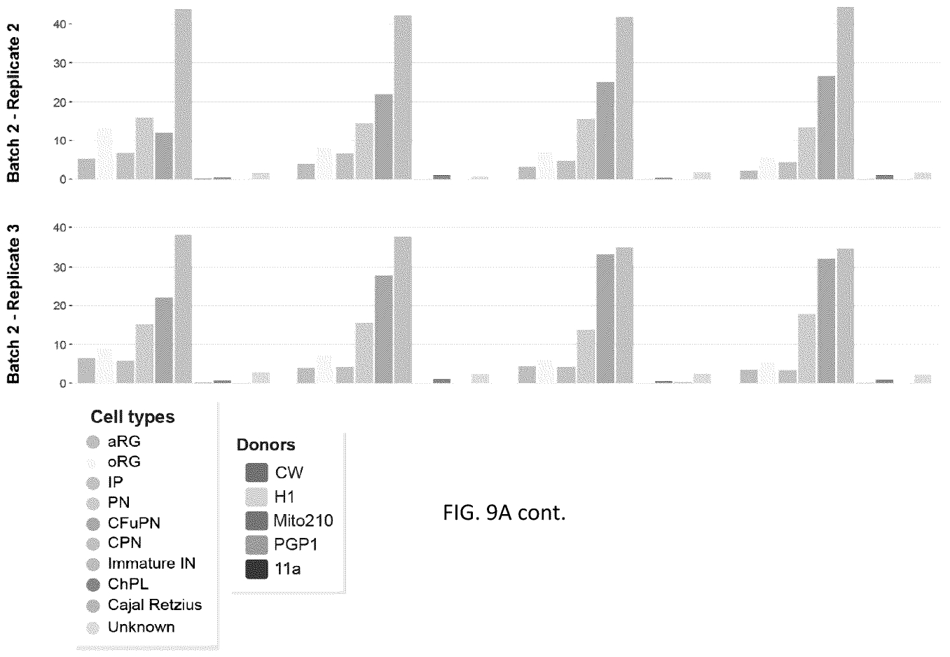

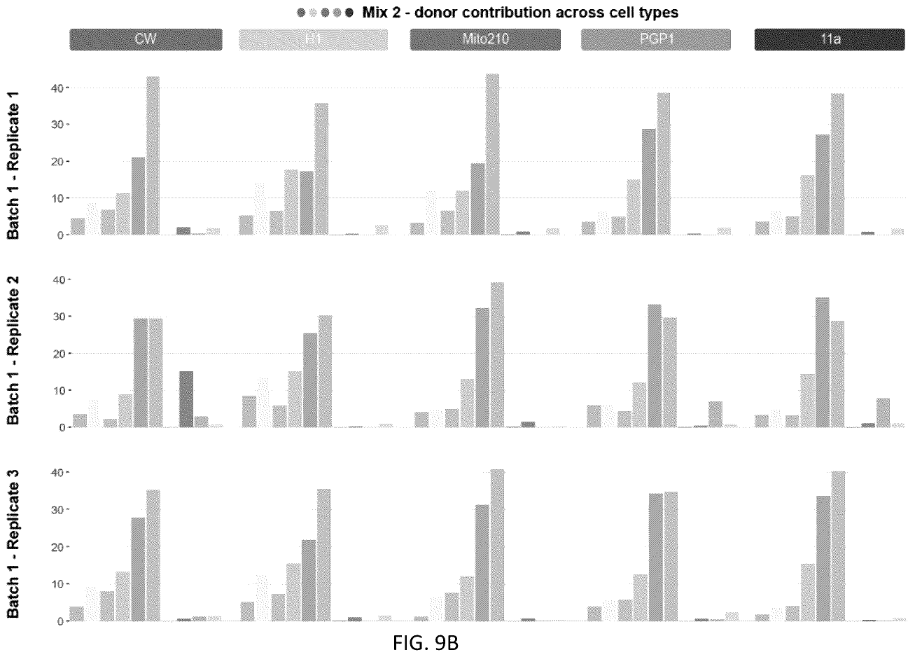

At 3 mo, NSC-Chimeroids showed proper expression of dorsal forebrain markers, at the protein level (FIG. 1H). ScRNA-seq of Mix 1 and Mix 2 NSC-Chimeroids at 3 mo (n=7 individual Chimeroids, 53,904 cells) showed that both mixes generated the full compendium of cell types expected from single-donor dorsal forebrain organoids produced by the original protocol (Velasco et al., Nature, 2019) (FIGS. 1I-1K). Importantly, each donor contributed to the generation of each cell type (FIGS. 1L-1M, FIGS. 8B-8E, FIG. 9).

### Chimeroids can Control Donor Biases in Proliferation

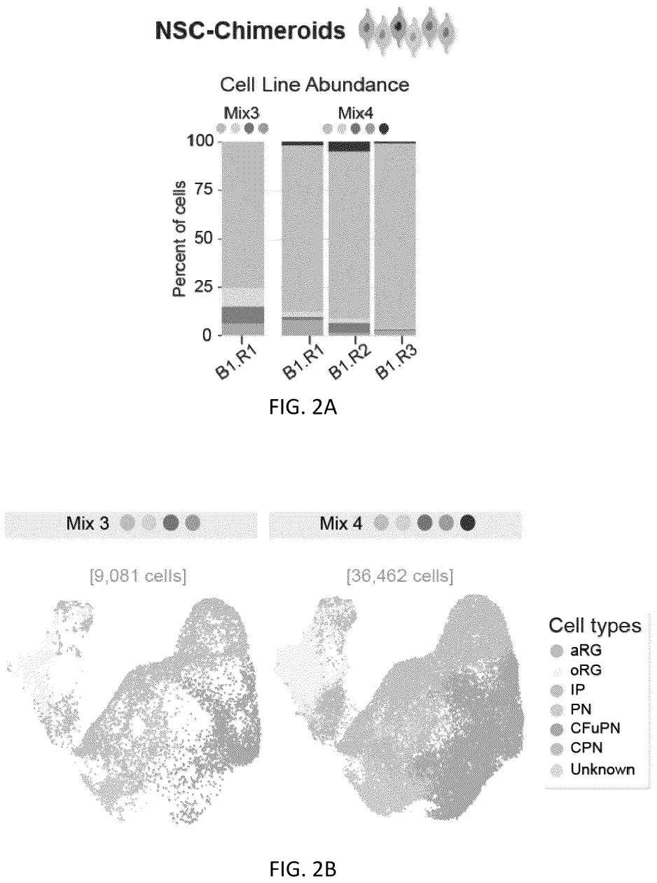

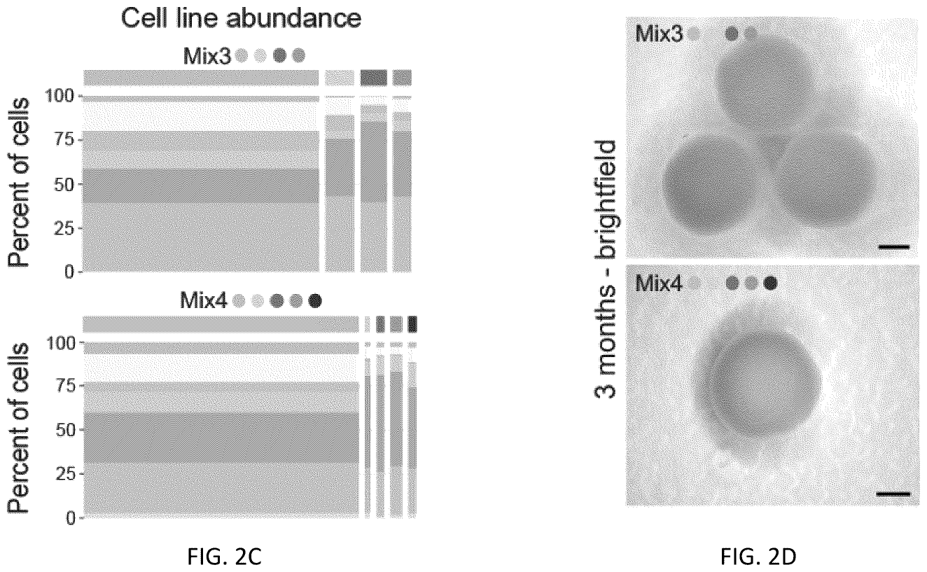

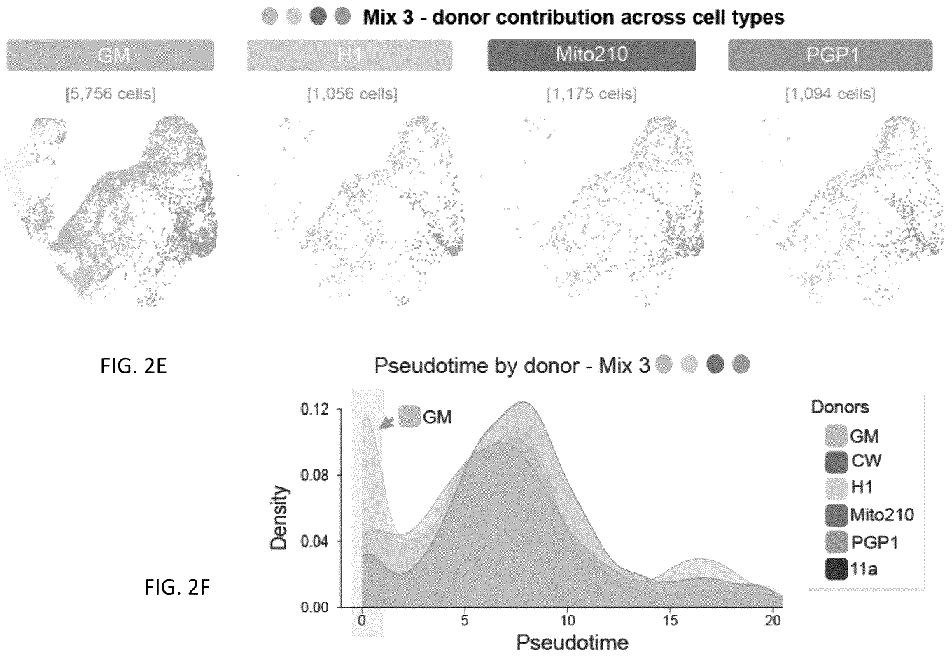

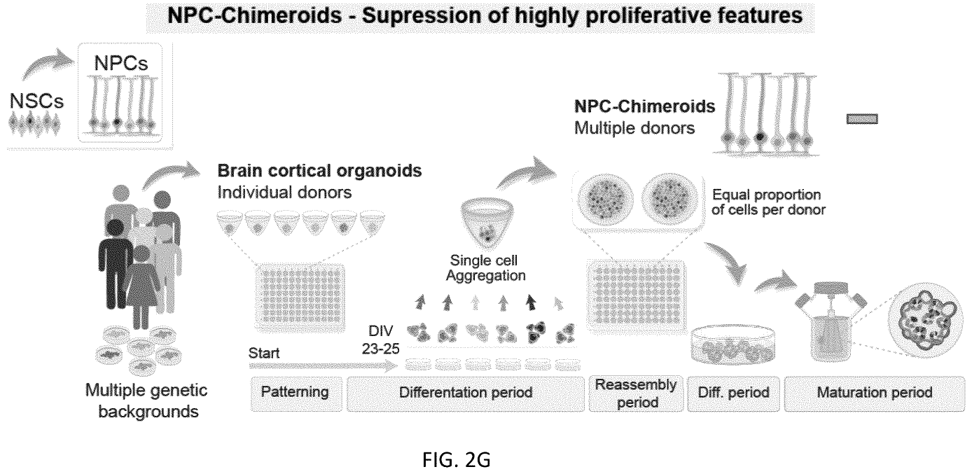

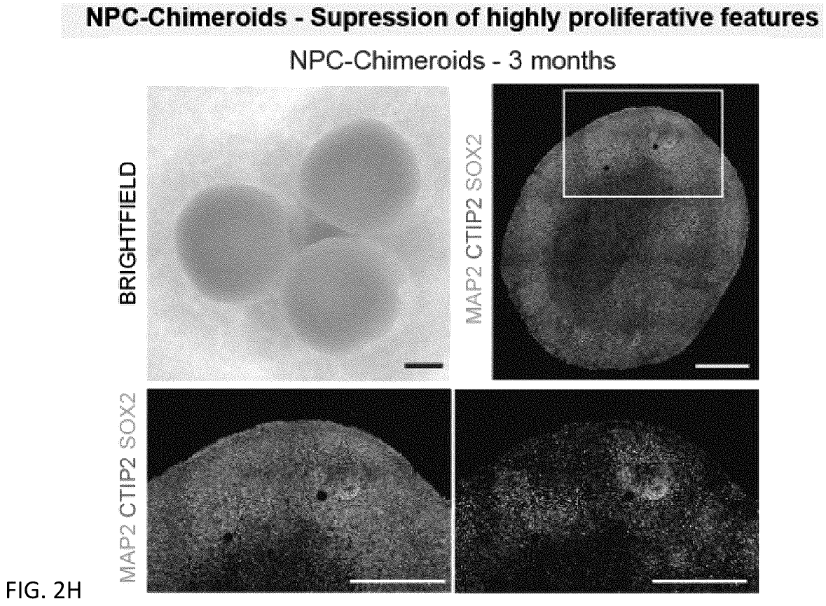

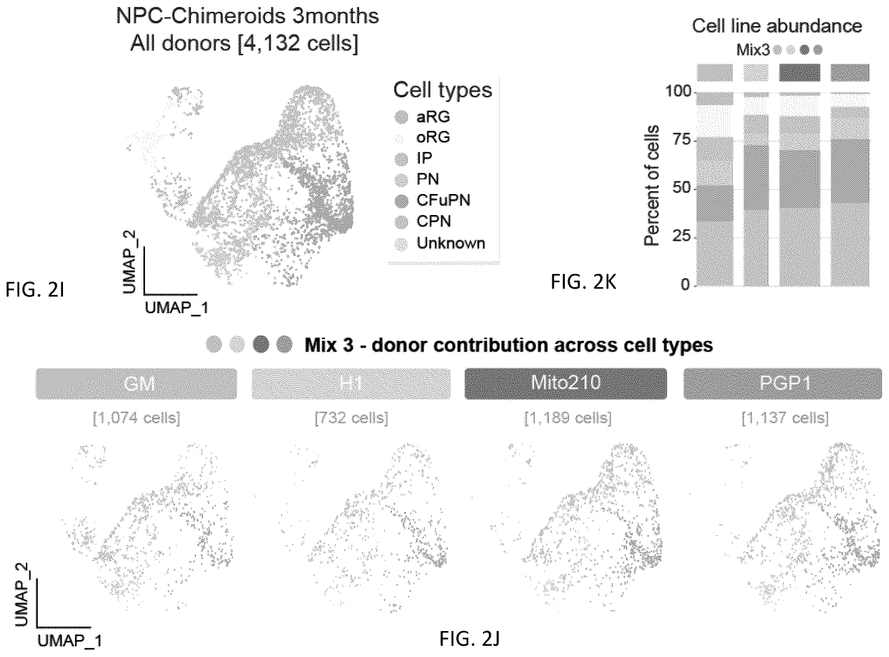

To investigate the potential of Chimeroids to buffer against donor variation in growth rates, the inventors challenged the system by including one iPSC line with a dramatic proliferative advantage (GM) (FIG. 1D). The inventors created multi-donor NSC-Chimeroids from two additional mixes of 4 or 5 lines: Mix 3 (GM, H1, Mito210, and PGP1) and Mix 4 (GM, H1, Mito210, PGP1, and 11a). Although incorporating GM skewed the proportions of donors in NSC-Chimeroids (GM contributing 76-95% of cells; FIGS. 2A-2C), at 3 months all donors were represented and able to generate all the cell types expected at this stage (Uzquiano et al., Cell, 2022) (FIGS. 2C-2E). GM produced a higher proportion of neural precursor cells (mean=31.0%, sd=3.28%) compared to other donors (mean=13.8%, sd=6.4%), showing a particular expansion of oRGs (GM: mean=16.1%, sd=0.6%; other donors: mean=6.8%, sd=2.6%; FIG. 2C), thus pointing at possible reasons for the growth advantage of the GM line. Consistent with this, pseudotime analysis suggested an increased proportion of cells predicted to occupy earlier developmental stages in GM-derived cells compared to cells from other donors (FIG. 2F).

Following this observation it was hypothesized that mixing progenitors at even later, less-proliferative stages of differentiation and fate commitment might further improve donor growth biases. The inventors therefore generated Chimeroids from neural progenitor cells (NPCs), by dissociating and mixing cells at a later stage of the organoid protocol, at DIV23-25 (Mix 3: GM, Mito210, H1, PGP1) and profiled these NPC-Chimeroids by scRNA-seq (1 batch, n=2, 4,132 cells) at DIV90 (FIGS. 2G-2K). NPC-Chimeroids achieved the expected cell type composition (FIG. 2I) and, importantly, had more balanced donor contribution, even in the presence of the GM line that overwhelmingly dominated the PSC-Chimeroids. Specifically, at DIV90, GM contributed 17-31% of cells in NPC-Chimeroids (FIG. 2K) vs. 76-95% and >99% of cells in NSC- and PSC-Chimeroids, respectively. Notably, GM still showed a higher proportion of proliferative cell types (aRG, oRG, and IP) compared to cells from other donors (34.4%-35.5% in GM vs 11.0%-23.8%; binomial mixed effects model, p<10⁻¹⁵; FIG. 2K), indicating that the Chimeroid protocol preserves intrinsic differences between donors. Overall, Chimeroid technology allows using neural progenitors at different stages of development as starting cell types for aggregation, and performing donor mixing at later stages of organoid development can overcome substantial donor-specific growth advantages, without requiring prior knowledge of line-specific properties.

### Cellular Diversification in NSC- and NPC-Chimeroids is Comparable to Single-Donor Organoids

To understand if the cell types produced in Chimeroids are comparable to those produced in single-donor organoids, the inventors compared the cell compositions produced by the same donor lines in the different models. To control for any effect of dissociation, the inventors generated single-donor NSC-Chimeroids by dissociation and reaggregation of nascent organoids from a single donor line. The inventors profiled 53,904 cells from individual NSC-Chimeroids (from two different mixes, Mix 1 and Mix 2), 4,132 cells from NPC-Chimeroids, and 35,180 cells from single-donor Chimeroids, all at 3 mo, and compared them to the published single-cell atlas of cortical organoid development (Uzquiano et al., Cell, 2022).

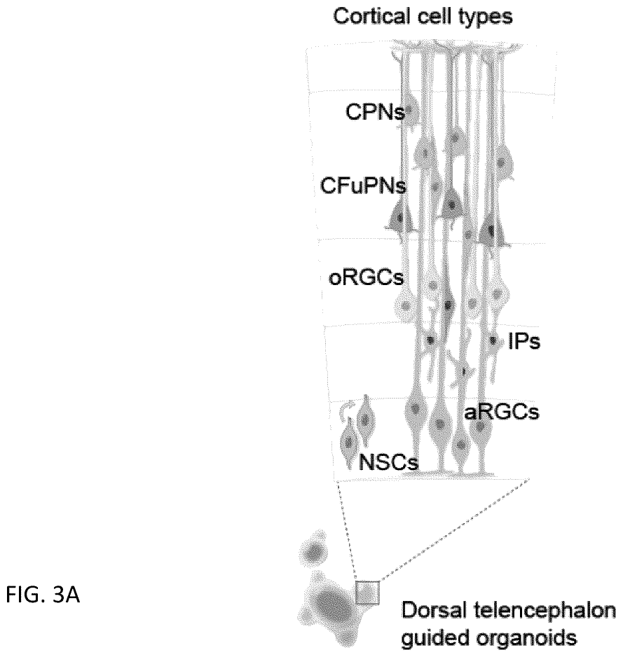

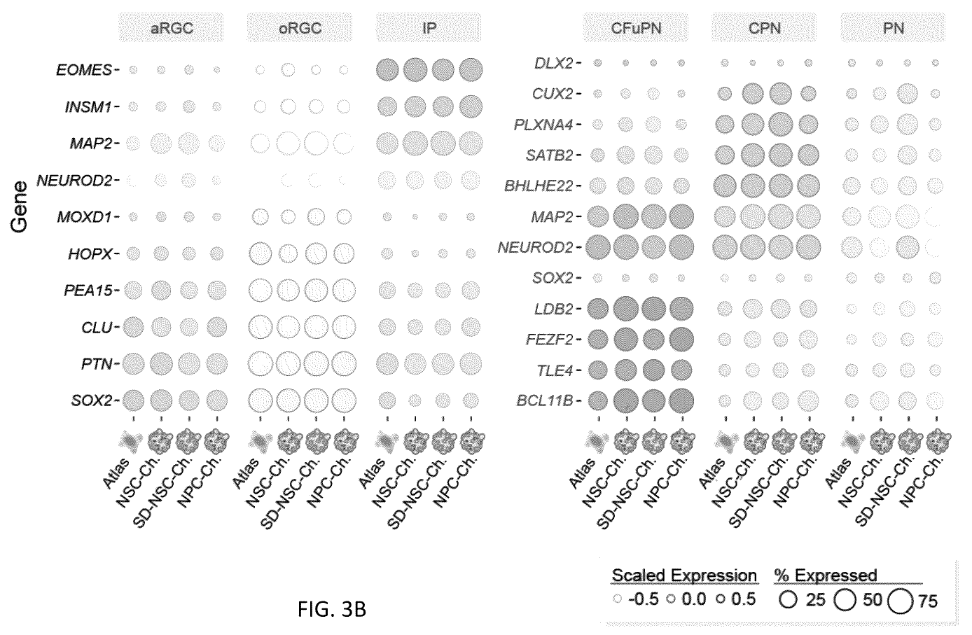

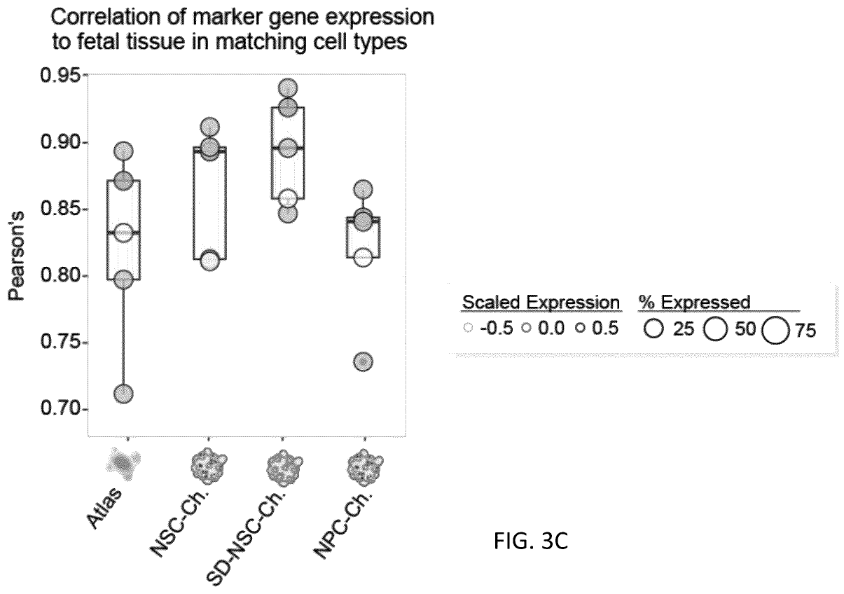

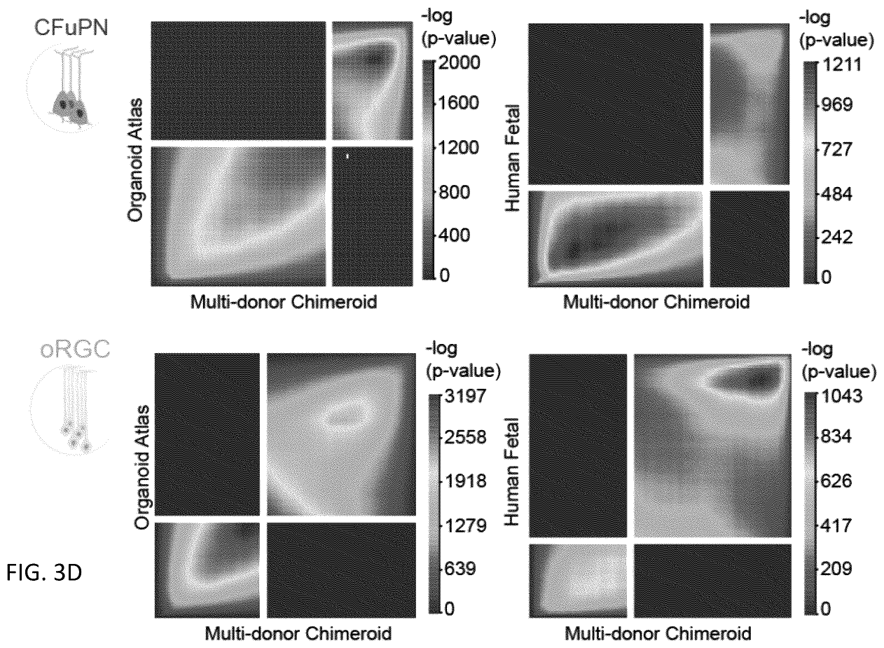

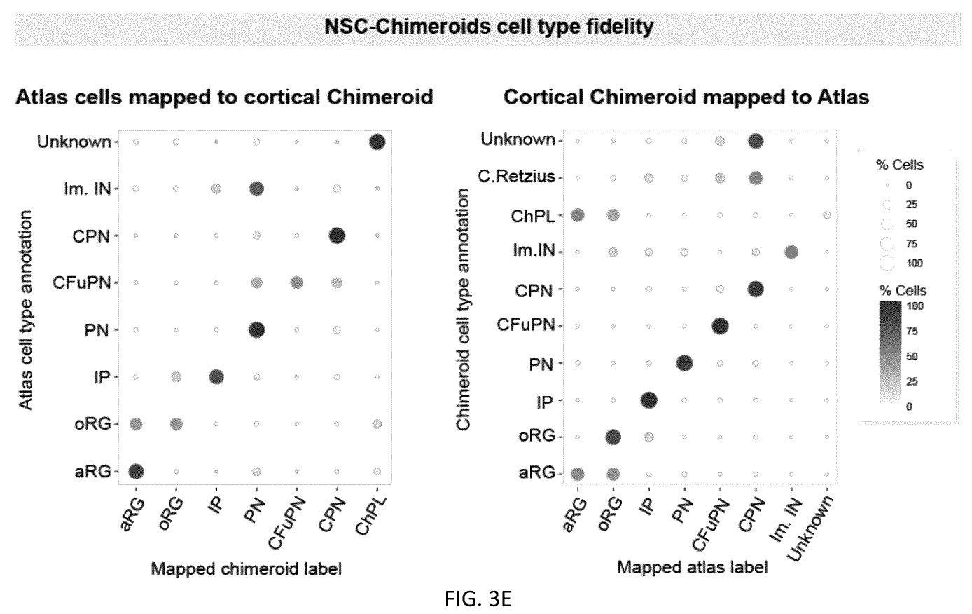

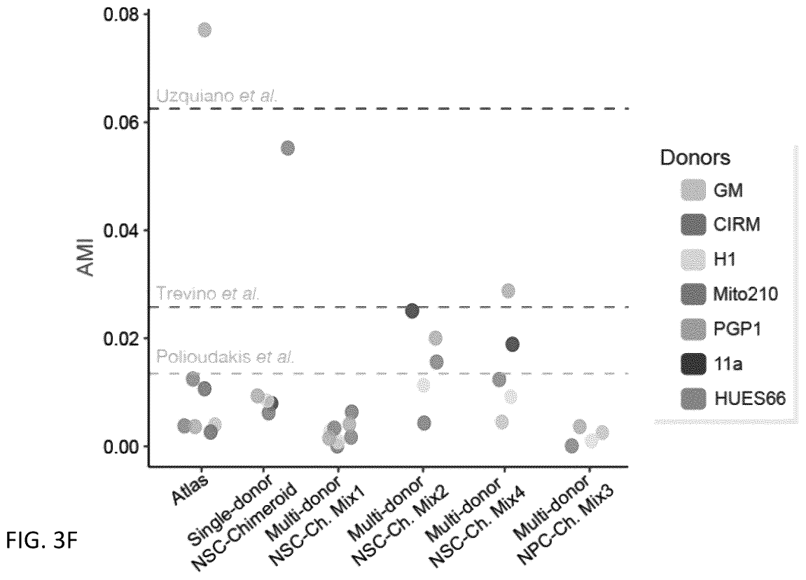

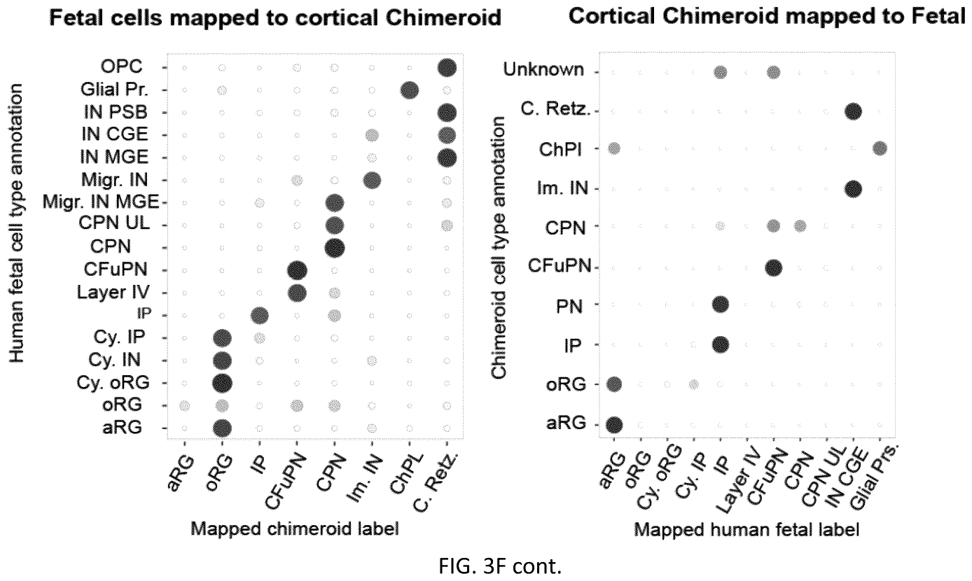

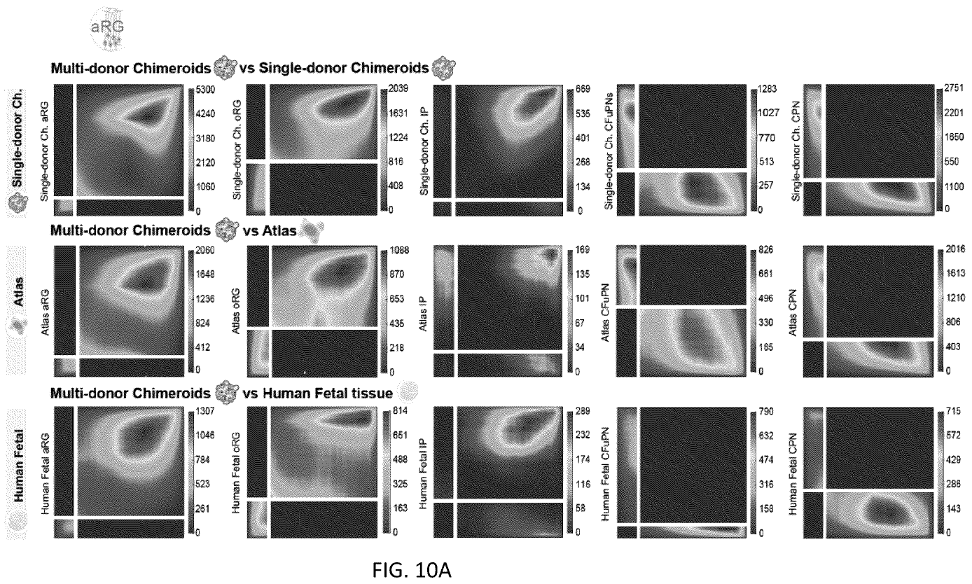

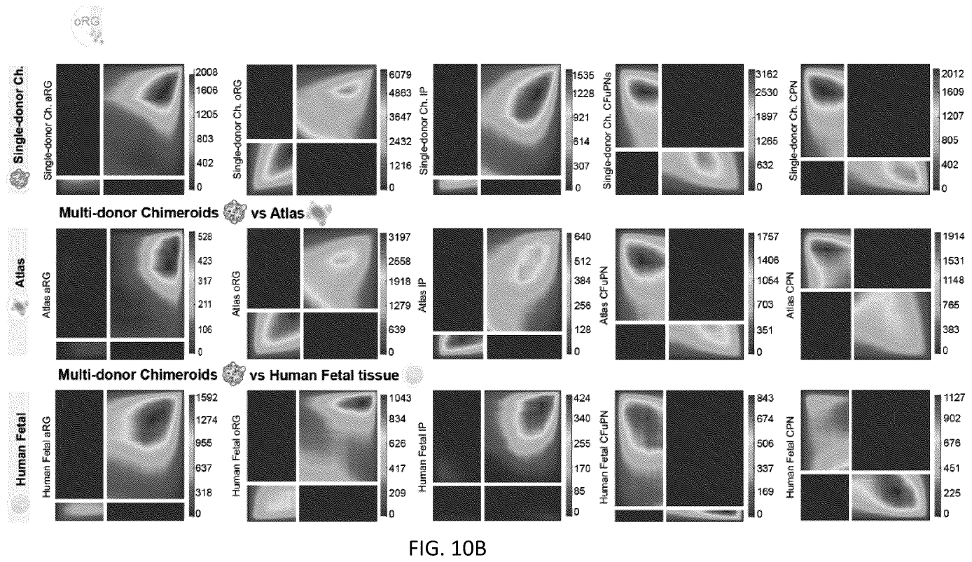

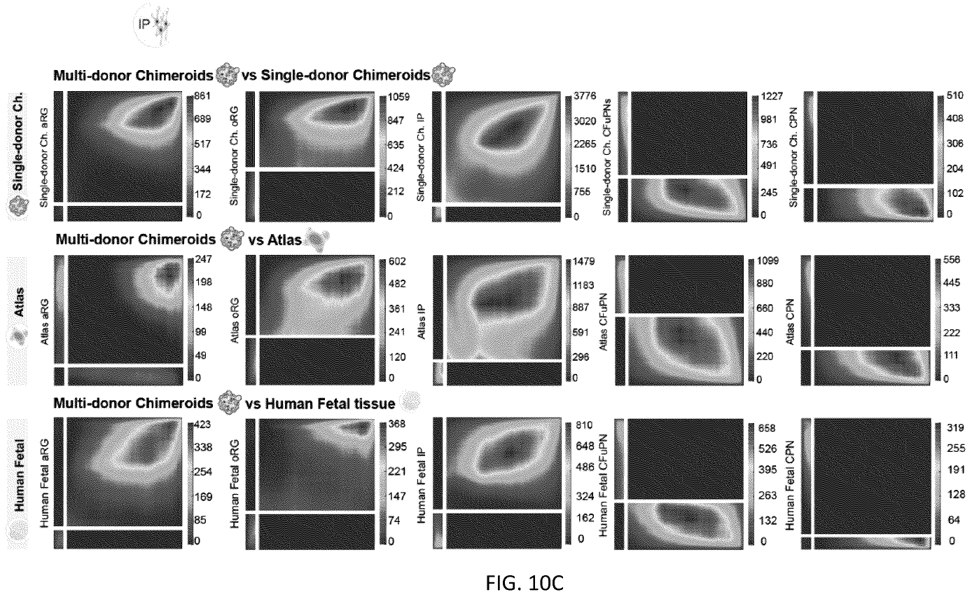

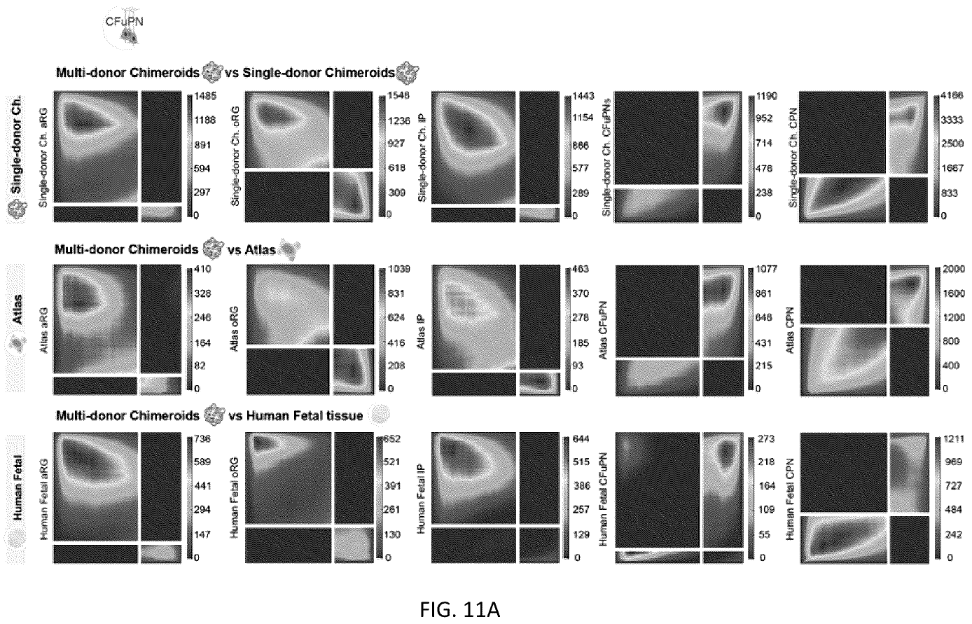

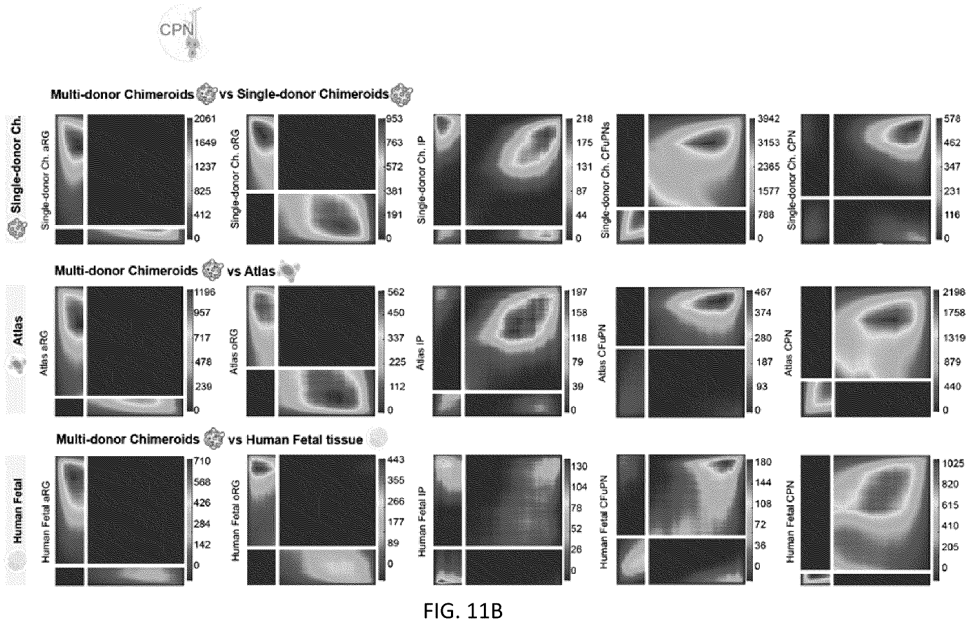

All multi-donor Chimeroids contained the same cell type subsets present in 3 mo single-donor organoids from the published reference atlas, including apical radial glia (aRG), outer radial glia (oRG), intermediate precursor cells (IPC), and corticofugal (CFuPN) and callosal (CPN) projection neurons (FIGS. 3A-3C). Moreover, the expression profiles of 19 canonical cell-type marker genes for key cell populations were highly concordant between Chimeroids and single-donor organoids from the atlas (Pearson correlation coefficients for normalized marker gene expression between corresponding cell types ranging 0.93-0.98; FIG. 3B), and between corresponding cell types of Chimeroids and fetal cortex (Pearson correlation coefficients ranging 0.81-0.91; FIG. 3C). The correlation between expression of these marker genes in NSC-Chimeroids and fetal cortex was not significantly different from the correlations between single-donor atlas organoids and fetal cortex (paired Wilcoxson test, p=0.19) or between single-donor NSC-Chimeroids and fetal cortex (p=0.06), indicating that Chimeroid cell types demonstrate equally high similarity to endogenous cell types. There was also a high degree of global similarity between expression profiles in corresponding cell types in the NSC-Chimeroids compared to both the organoid atlas (Uzquiano et al., Cell, 2022) and endogenous fetal tissue (rank-rank hypergeometric overlap (RR-HO) (Cahill et al., Sci Rep, 2018, Methods, FIG. 3D, FIGS. 10-11).

Finally, reference label transfer (Stuart et al., Cell, 2019) from NSC-Chimeroids to cell types from two reference datasets, endogenous human fetal cortex and single-donor organoids, or vice versa (Methods), showed that Chimeroid cells were predominantly assigned to the expected reference cell type in both datasets, indicating high similarity in expression profiles (FIG. 3E).

To assess Chimeroid-to-Chimeroid variability in cell-type composition, the inventors calculated the adjusted mutual information (AMI) (Xuan Vinh et al., Journal of Machine Learning Research, 2010) of cell-type abundances between replicates within each batch of organoids from the atlas, as well as between replicates for each donor in each batch of single-donor and multi-donor NSC-Chimeroids. AMI scores for multi-donor NSC-Chimeroids or for single-donor Chimeroids were not significantly different from those for atlas organoids (Wilcoxon rank-sum test, p=0.98 and p=0.53, respectively) (FIG. 3F), indicating that Chimeroids retain high chimeroid-to-chimeroid reproducibility comparable to that of the established cortical organoid protocol (FIG. 3F). Notably, 3-mo Chimeroid replicates had AMI scores comparable to those of individual human fetal brains profiled in published datasets (Polioudakis et al., Neuron, 2019; Trevino et al., Cell, 2021; Uzquiano et al., Cell, 2022) (FIG. 3F, dashed lines). Interestingly, within individual multi-donor Chimeroids, the variability in cell-type composition between donors was low (mean AMI=0.0096), and not different from the organoid-to-organoid AMI found in single-donor cortical organoids (Wilcoxon test p=0.9), indicating that donor-specific differences in cell type composition are consistent across individual Chimeroids.

Overall, these data show that the Chimeroid model is reproducible between replicates of the same mixture of donors and is concordant with the original single-donor organoid model. Multi-donor Chimeroids also retain the same degree of cellular complexity and molecular resemblance to endogenous human fetal tissue as the original cortical organoid model (Uzquiano et al., Cell, 2022; Velasco et al., Nature, 2019).

### Ethanol and Valproic Acid Induce Cell Type-Specific Effects in Chimeroids

To demonstrate the utility of the Chimeroid model to investigate inter-individual variability in response to perturbation, the inventors tested the effects of two neurotoxic triggers that cause neurodevelopmental abnormalities: ethanol (EtOH) and valproic acid (VPA). Fetal exposure to EtOH in humans can result in FASD (Alfonso-Loeches and Guerri, Crit Rev Clin Lab Sci, 2011; Arzua et al., Transl Psychiatry, 2020; Carpita et al., Brain Sci, 2022; Charness, Alcohol Res, 2022; Eberhart and Parnell, Alcohol Clin Exp Res, 2016; Granato and Dering, Int J Mol Sci, 2018; Marguet et al., Acta Neuropathol Commun, 2022; Streissguth and Dehaene, Am J Med Genet, 1993; Sulik et al., Science, 1981; Williams et al., Pediatrics, 2015; Wozniak et al., Lancet Neurol, 2019), with high inter-individual variation in clinical phenotype (Eberhart and Parnell, Alcohol Clin Exp Res, 2016). Valproic acid (VPA), used to treat epilepsy and psychiatric disorders, is associated with augmented risk of developing ASD in offspring when administered during pregnancy (Bjørk et al., JAMA Neurol, 2022; Christensen et al., JAMA, 2013).

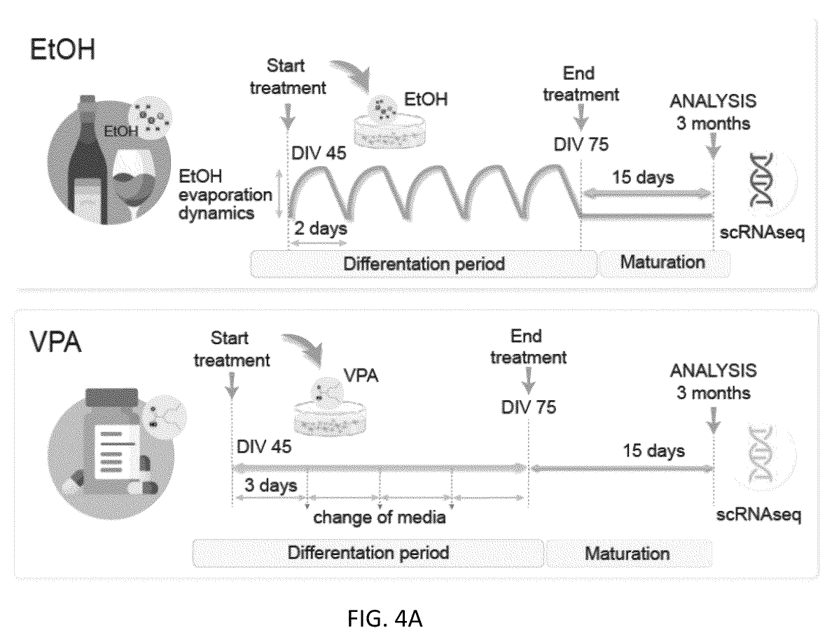

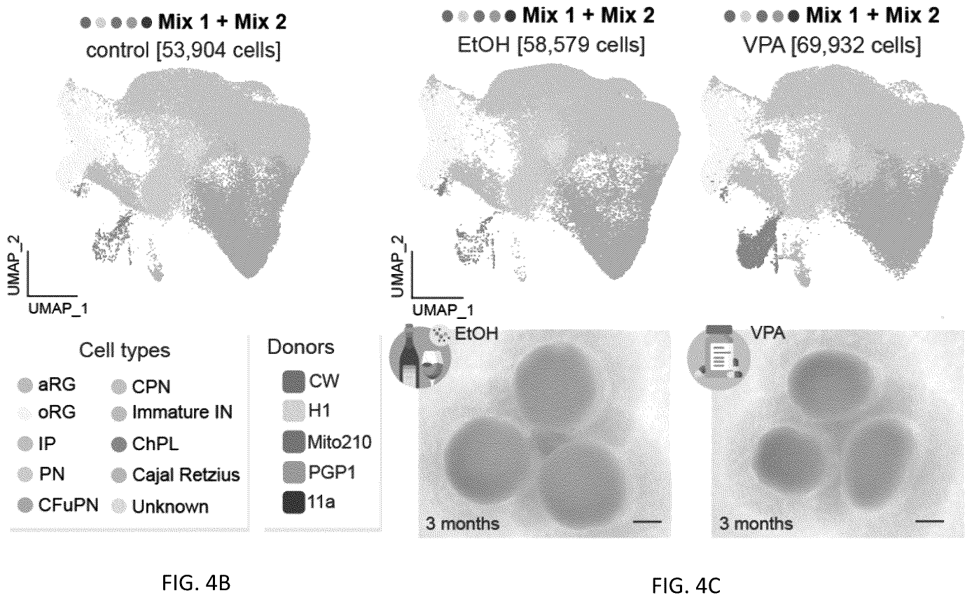

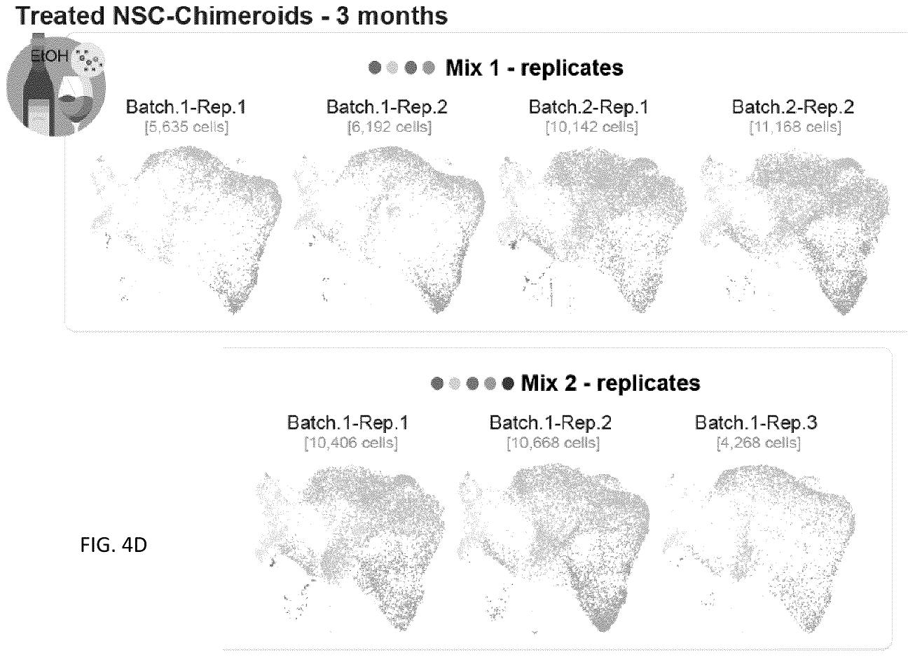

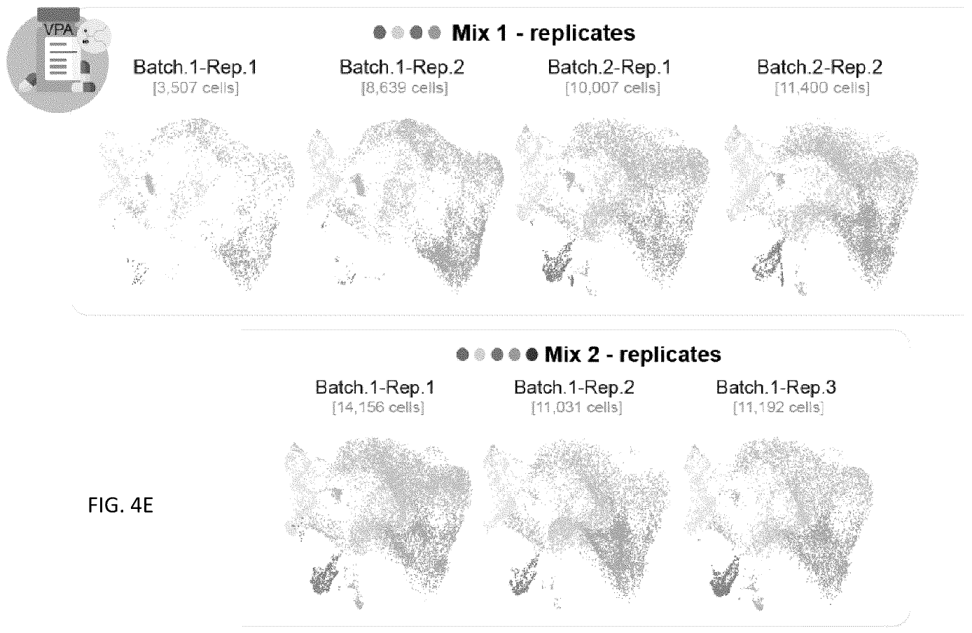

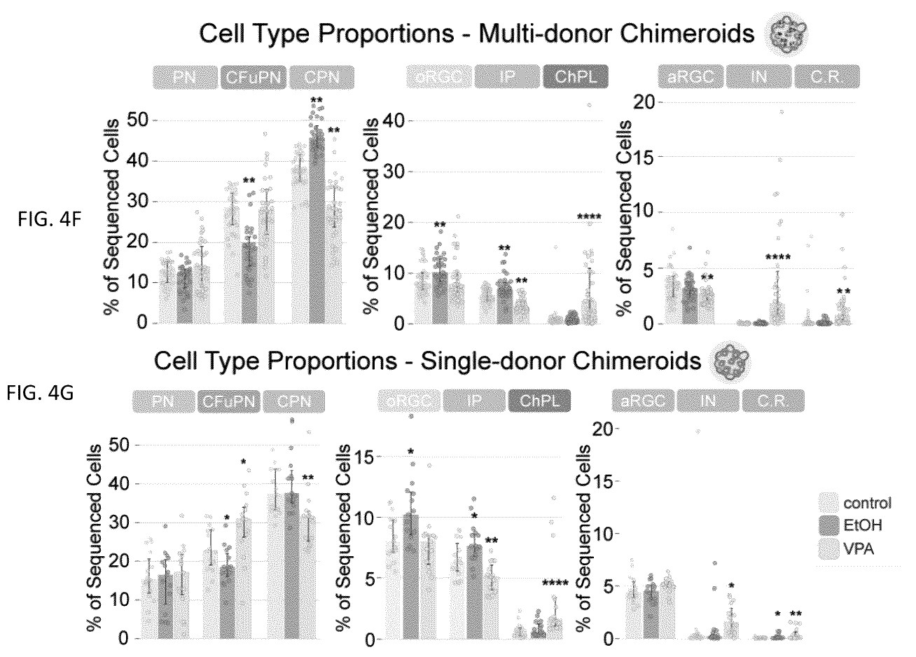

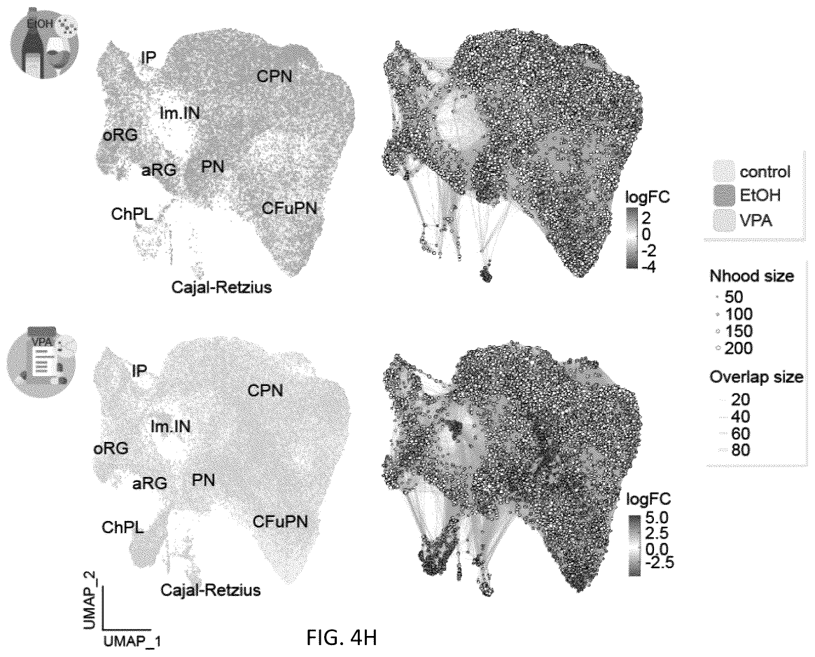

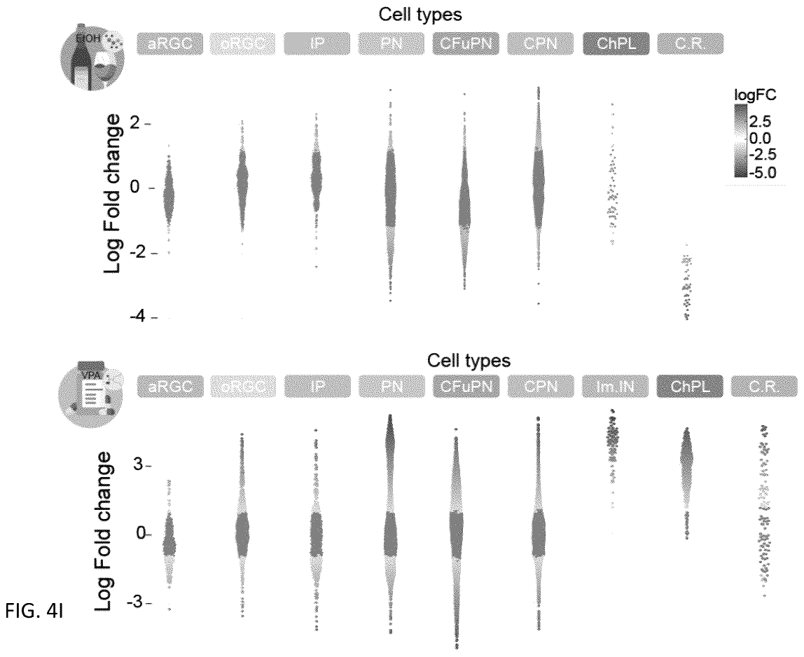

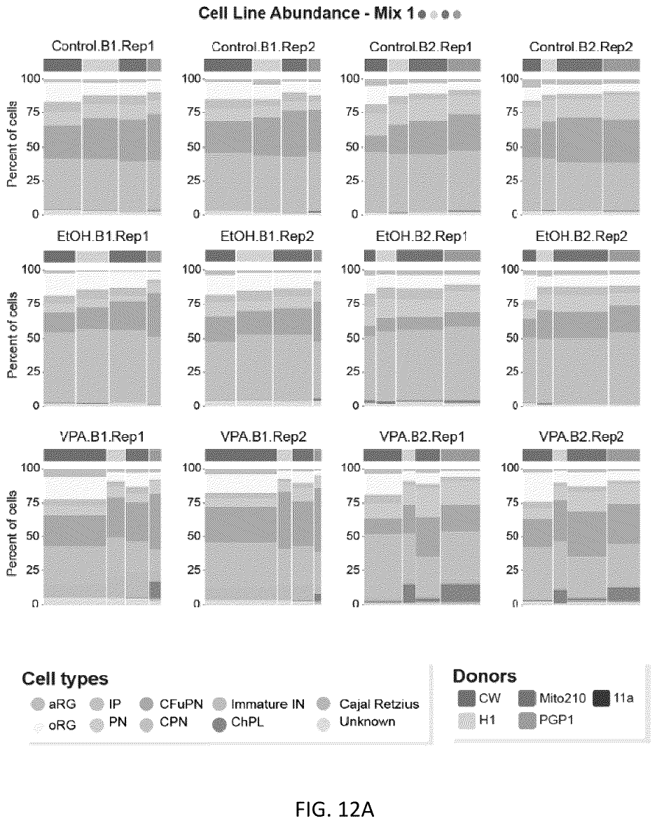

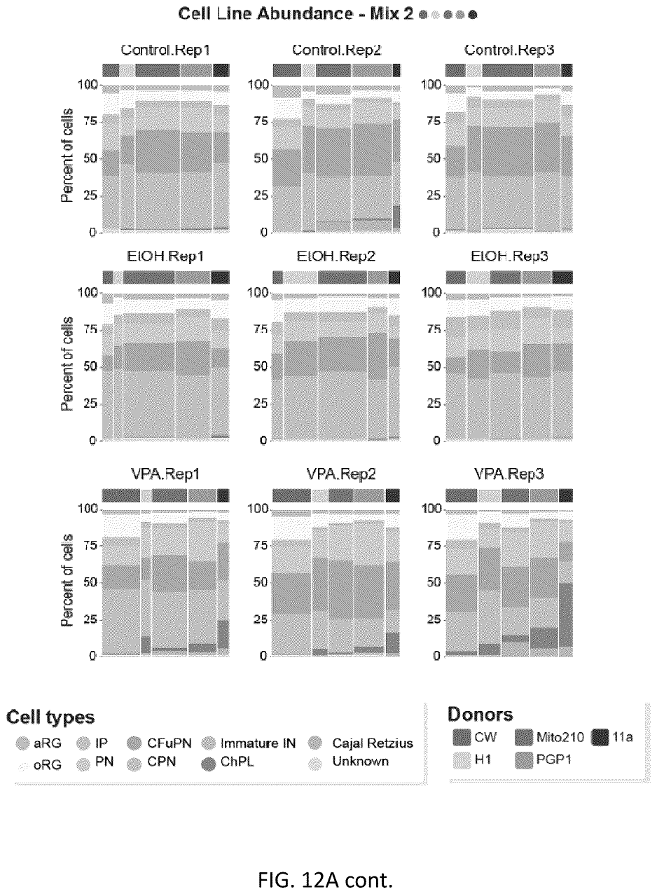

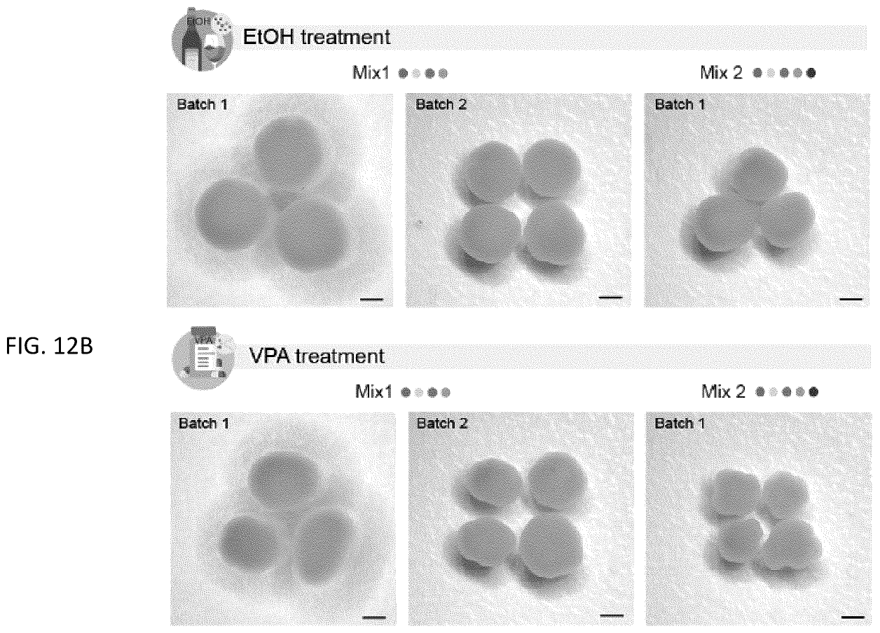

The inventors generated NSC-Chimeroids from two different mixes, Mix 1 and Mix 2, and treated them with either EtOH or VPA for 30 days. To model physiological fluctuations typical of alcohol exposure during pregnancy, the inventors administered 50 μM of EtOH to the culture media every two days, from DIV45 to DIV75, with plates sealed for 24 hours after EtOH addition to avoid evaporation (Arzua et al., Transl Psychiatry, 2020) (FIG. 4A, top). To mimic environmental exposure to VPA, the inventors added VPA at clinical concentrations (0.7 mM) directly to the culture media two times per week, from DIV45 to DIV75 (FIG. 4A, bottom). Both treatments were stopped at DIV75, and Chimeroids were cultured until DIV90. Finally, the inventors profiled the treated 3-mo NSC-Chimeroids from two donor mixes by scRNA-seq. The inventors jointly annotated the cells from both treatment groups, as well as the untreated Mix 1 and Mix 2 NSC-Chimeroids controls (above), by combining all VPA, EtOH, and control data, followed by clustering (Methods), and annotated cell clusters by expression of canonical marker genes and examination of genes upregulated in each cluster (Mix1 94,009 cells from n=12 Chimeroids, and Mix2 88,406 cells from n=9 Chimeroids; FIGS. 4B-4E, FIG. 12).

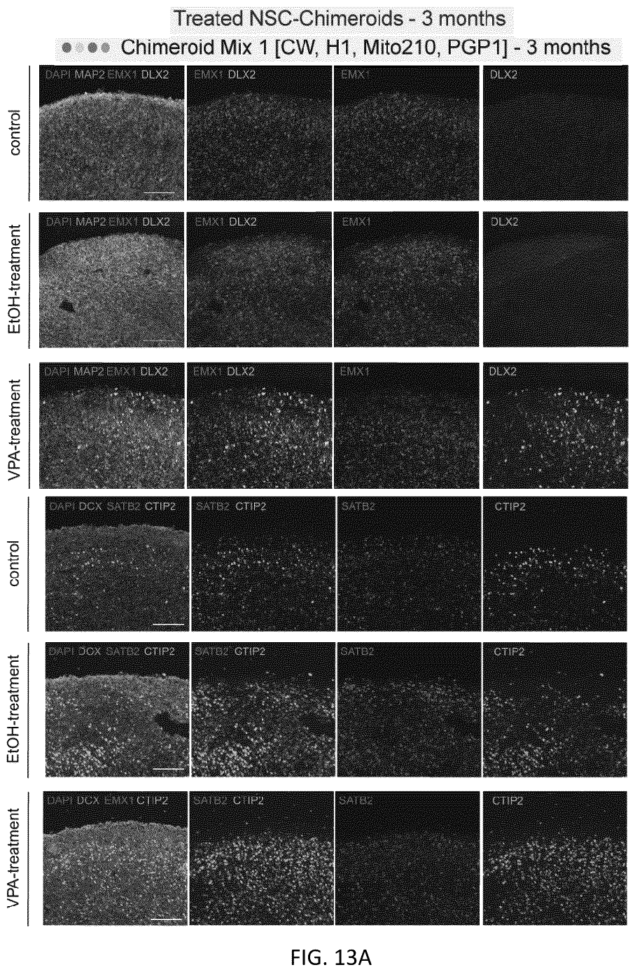

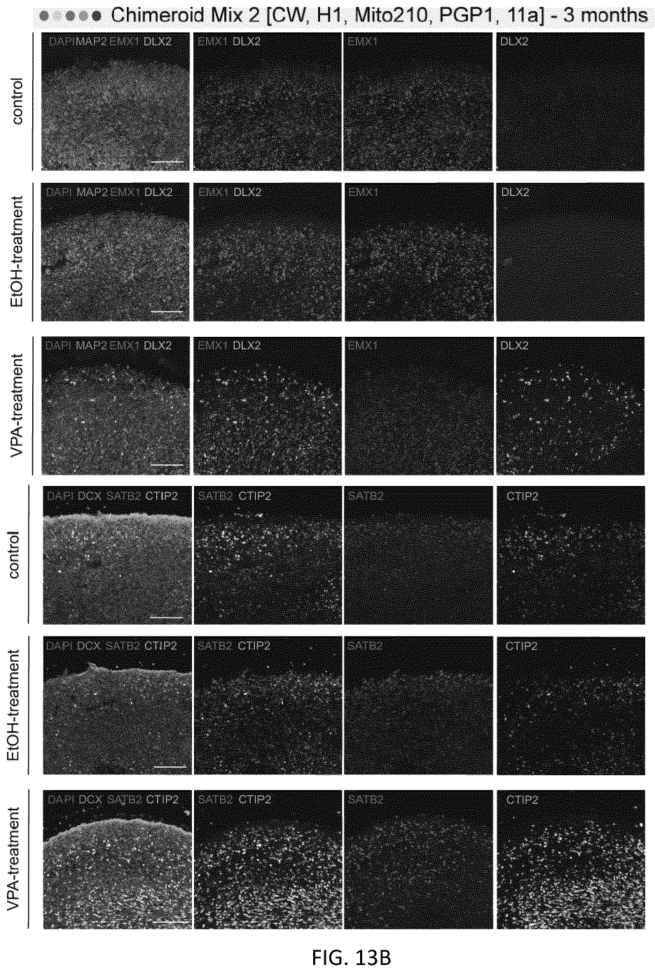

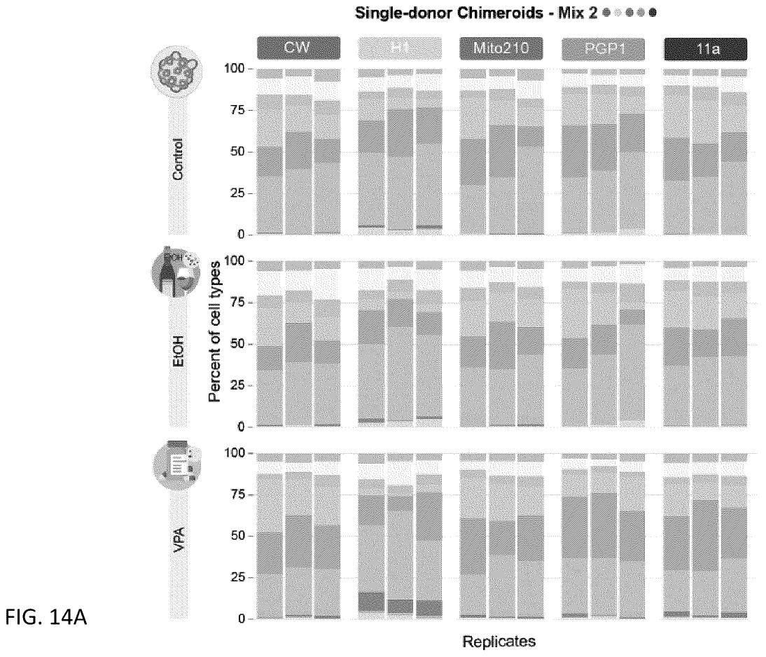

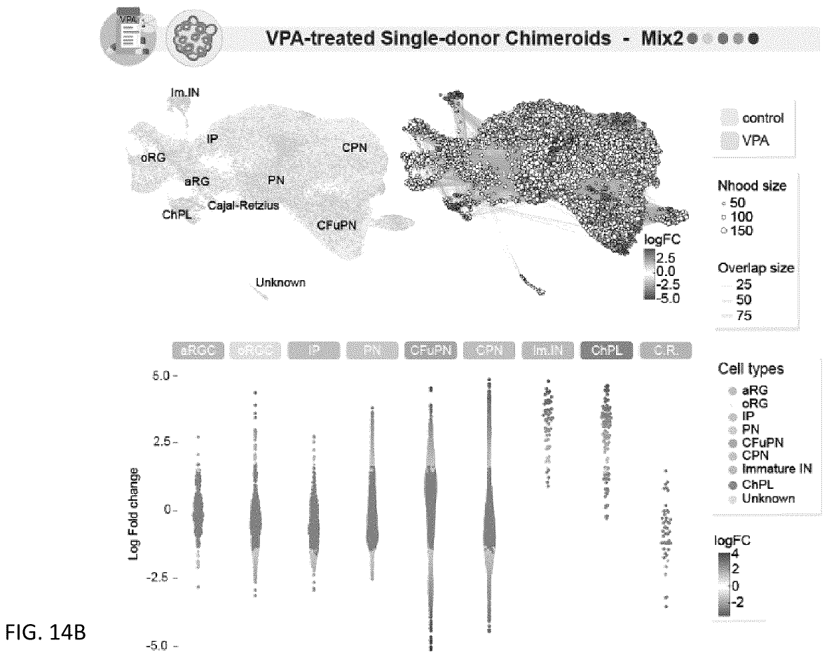

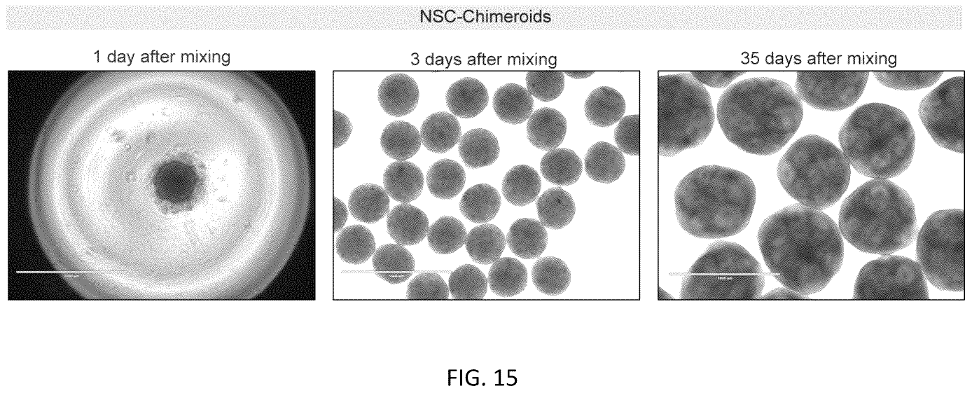

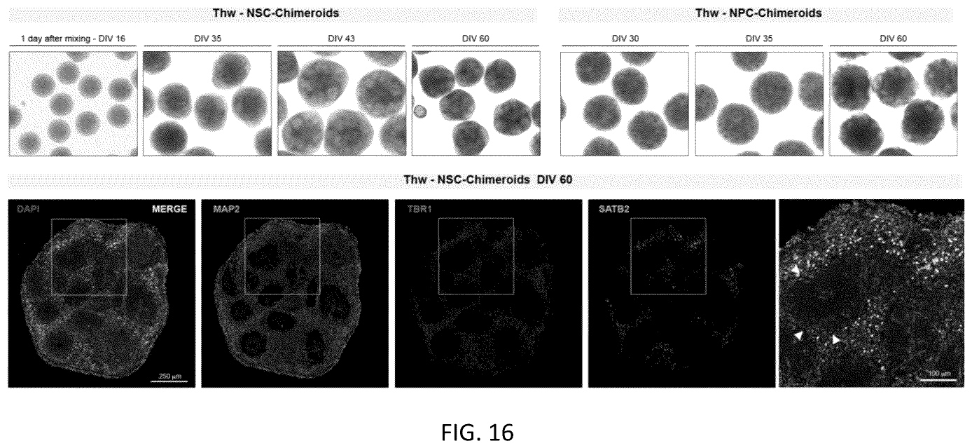

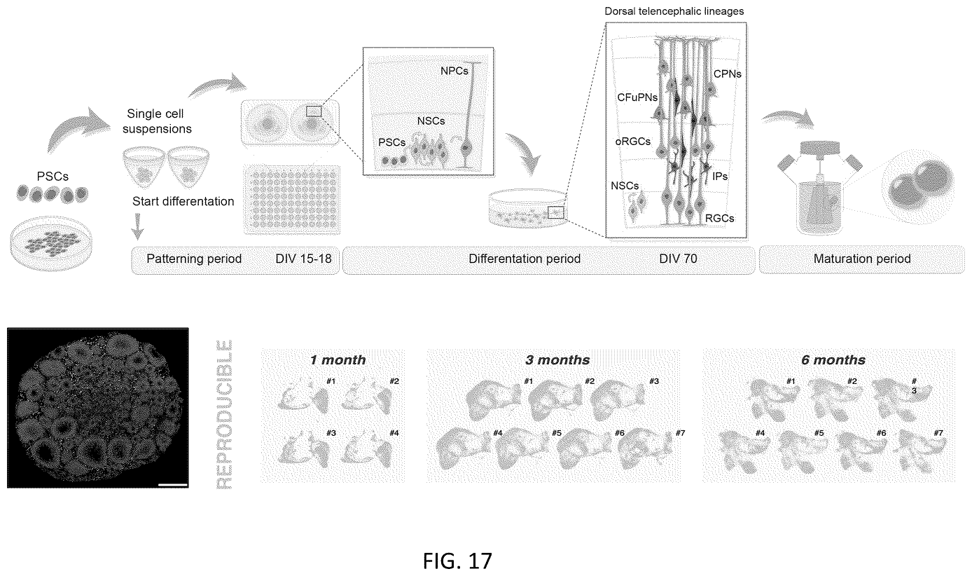

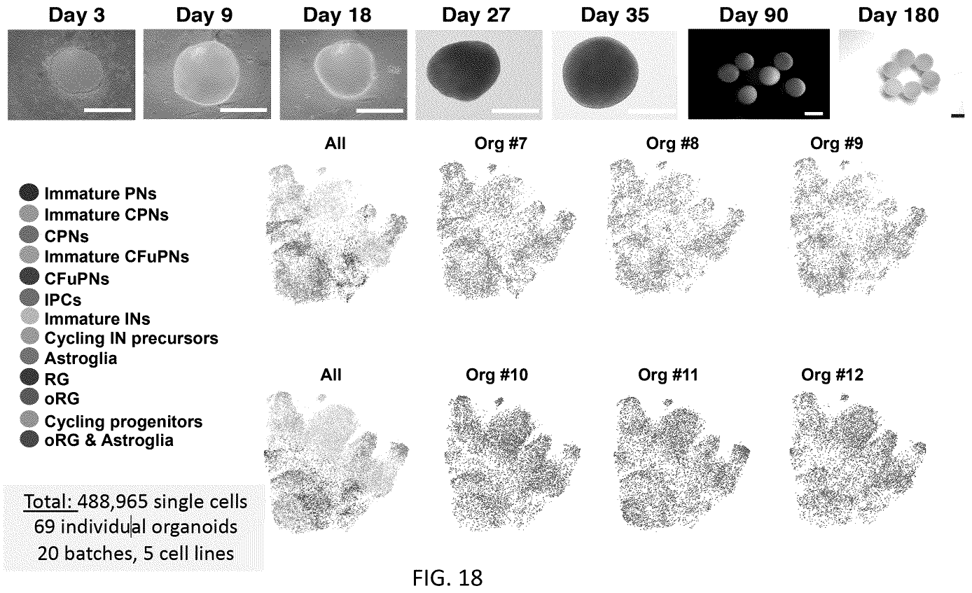

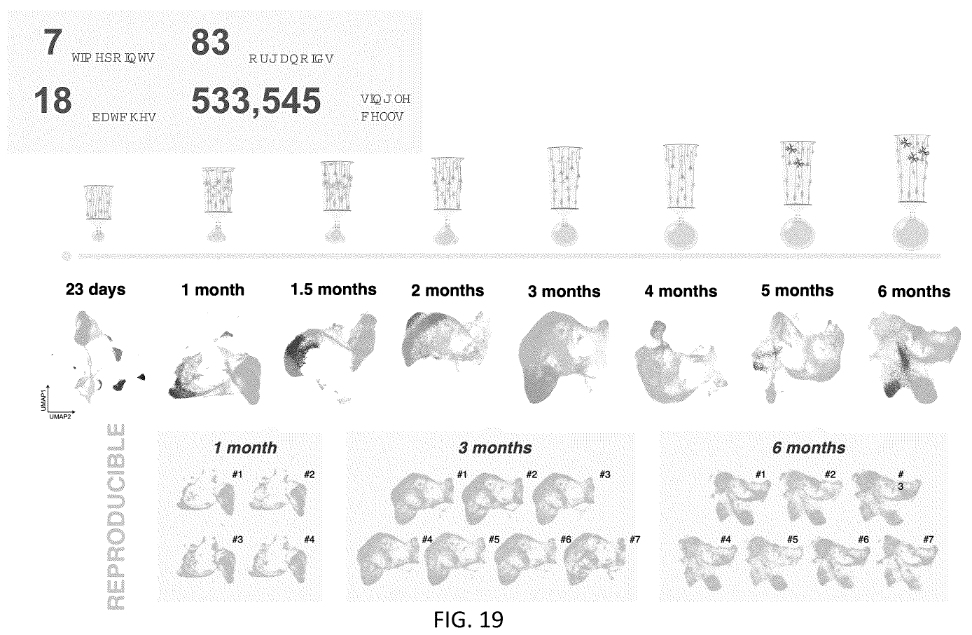

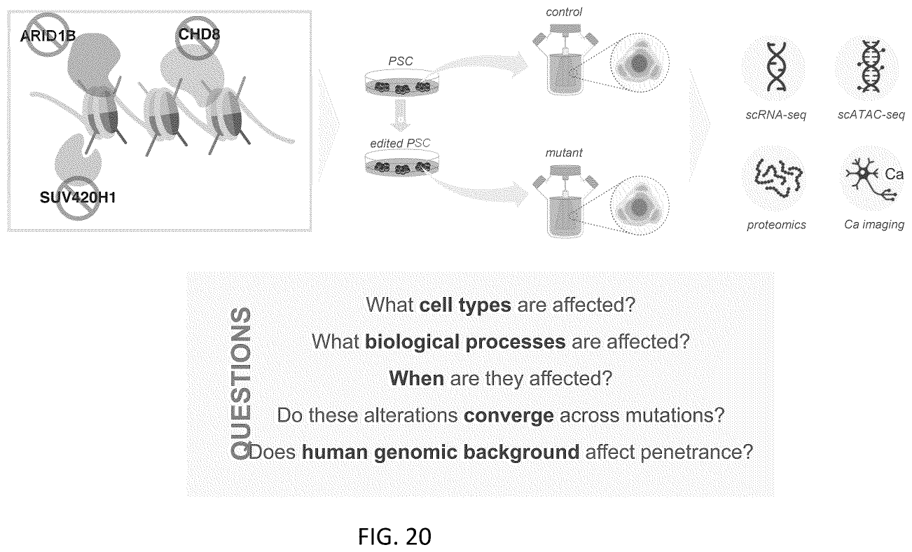

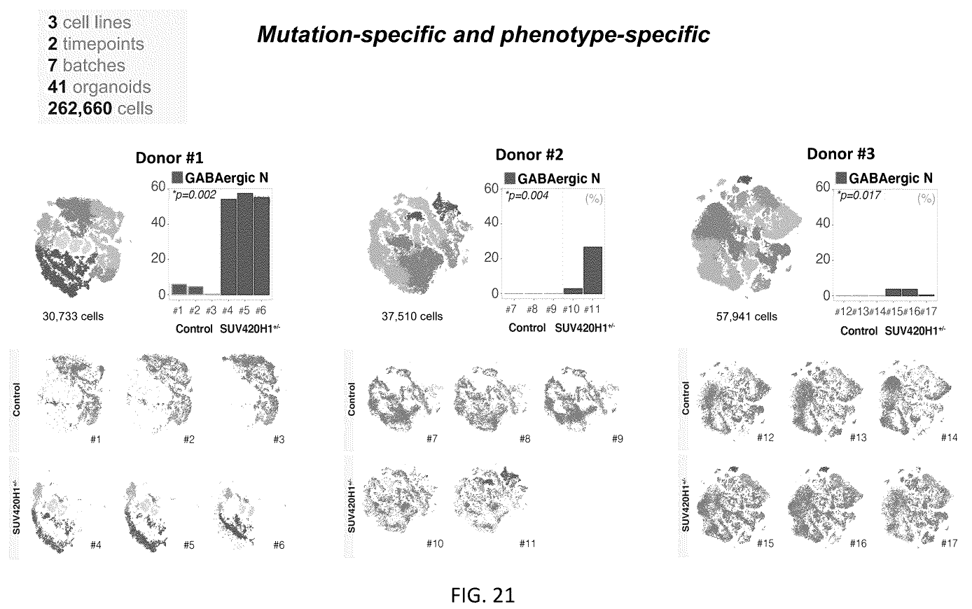

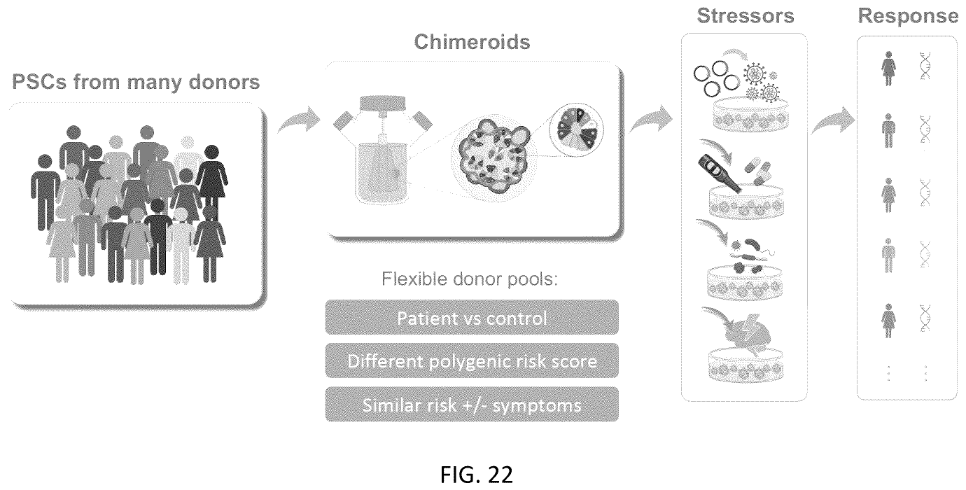

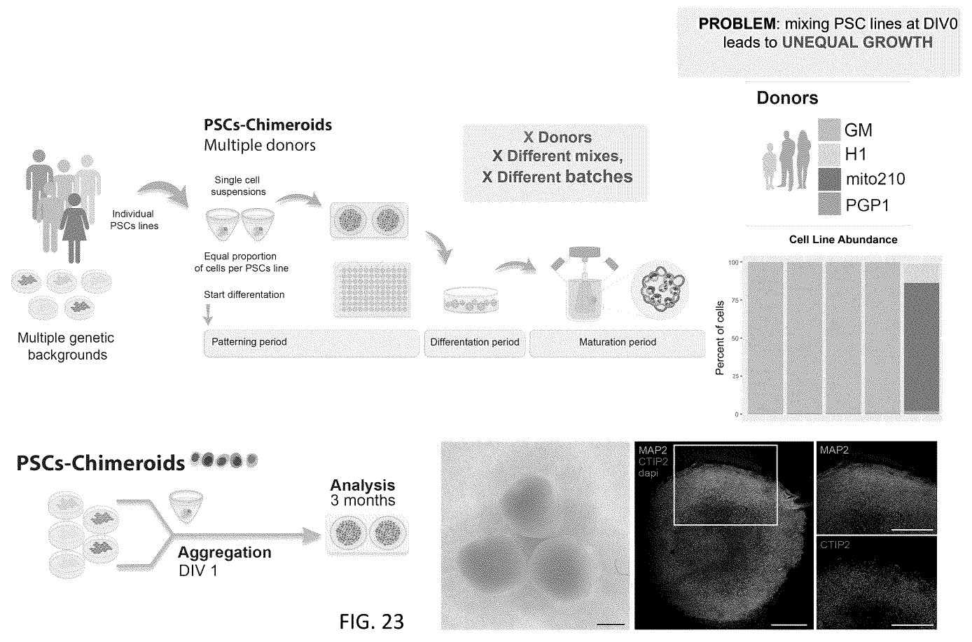

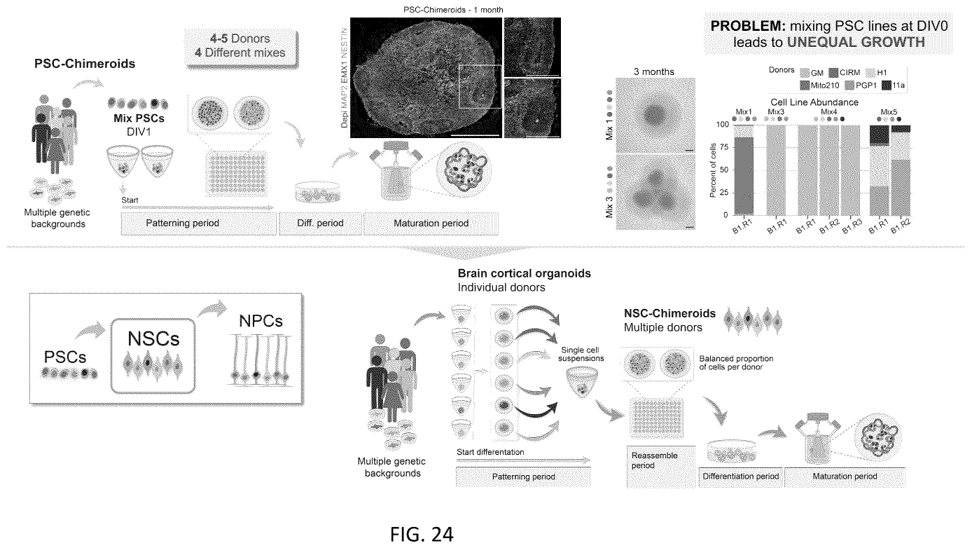

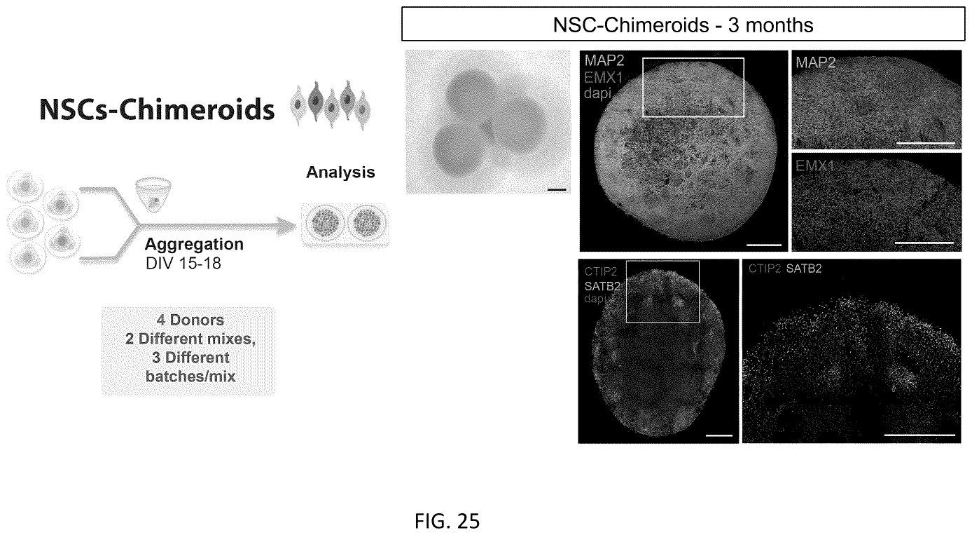

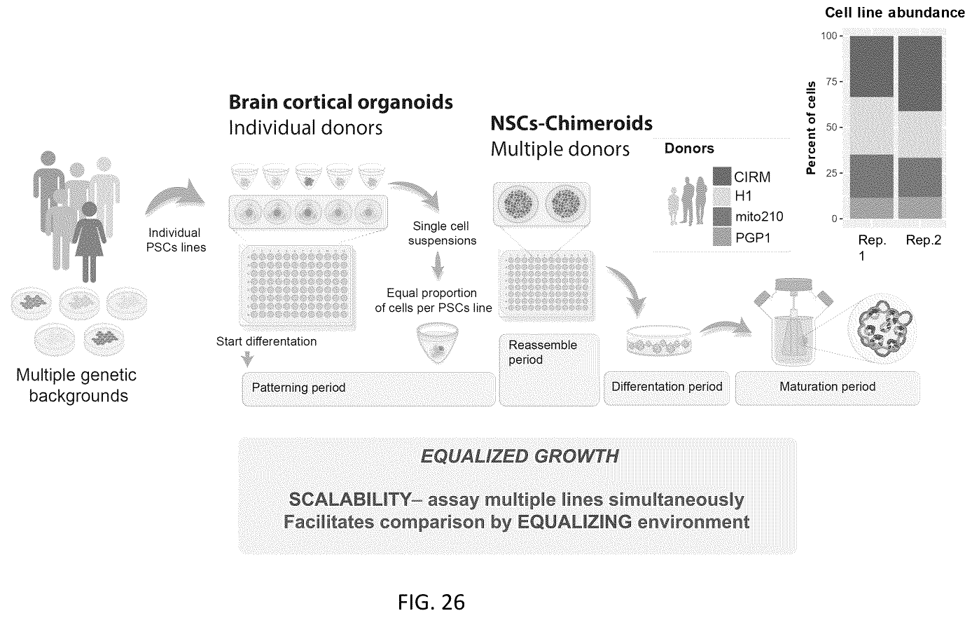

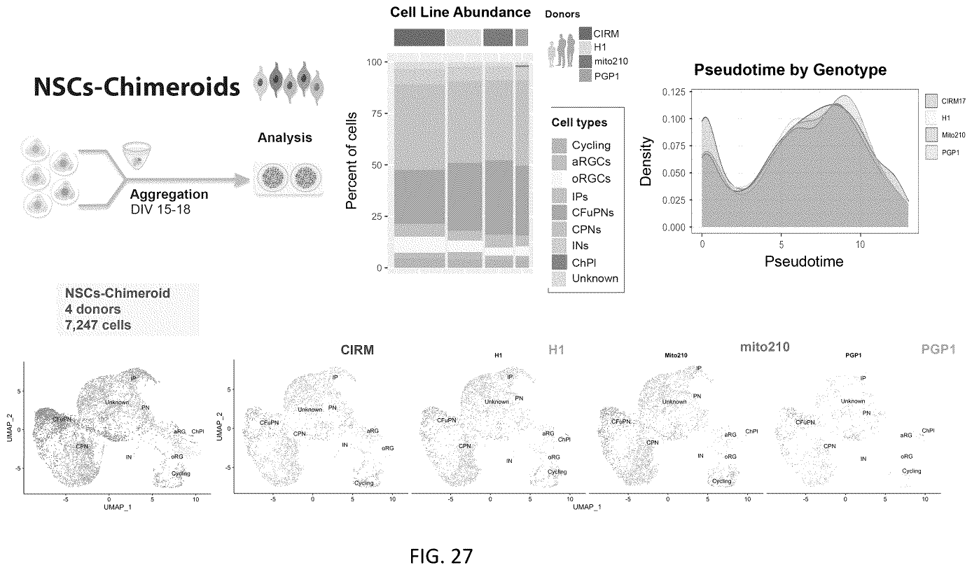

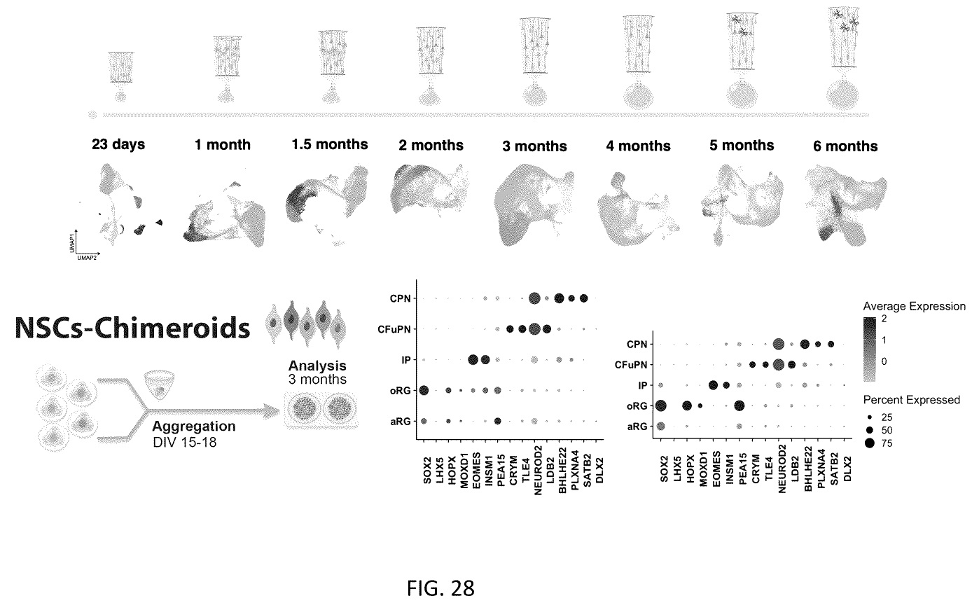

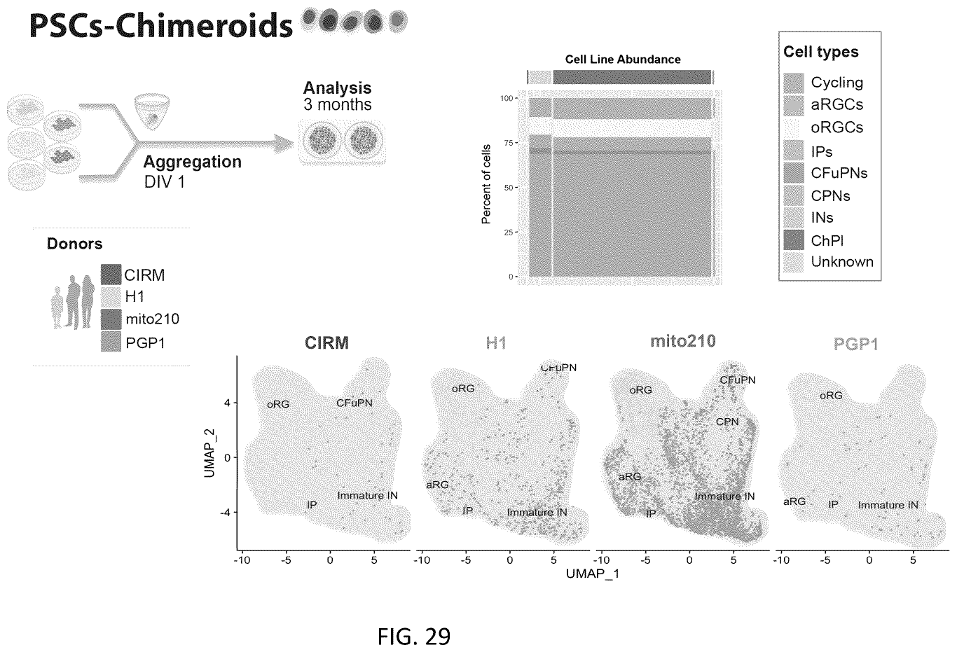

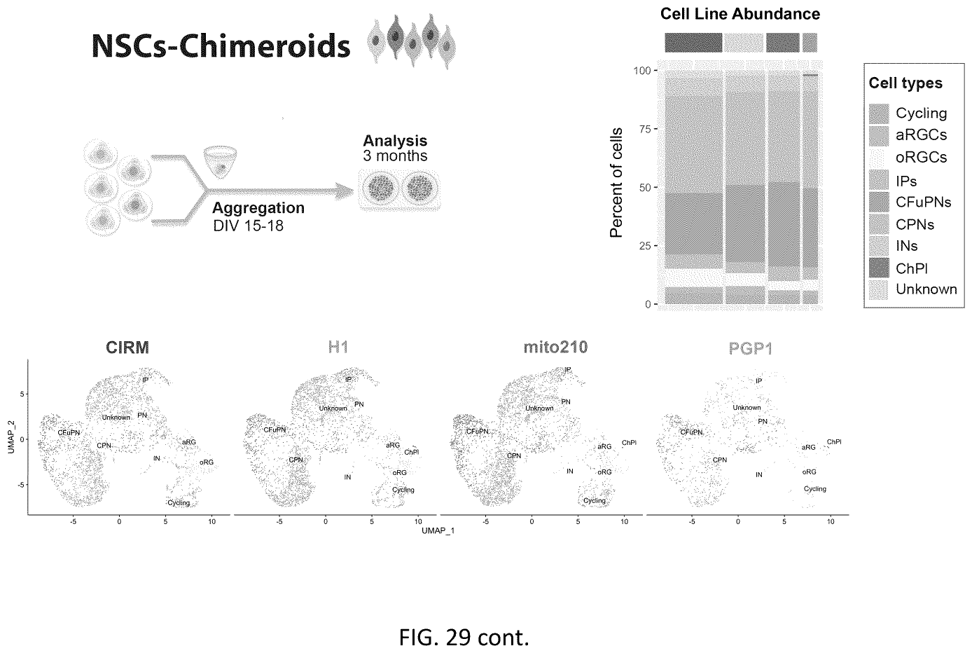

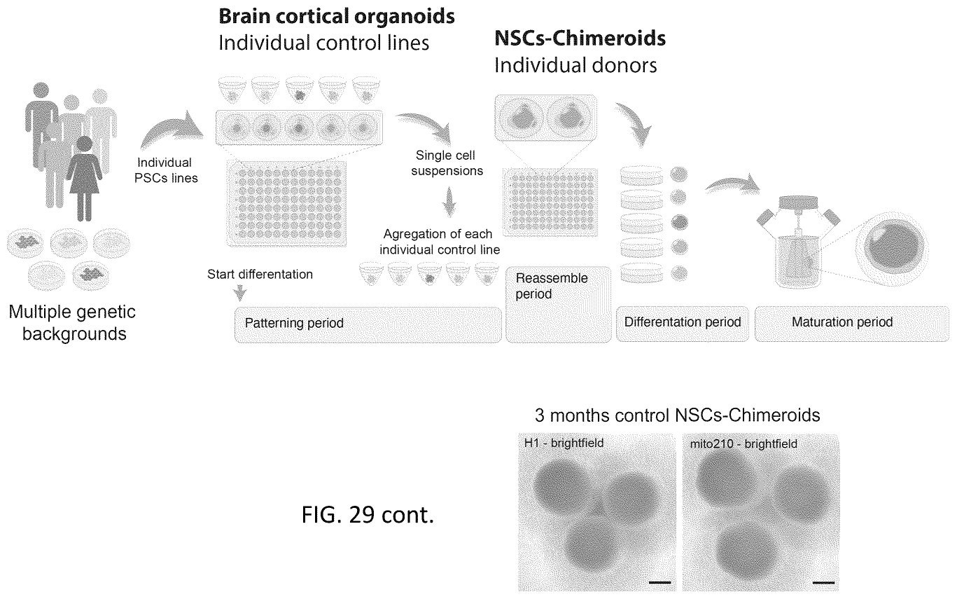

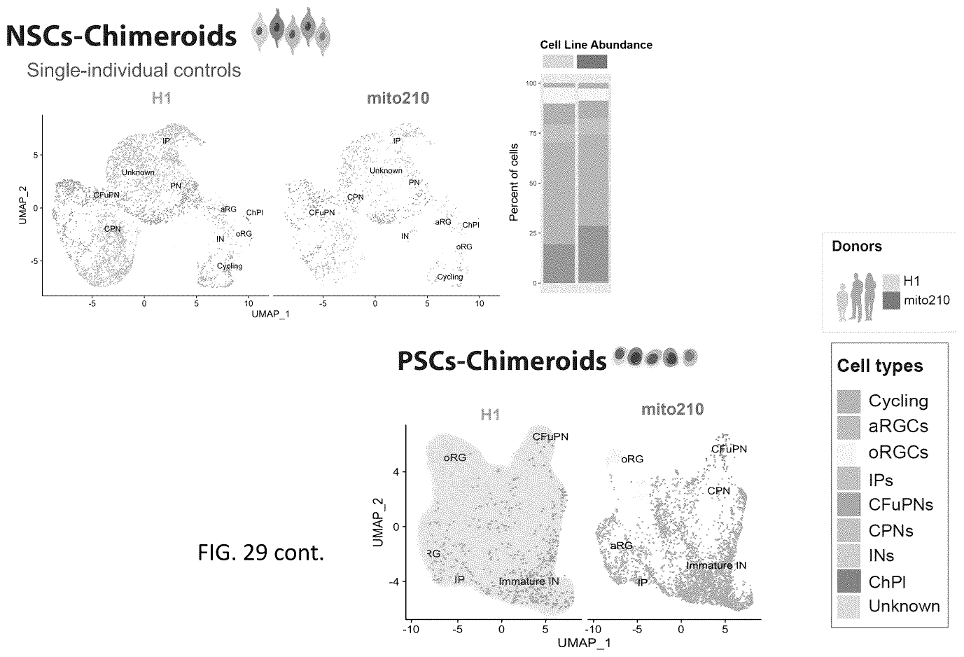

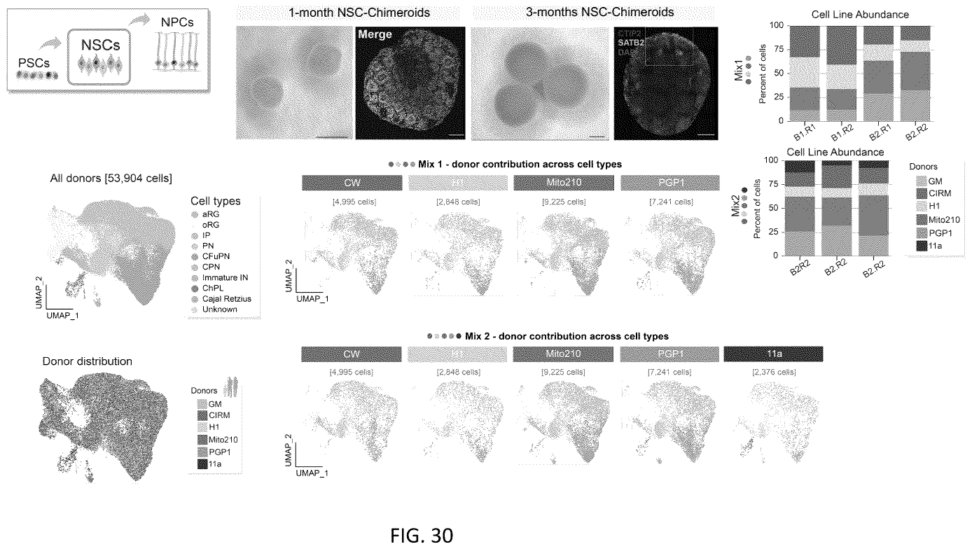

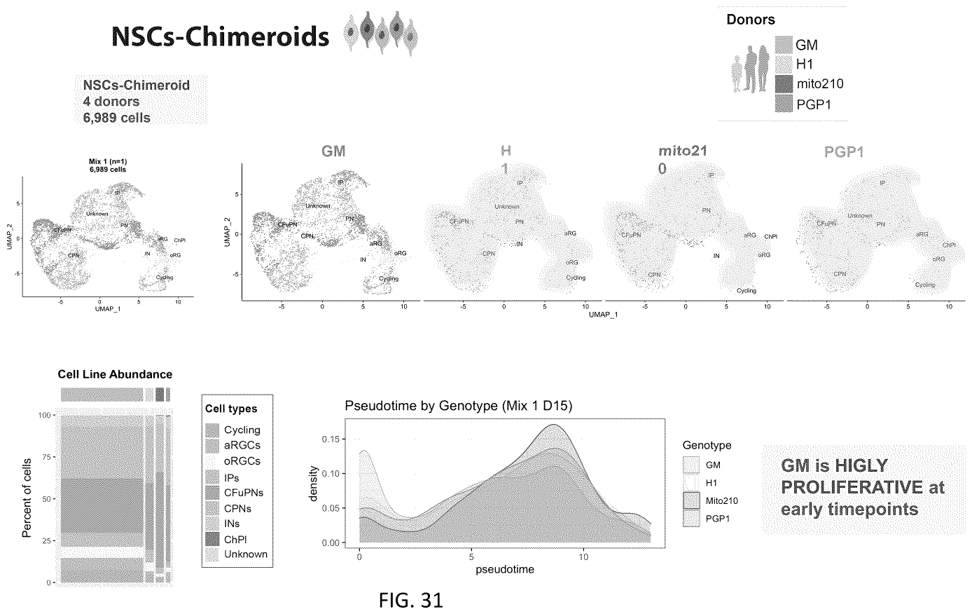

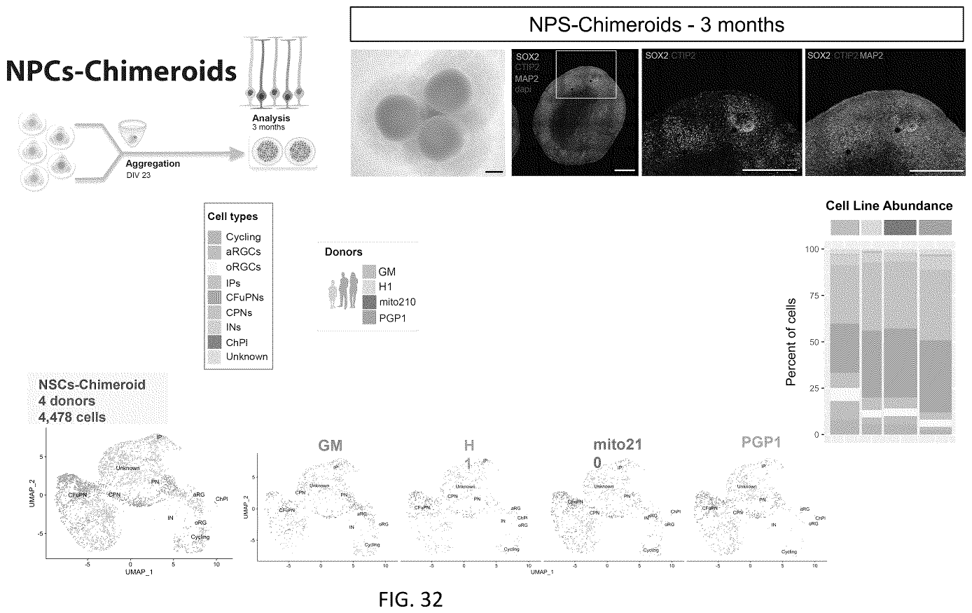

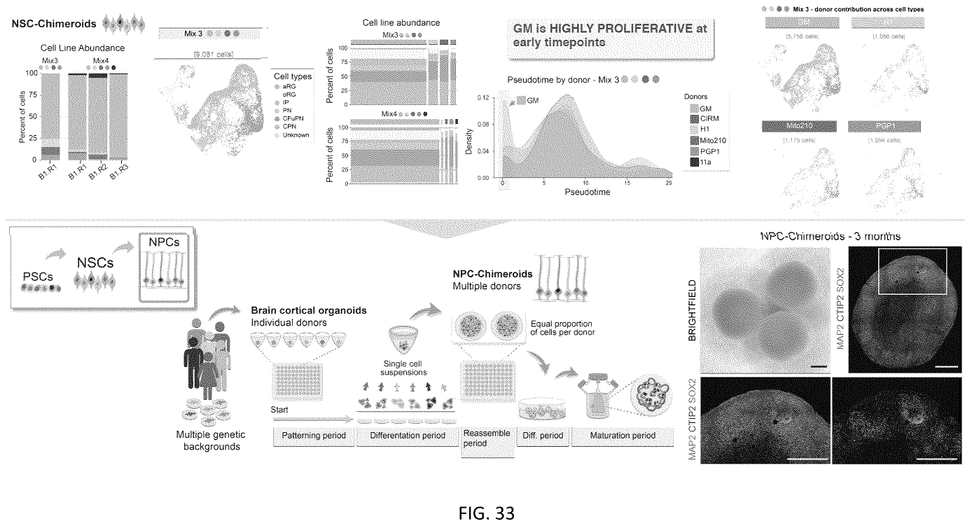

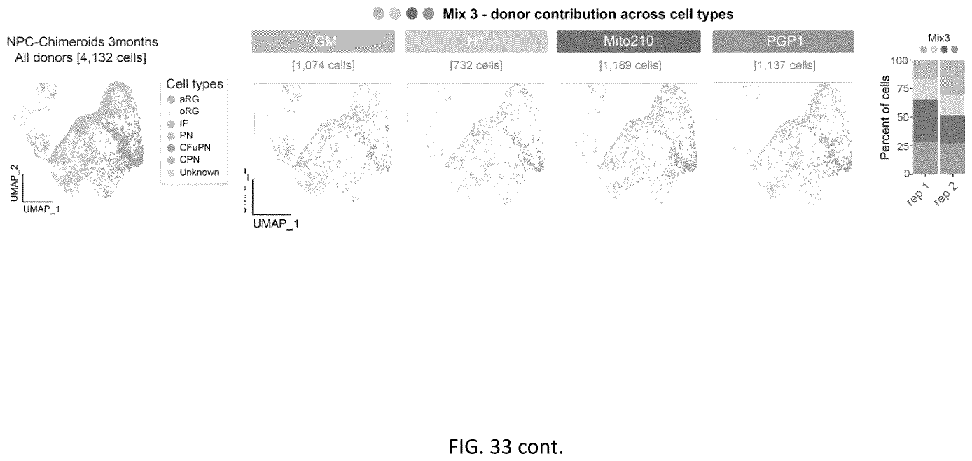

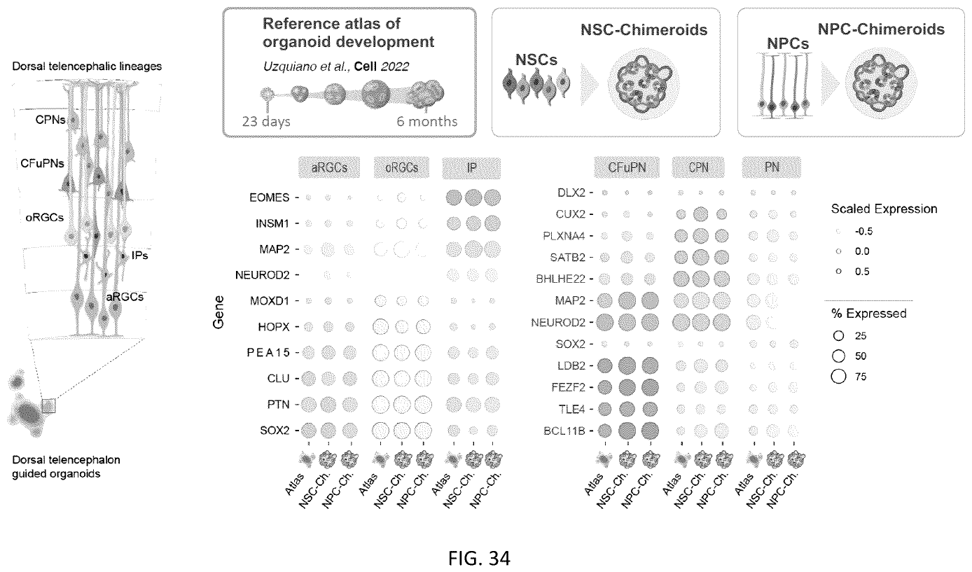

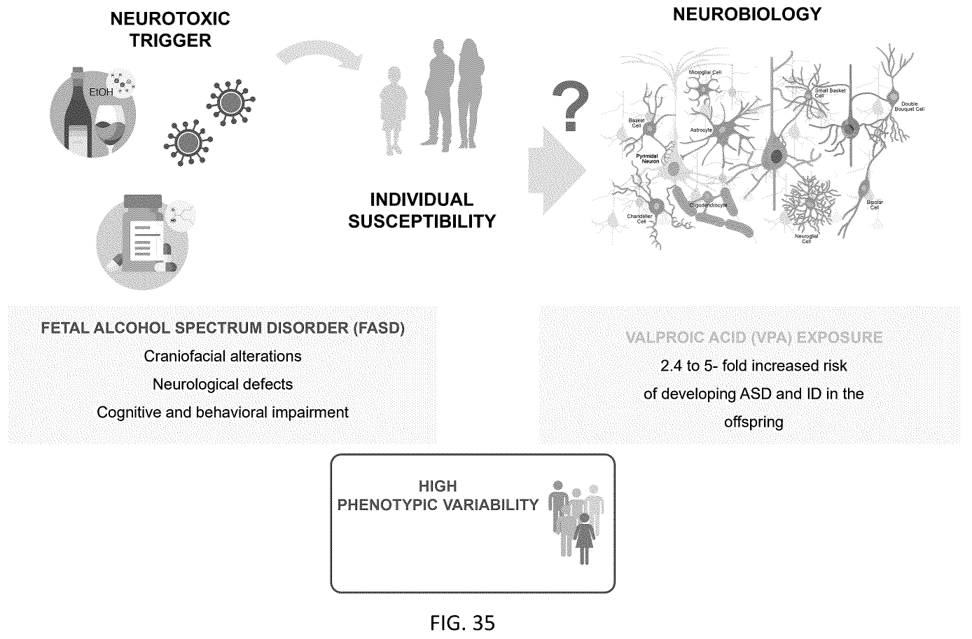

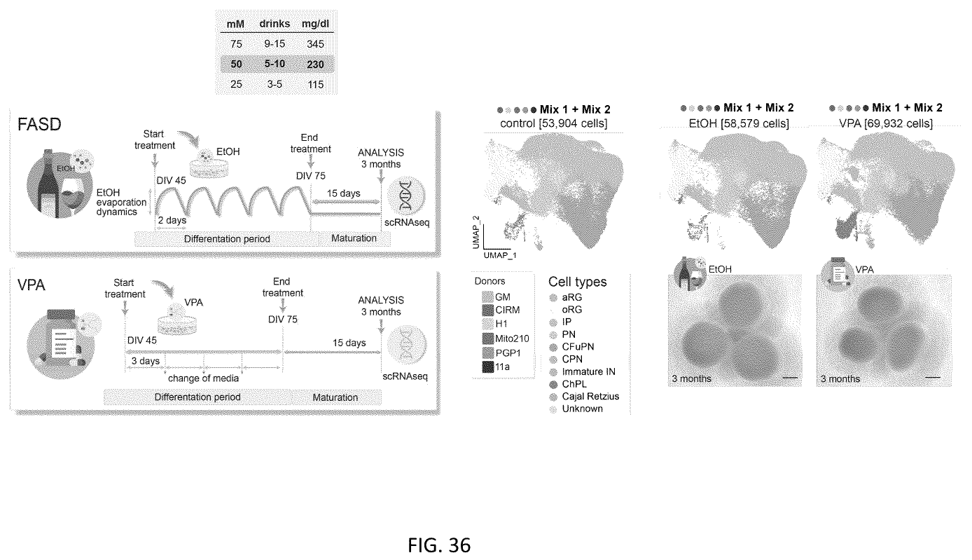

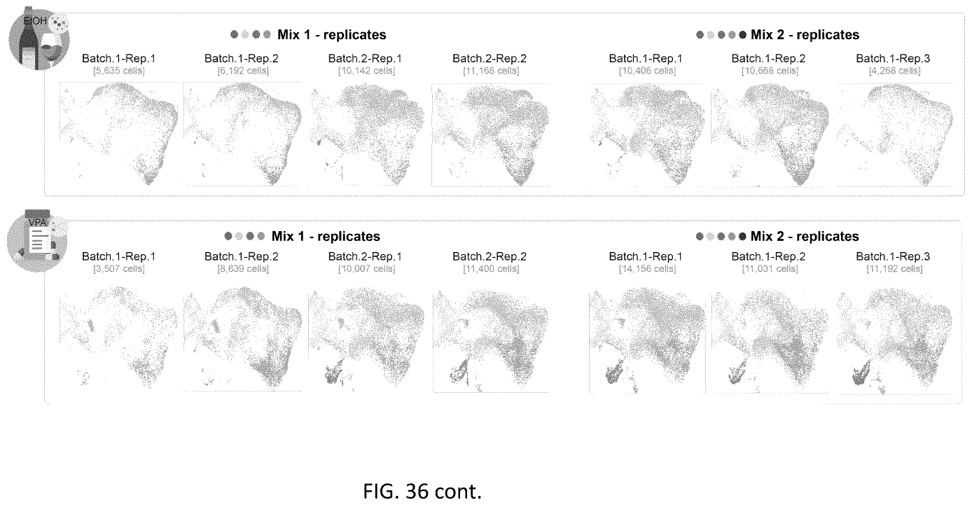

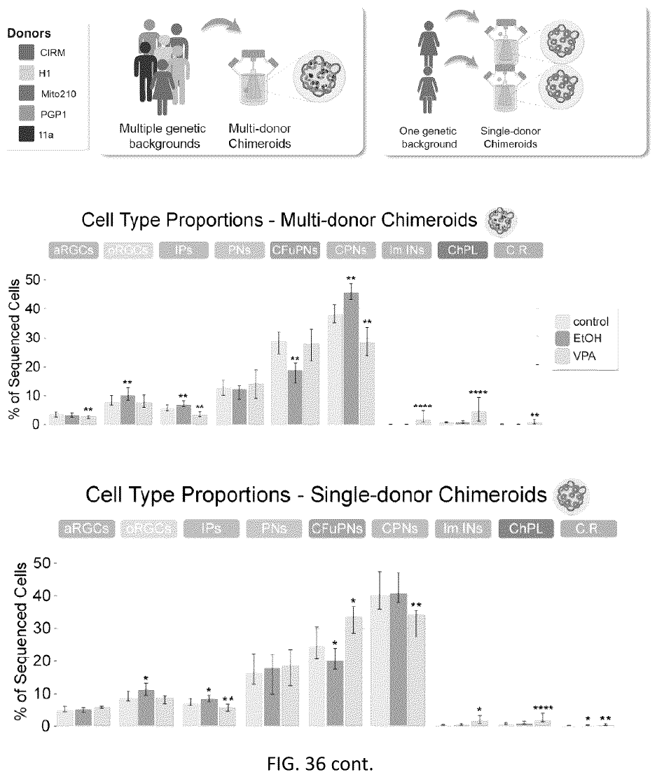

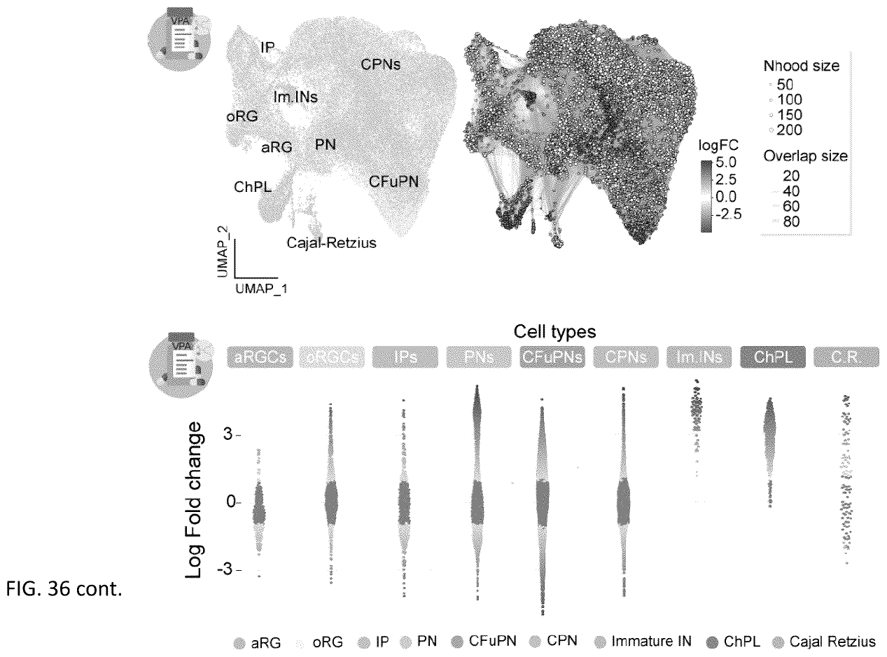

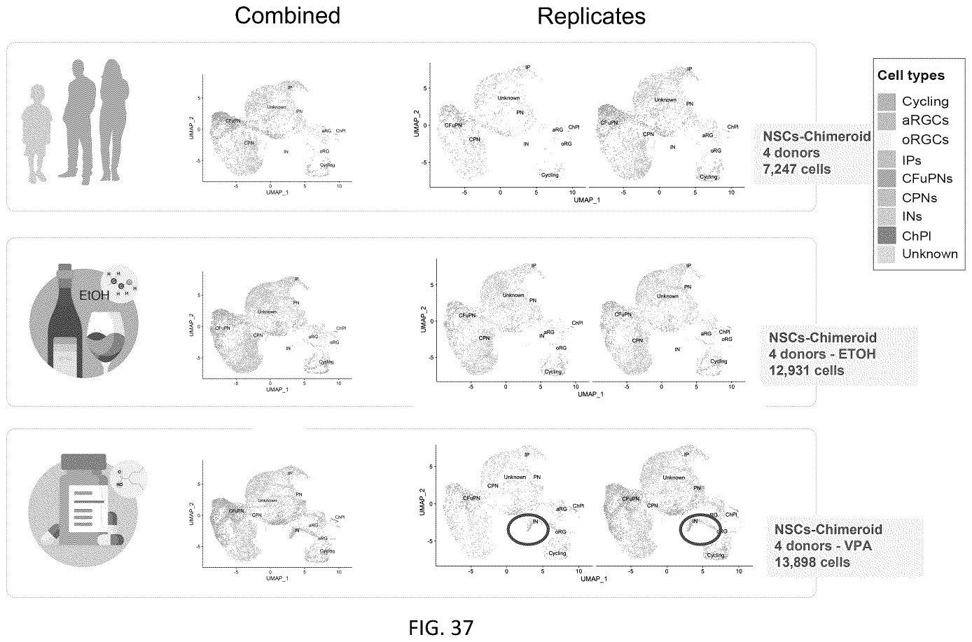

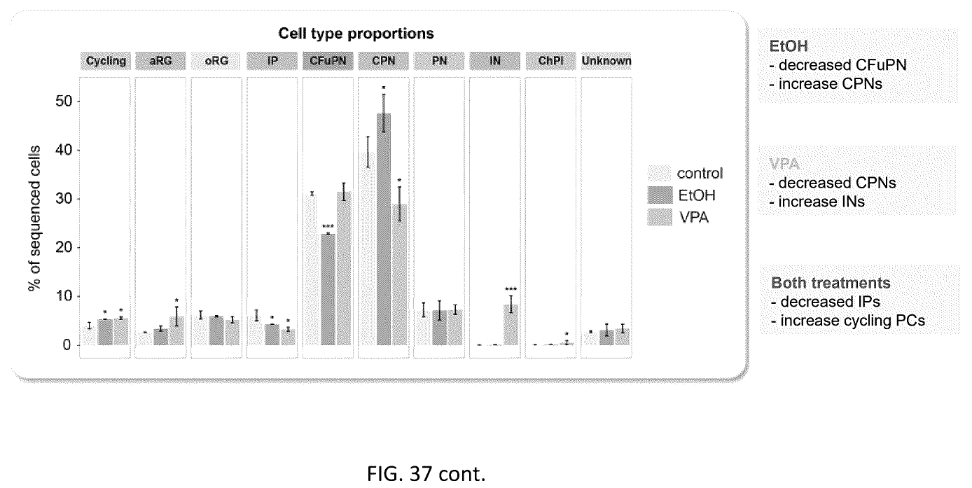

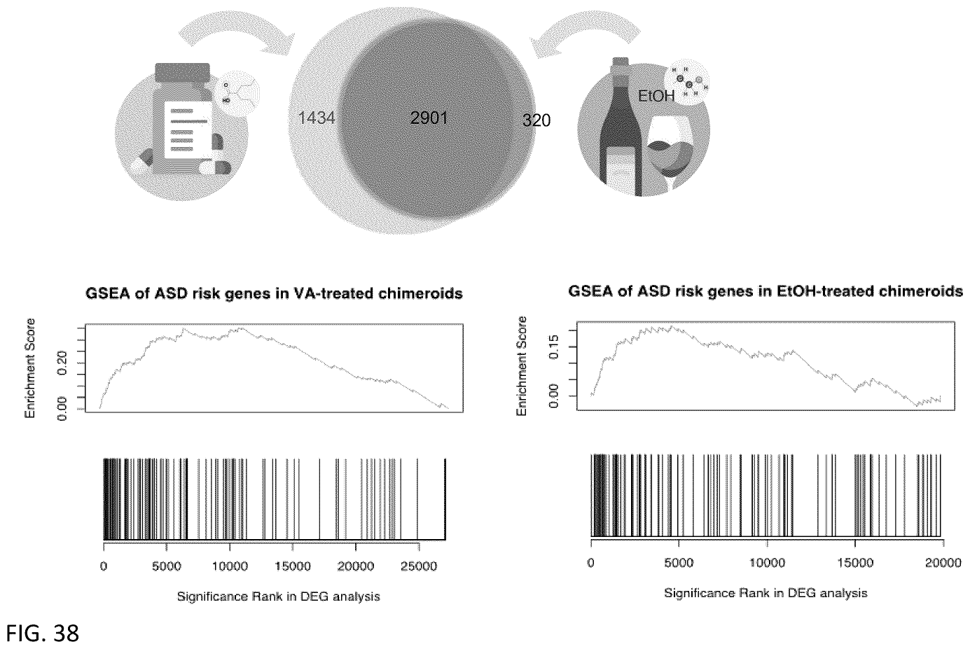

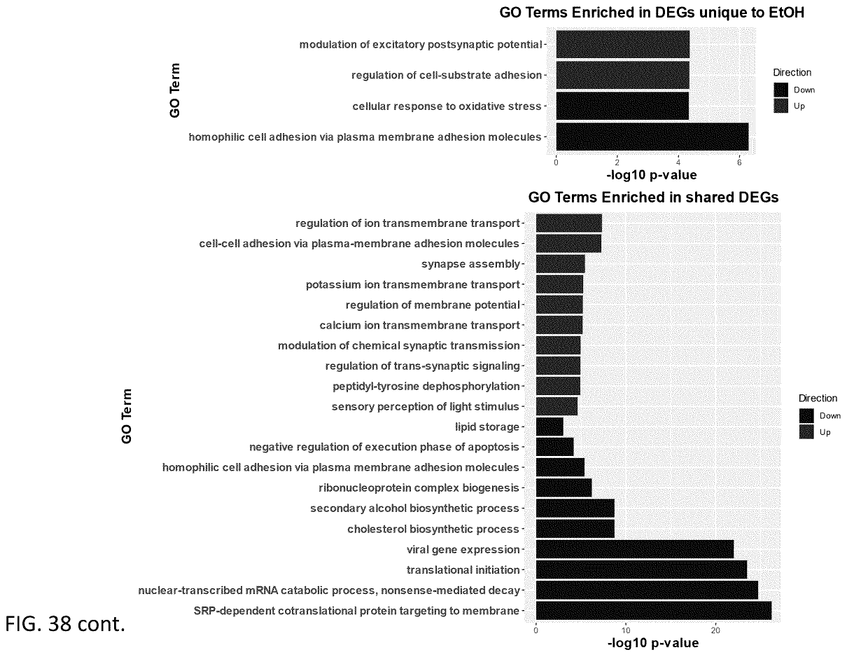

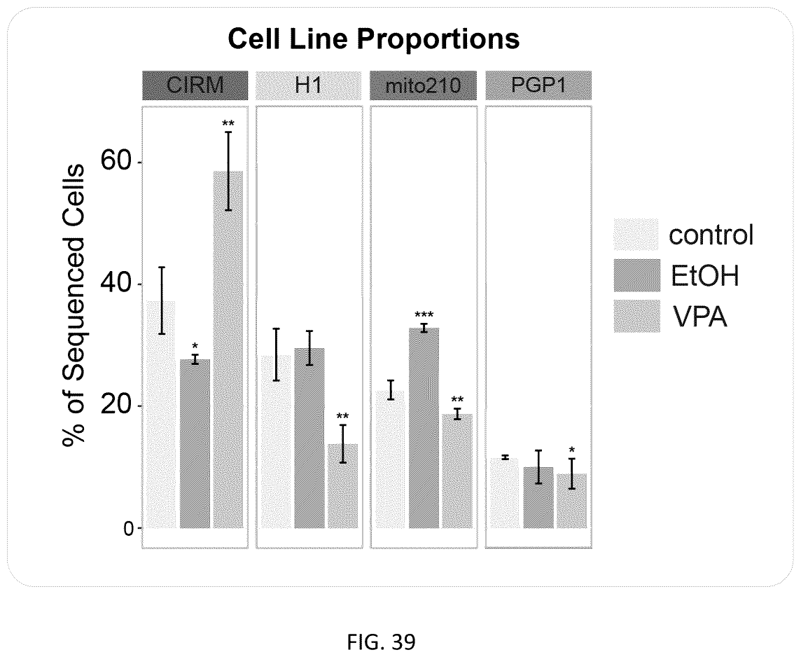

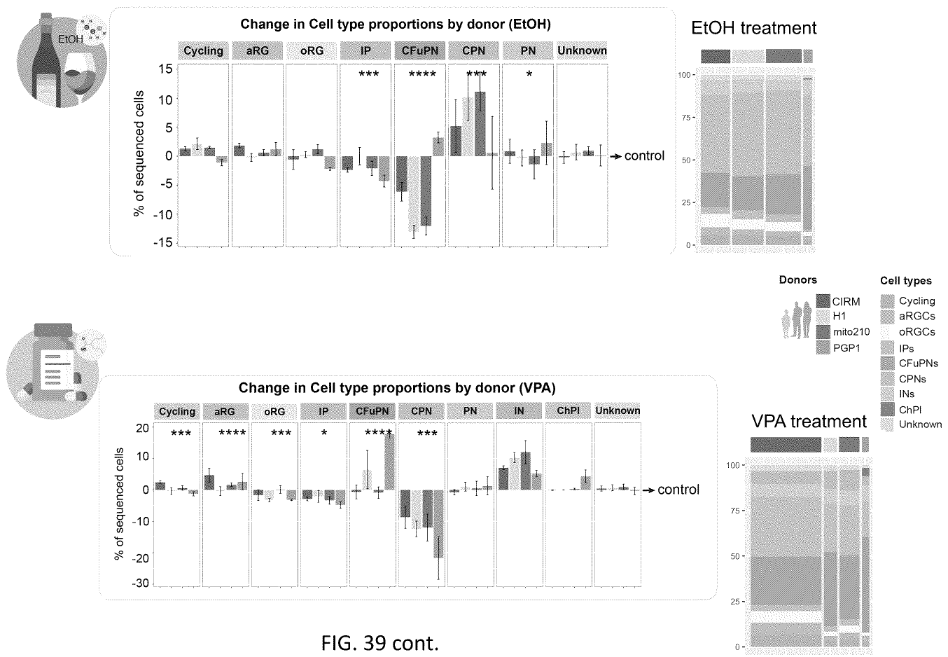

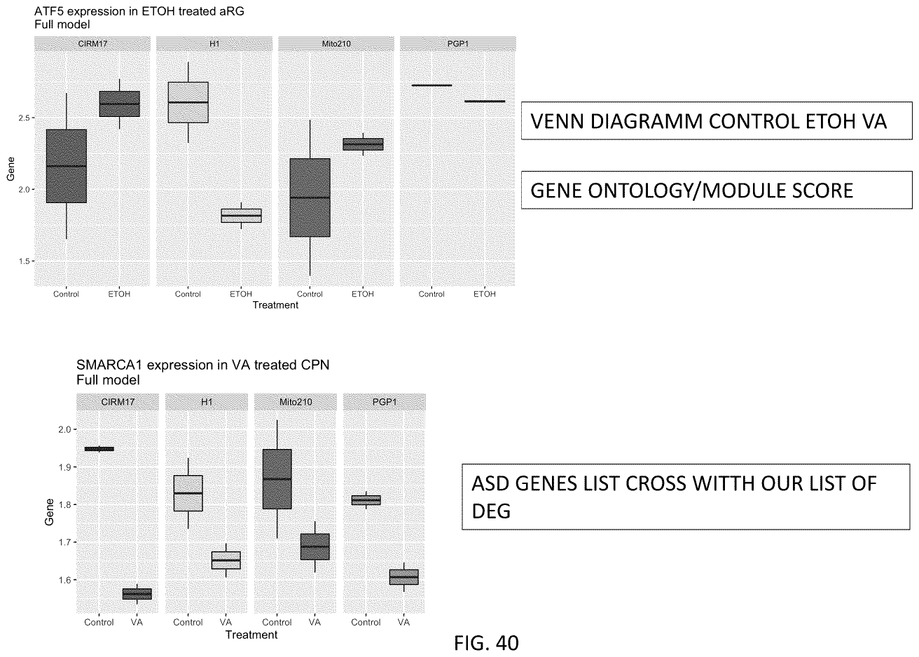

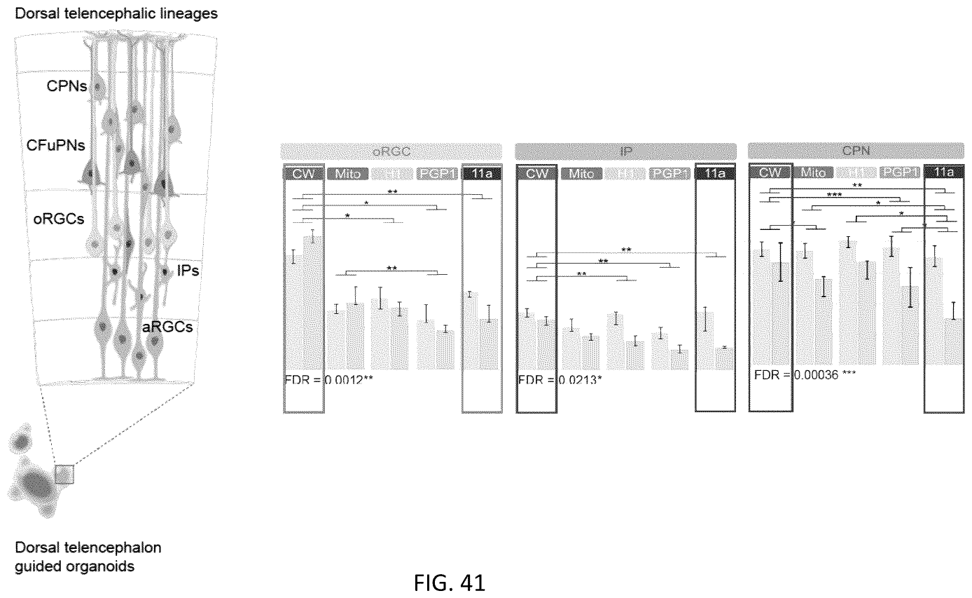

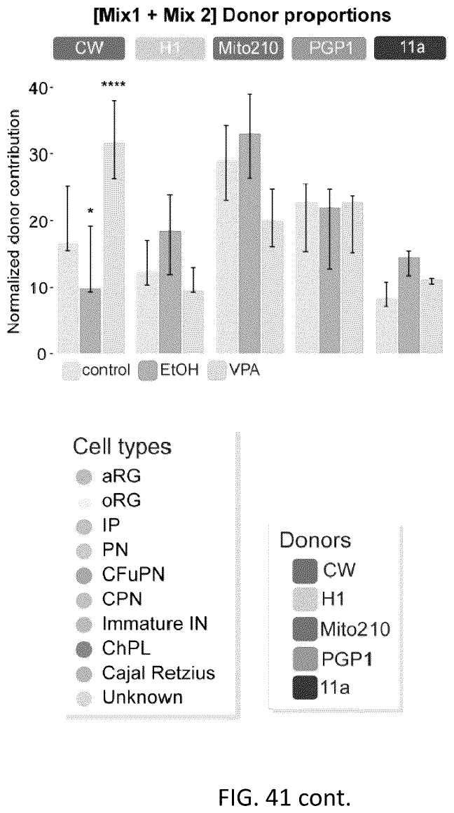

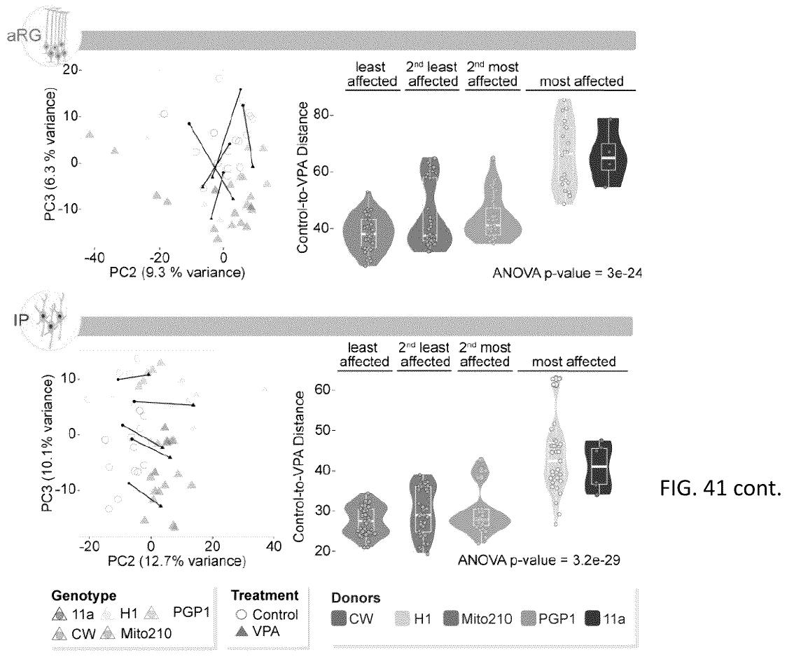

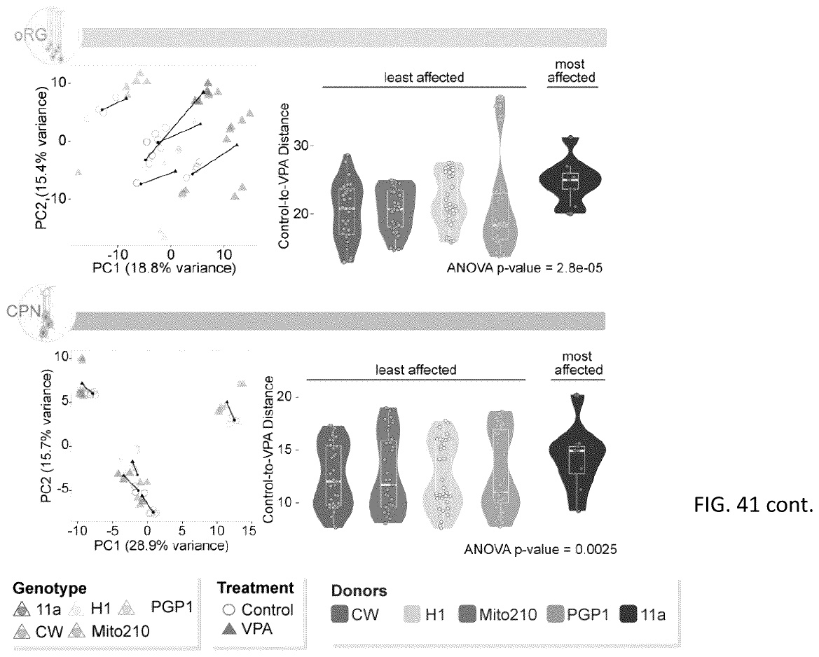

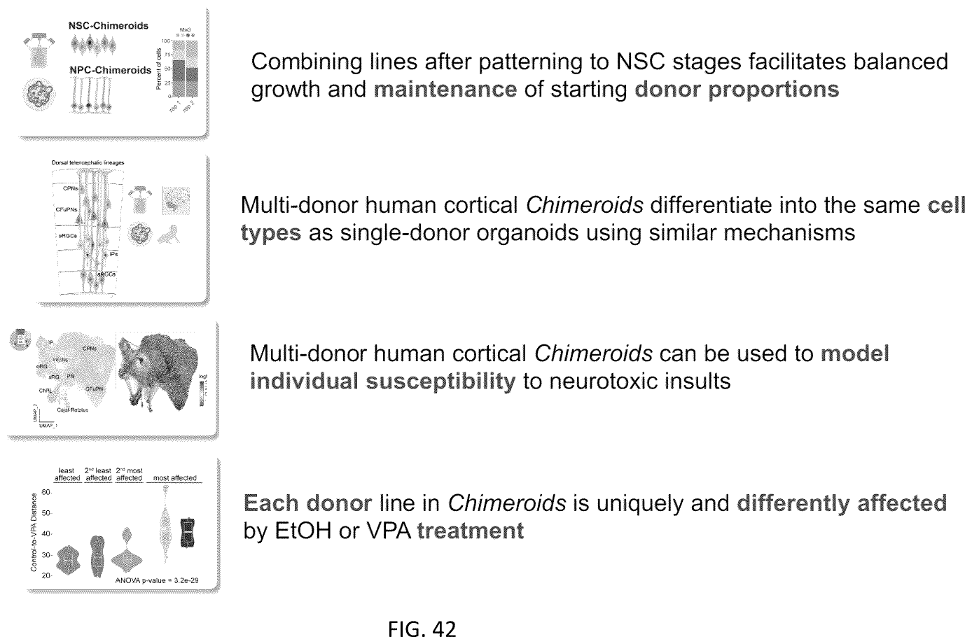

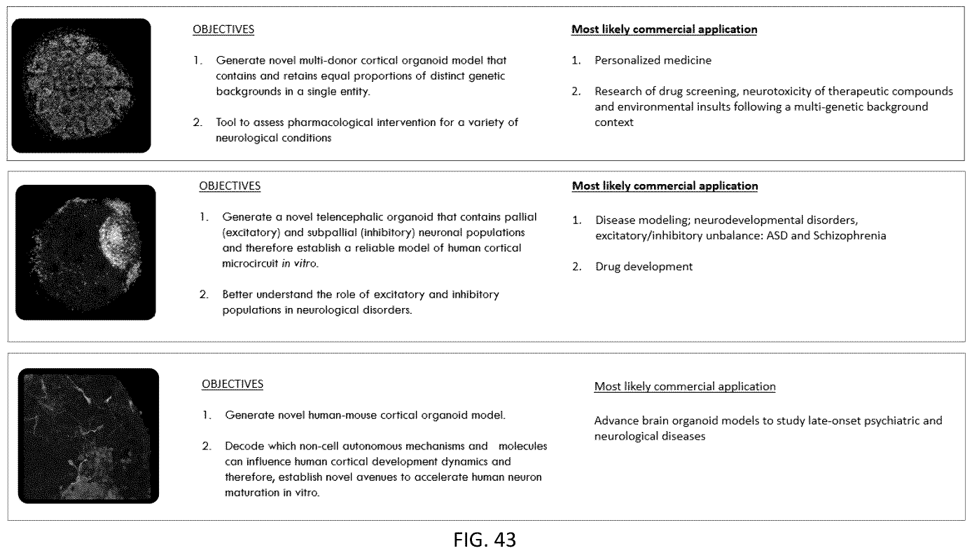

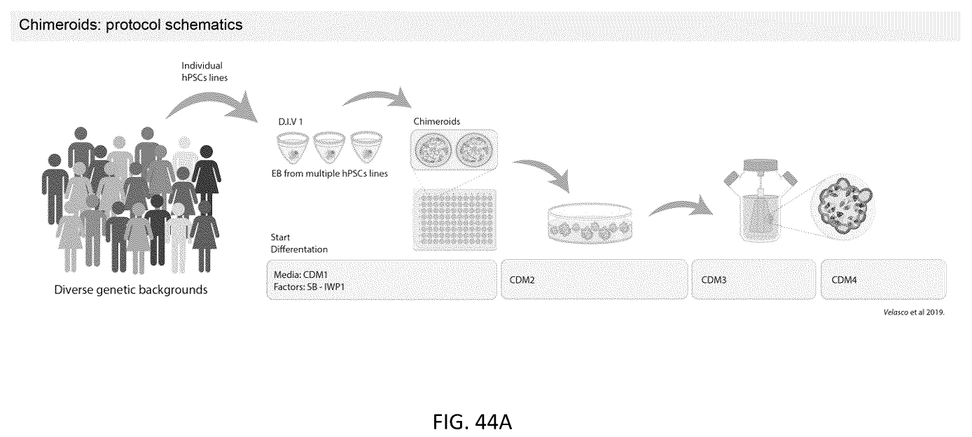

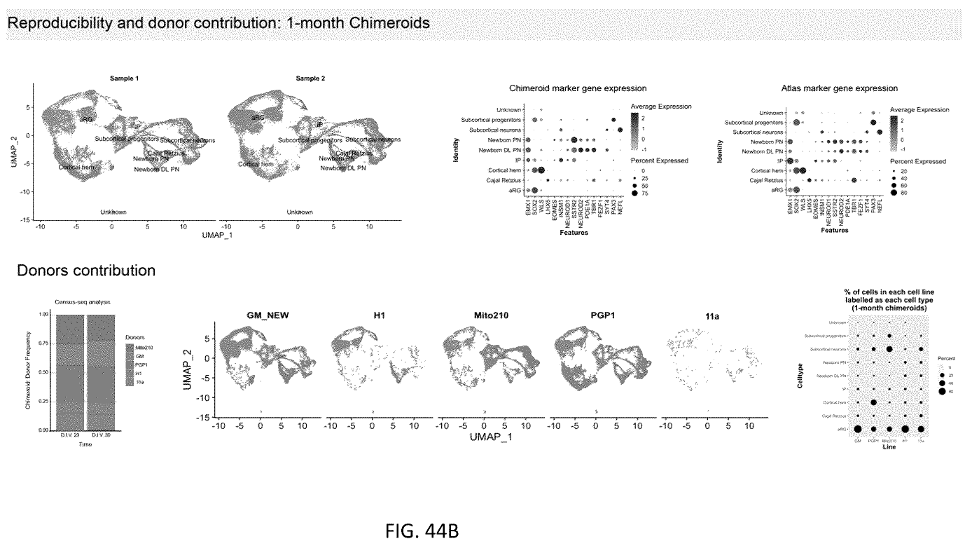

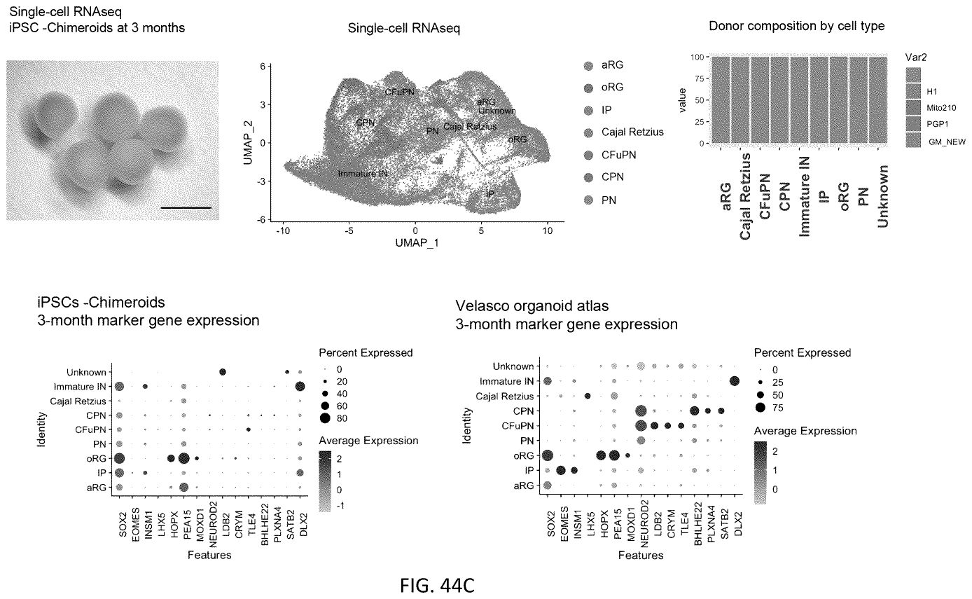

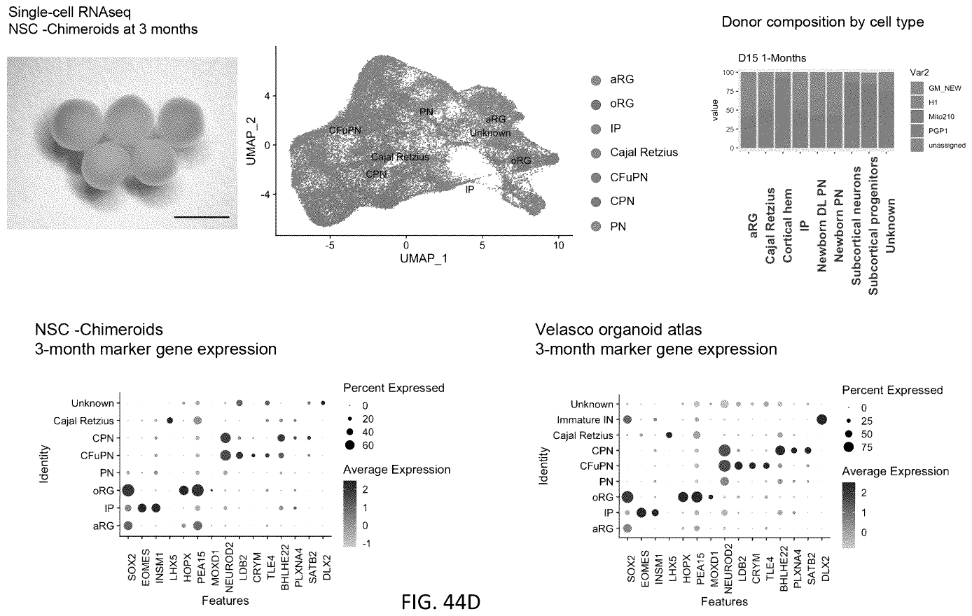

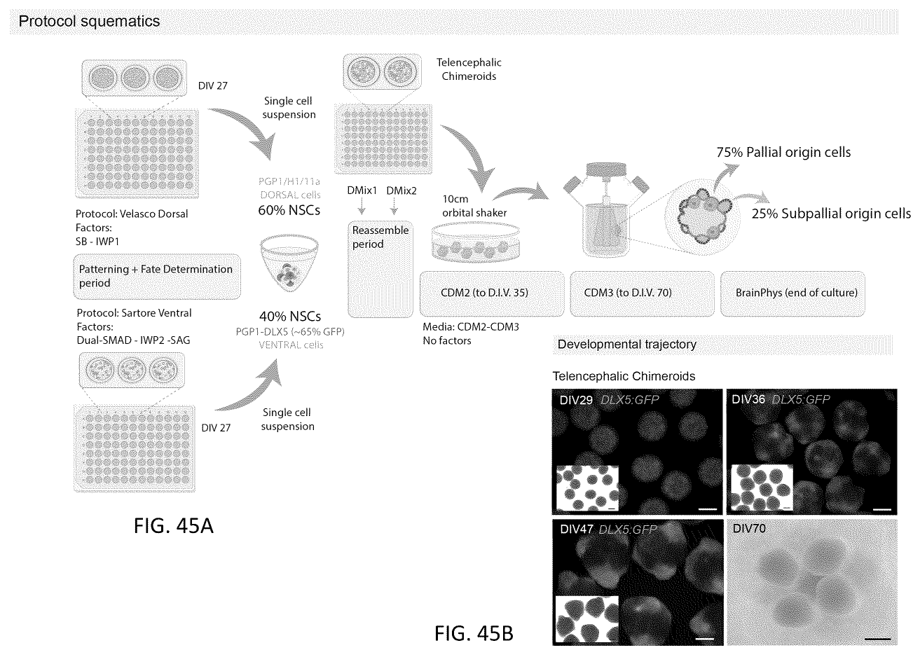

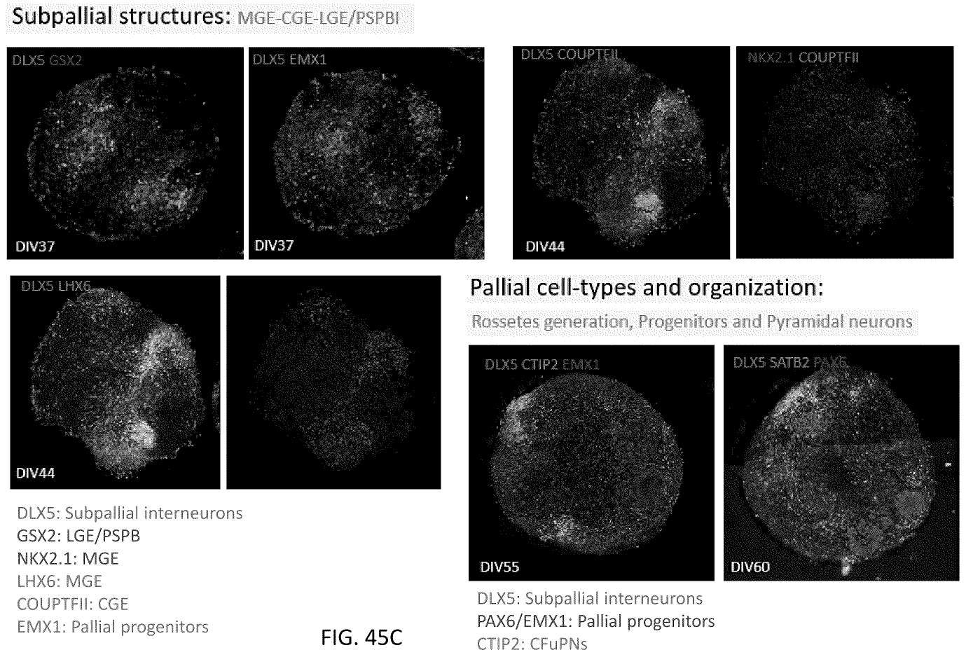

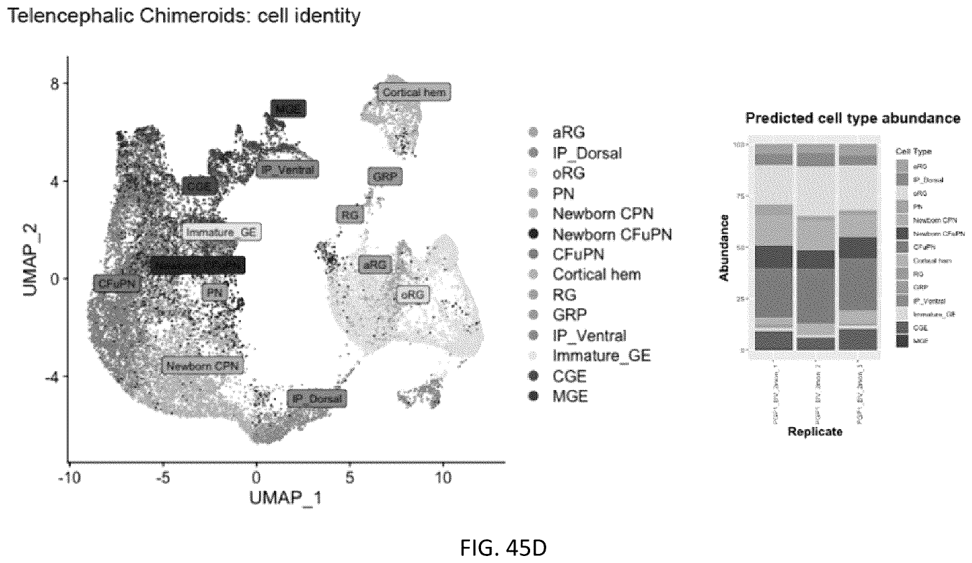

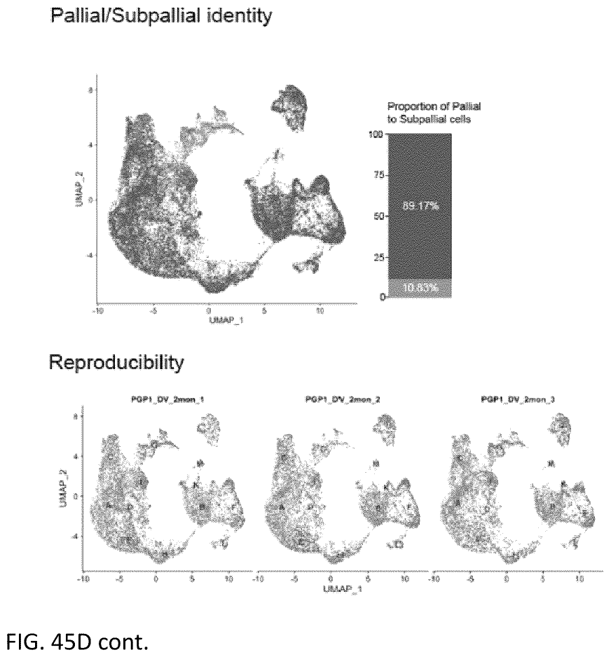

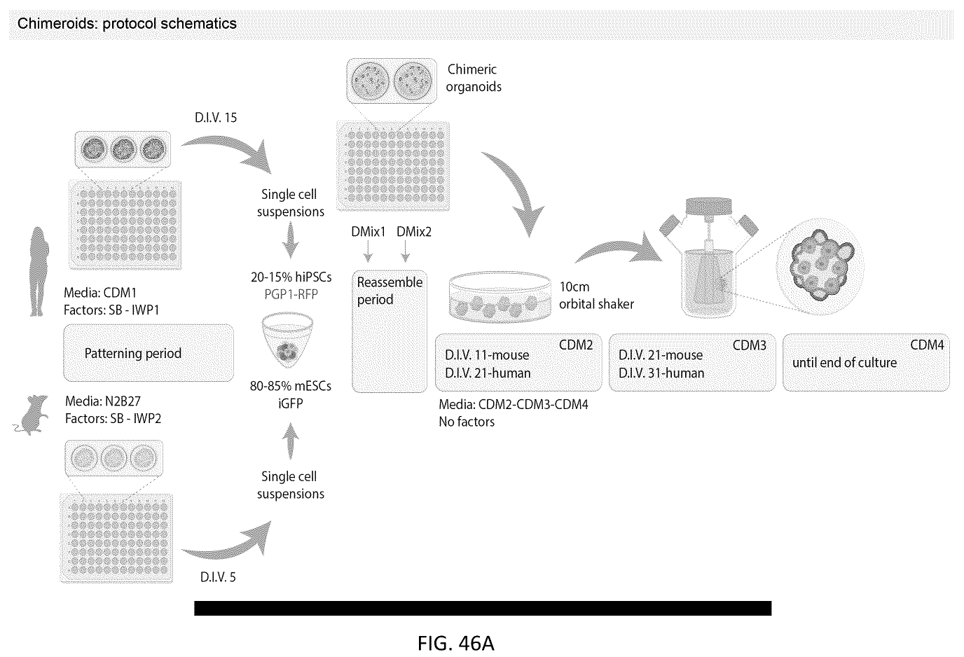

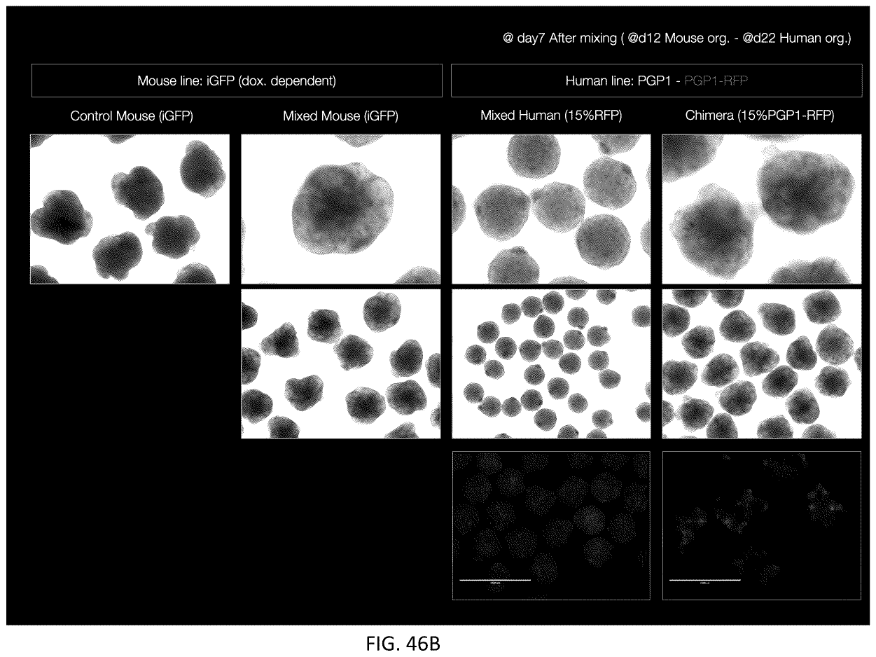

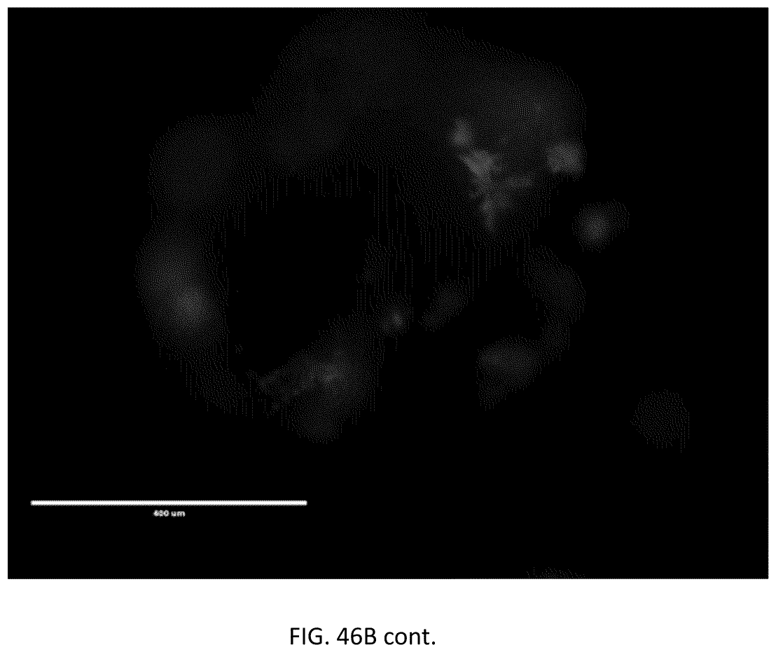

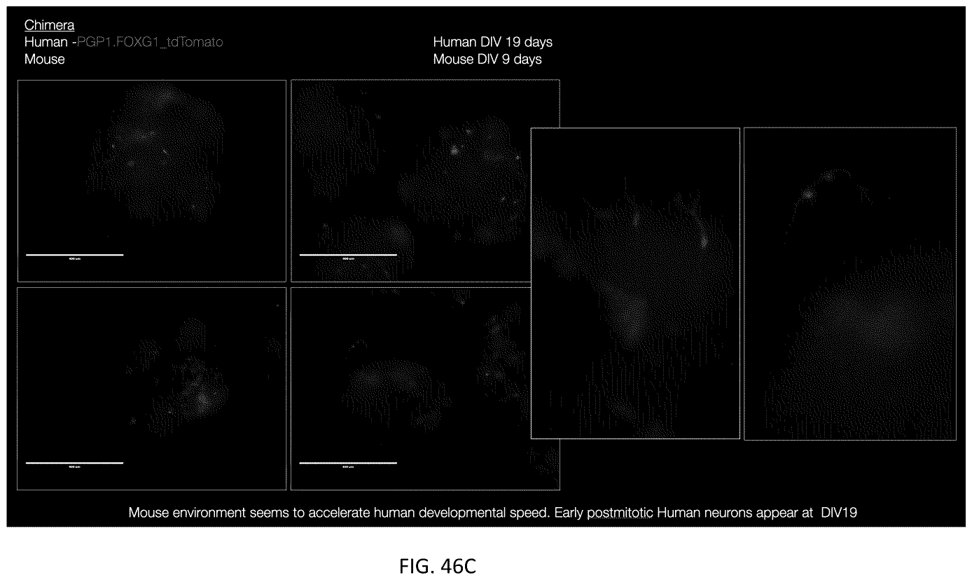

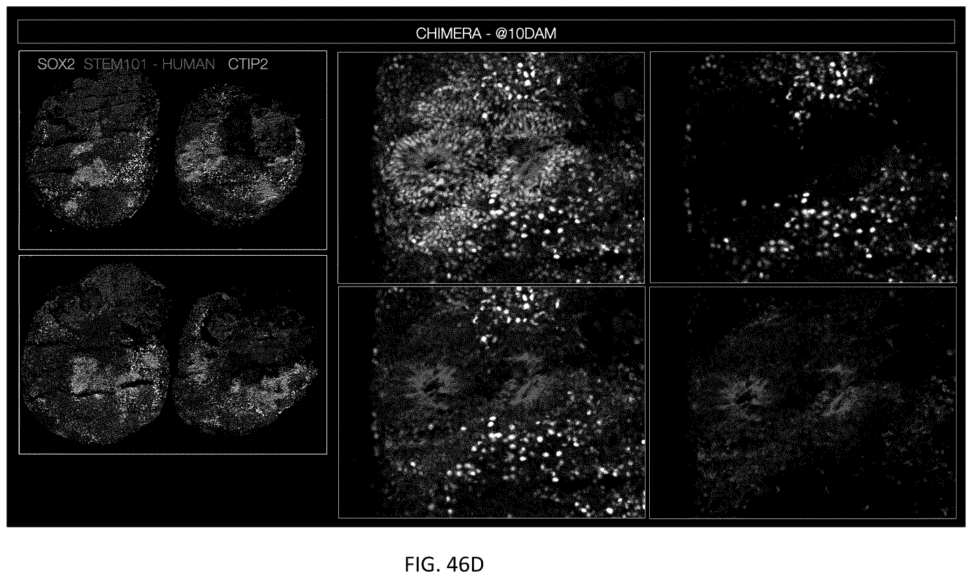

While control Chimeroids displayed the cellular composition previously observed in the single-donor organoid model at this age (FIG. 4B) (Uzquiano et al., Cell, 2022; Velasco et al., Nature, 2019), each treatment group showed distinct and significant alterations in cell-type composition (FIGS. 4F-4G). VPA treatment caused a dramatic increase in the proportion of immature GABAergic interneuron (FDR=9.4*10⁻⁷, negative binomial mixed-effects (NBME) model) and choroid plexus cells (FDR=3.0*10⁻⁶), even when accounting for batch, mix, and donor (Methods) (FIG. 4F, FIG. 13), consistent with previous reports on VPA-treated organoid models (Meng et al., Transl Psychiatry, 2022). Interestingly, previous results from the group identified accelerated interneuron development as one of the convergent phenotypes of haploinsufficient loss-of-function mutations in different ASD risk genes (Paulsen et al., Nature, 2022), suggesting that this cell population may be particularly susceptible to neurodevelopmental risk factors. Other neuronal clusters also showed treatment-specific shifts in proportion: CPN proportions were increased by EtOH treatment (FDR=0.001, NBME model) and decreased in VPA-treated Chimeroids (FDR=0.01), and IP proportions were decreased by both treatments (FDR=0.0042 for VPA and 0.0038 for EtOH) (FIG. 4F).

Because statistical tests for change in proportions of individual cell types do not account for the interdependence between such changes (which must sum to zero), the inventors confirmed these observations and assessed the effects of EtOH and VPA on global expression profiles using Milo (Dann et al., Nat Biotechnol, 2022), a method designed for testing differential cell abundance and expression variance in single-cell data without using cluster identity. Milo corroborated the increase in proportion of immature interneurons and choroid plexus cells, and decrease in proportion of aRG, in VPA-treated Chimeroids, and the decrease in proportion of CFuPN in EtOH-treated Chimeroids (FIGS. 4H-4I). Moreover, Milo highlighted global shifts in the expression landscape without overall change in discrete cell type proportions in oRG, CFuPN, and CPNs in VPA-treated Chimeroids, and in CPN in EtOH-treated Chimeroids (FIG. 4I). The inventors observed similar effects of VPA treatment on cell-type proportions in single-donor Chimeroids (FIG. 4G, FIG. 14), although this analysis had less statistical power due to the smaller dataset size. Overall, VPA caused more expression changes than EtOH treatment, in line with its established role as an epigenetic effector (Göttlicher et al., Embo j, 2001); in VPA-treated Chimeroids, 3,014 out of 8,848 neighborhoods were significantly altered, compared to only 520 out of 7,941 neighborhoods for EtOH-treated Chimeroids.

Overall, these findings demonstrate that VPA and EtOH cause treatment-specific alterations in cell composition and cell-intrinsic expression states that could be detected in multi-donor Chimeroids. Interestingly, VPA exposure, which is correlated with ASD (Bjørk et al., JAMA Neurol, 2022; Christensen et al., JAMA, 2013), induced an increase in the immature GABAergic population, reminiscent of a cellular phenotype induced by haploinsufficiency of several different ASD risk genes in single-donor cortical organoids (Paulsen et al., Nature, 2022). Moreover, similar phenotypic alterations could be detected in single-donor Chimeroids, thus corroborating the use of multi-donor Chimeroids to investigate treatment-specific responses.

### Chimeroids Allow Analysis of Inter-Individual Variation in Response to Neurotoxins

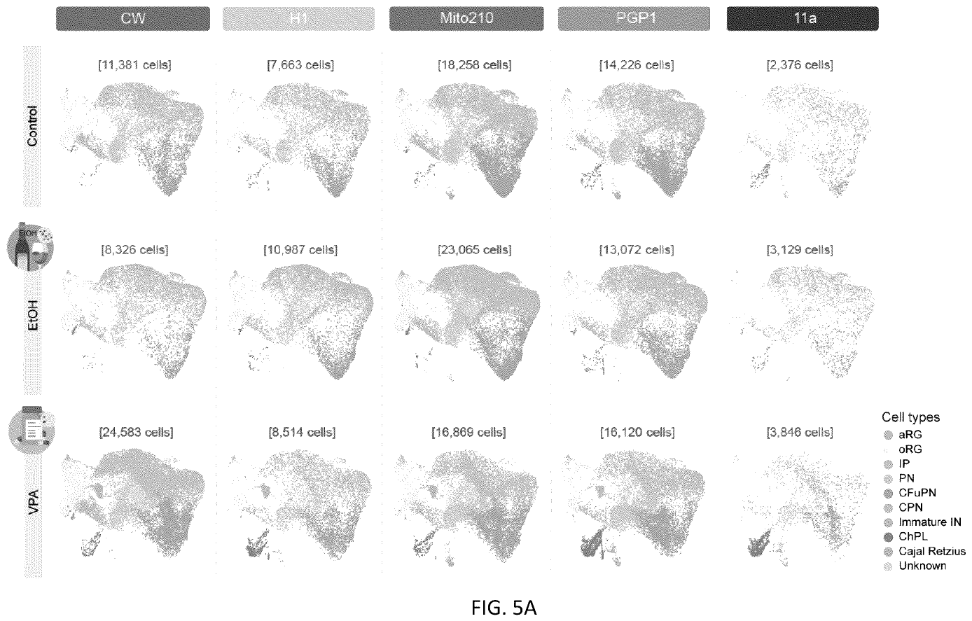

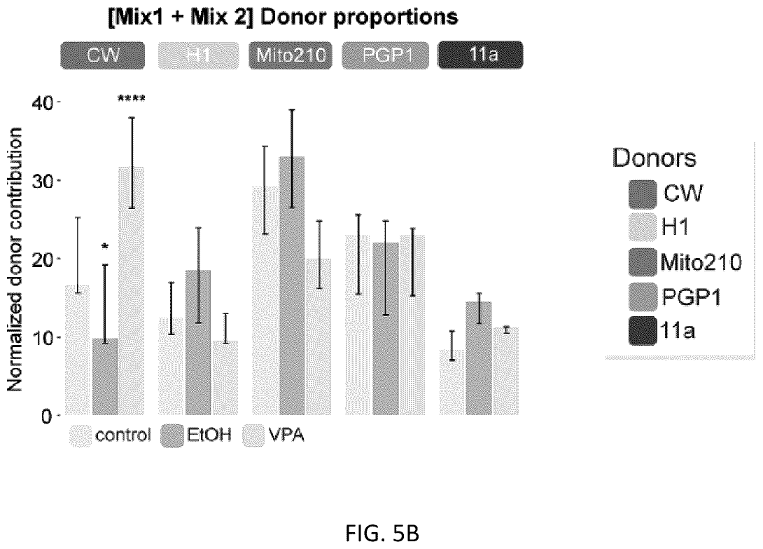

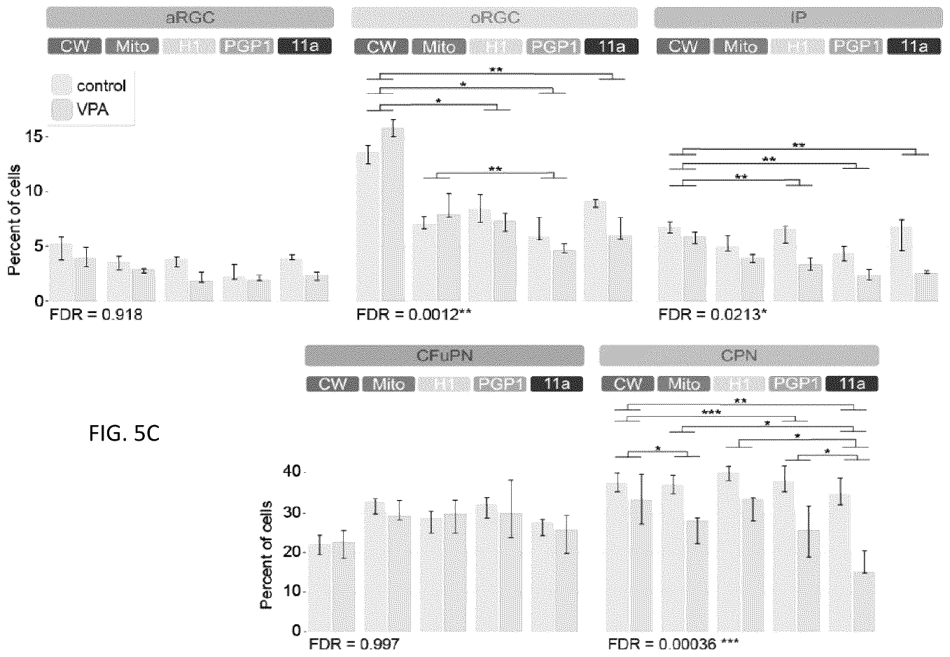

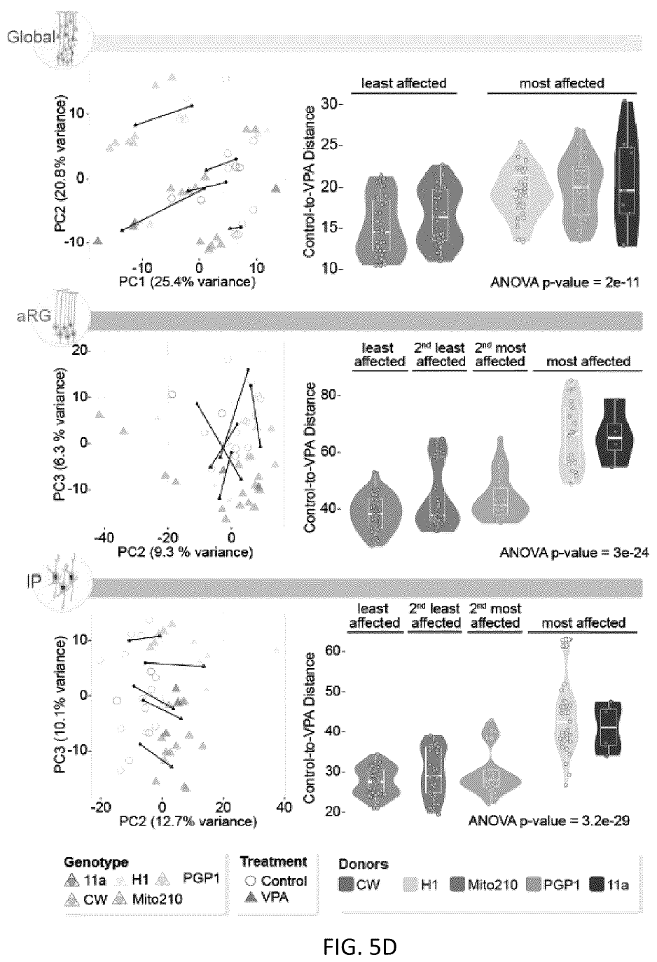

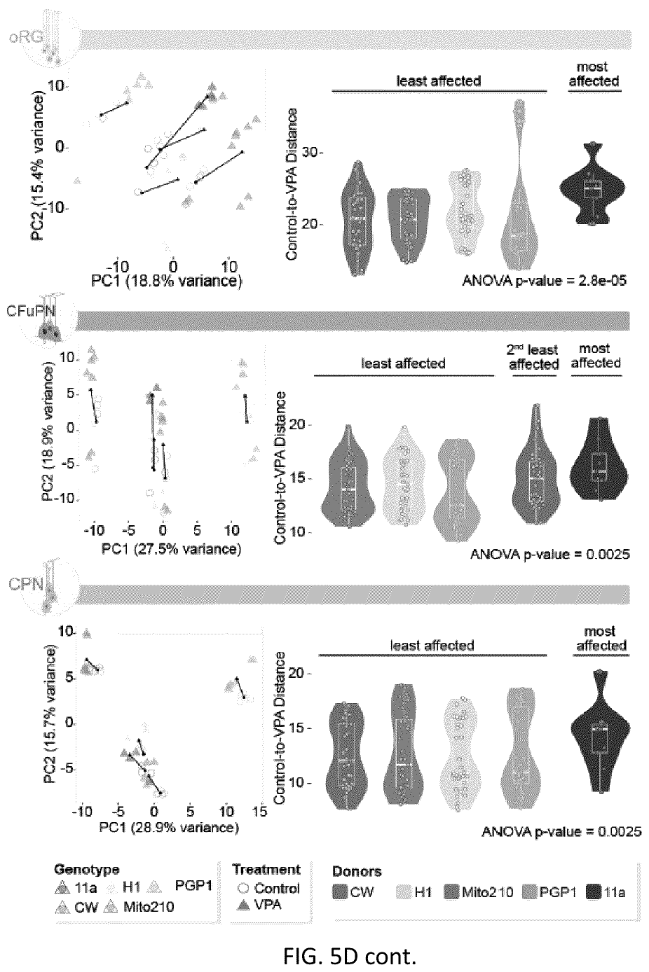

It was next asked whether the inventors could detect donor-specific responses to EtOH and VPA treatment in Chimeroids. While each donor contributed to all cell types, regardless of perturbation (FIG. 5A), the overall proportion of Chimeroid cells derived from individual donors was differentially affected by VPA and EtOH, across two separate mixes (FIG. 5B). Specifically, the proportion of Chimeroid cells derived from the CW donor line was decreased by EtOH treatment (FDR=0.036, (NBME) model) and increased by VPA (FDR=6.5*10-5), indicating a treatment and donor-specific effect of these two compounds (FIG. 5B).

For a more granular analysis of donor-specific effect of each treatment, the inventors tested whether, VPA treatment also differentially affected the proportion of individual cell types for each donor. The inventors found that either the magnitude or the direction of change in representation induced by VPA was significantly different between donors for IP, ORG, and CPN cells (FIG. 5C).

Finally, the inventors examined whether there were donor-specific differences in cell-intrinsic expression changes in the VPA-treated Chimeroids. To summarize the magnitude of differential response in each donor, the inventors calculated pseudobulk expression profiles for each donor within each Chimeroid, both globally (summing the UMI counts of all cells from a donor in a Chimeroid) and separately for each major cell subset (e.g., summing the UMI counts of aRG from one donor in a Chimeroid). For each set of pseudobulk expression profiles (global, aRG, ORG, IP, CFuPN, and PN), the inventors performed principal component analysis (PCA) to identify the major axes of variation, and then compared the Euclidian distance between control and VPA-treated Chimeroids for each donor, calculated on all principal components (Methods). The CW and Mito210 donors showed less separation between VPA and control than did the other donors in global profiles (FIG. 5D). To examine the impact on intrinsic cell states specifically, the inventors performed this analysis for each affected cell subset separately showing that donors also differed in the degree of expression response to VPA within individual cell types (FIG. 5D). For most cell types, the 11a donor was among the most-affected, while CW was among the least-affected; however, for CFuPN, CW showed the second-strongest effect, indicating that individual cell types within each donor show different degrees of perturbation, a finding consistent with the prior report that the effect of genetic background is cell-type specific and perturbation-specific (Paulsen et al., Nature, 2022).

Altogether, the data show that the multi-donor Chimeroid system allows assaying experimental perturbations across multiple donors while still enabling the detection of individual differences in response. The data highlight the value of Chimeroids for high-throughput investigation of the effect of human genetic variation on brain responses.

### Discussion

Human genetic diversity is an important contributor to clinical variation in disease manifestation, but modeling the contribution of genetic background to disease phenotypes of the human brain has been difficult due to the requirement for human experimental brain models that are also capable of accommodating a large variety of individual donors. Not all human PSC lines perform equally well across all in vitro models, especially organoid systems, due to differences inherent to each line (Wells et al., Cell Stem Cell, 2023), such as biases in fate potential and proliferation linked to differential reprogramming, epigenetic imprinting, or susceptibility to culture conditions.

The demonstration that cells derived from multiple donors can grow together and are capable of balanced differentiation within individual Chimeroids indicates that it is possible to investigate, in a single organoid, the collective development and behavior of a large diversity of cell types of the brain, across many human individuals. Because Chimeroid protocols can be adapted to tolerate even extreme donor-to-donor differences, while still allowing detection of differential response to perturbation, donors can be mixed agnostically to stem cell line of origin. This paves the way for efforts over the coming years aimed at scaling-up the donor pools used in Chimeroids to enable investigation of cells from a very large numbers of individuals.

The Chimeroid approach will facilitate assaying across a variety of donor characteristics; for instance, panels of lines that vary in genetic risk for disease, presence or absence of predisposing factors, or clinical phenotype. Combining highly-multiplexed Chimeroids with increased automation of cell culture systems may enable sufficient scaling for investigation of population-level variation across hundreds or even thousands of individuals.

The demonstration that variation in donor response to perturbations such as neurotoxic compounds can be read at cell-type resolution, suggests that Chimeroids may be also used to stratify patients across treatment response groups. Therapeutics for CNS diseases often perform poorly in clinical trials and typically show efficacy in only a subset of patients. The ability to assay drug response at scale using Chimeroids may enable data-driven stratification of patients into different response groups, and in time would produce matrices of drug response data by cell type and by donors to fuel generative models that may predict drug efficacy before clinical investigation.

### Methods

### Human Pluripotent Stem Cell Culture

All human pluripotent stem cell lines were cultured as previously described (Paulsen et al., Nature, 2022; Uzquiano et al., Cell, 2022; Velasco et al., Nature, 2019). Briefly, MTeSR1 medium (StemCell Technologies), mTeSR+ medium (StemCell Technologies), or StemFlex medium (Gibco), all with 1% of added Penicillin-Streptomycin Solution (Corning), were used for culture of stem cells in cell culture dishes (Falcon) previously coated with 1% Geltrex (Gibco), at 37° C. in 5% CO2. All hPS cell lines were maintained below passage 50, tested negative for Mycoplasma (assayed with MycoAlert PLUS Mycoplasma Detection Kit, Lonza) and were karyotypically normal (G-banded karyotype test performed by WiCell Research Institute).

### Characterization of the PSC Lines

The psychiatric control Mito210 male iPS cell line was provided by B. Cohen (McLean Hospital); the PGP1 male iPS cell line (Church, Mol Syst Biol, 2005) was provided by G. Church (Harvard University); the GM08330 male iPS cell line (Sheridan et al., PLOS One, 2011) (also known as GM8330-8 (Sugathan et al., Proc Natl Acad Sci USA, 2014)) was provided by M. Talkowski (MGH) and was originally derived from fibroblasts obtained from the Coriell Institute for Medical Research (Sugathan et al., Proc Natl Acad Sci USA, 2014). The female CW50037 iPSC line was from the California Institute for Regenerative Medicine (CIRM) iPSC collection. The H1 male hESC line (also known as WA01) (Thomson et al., Science, 1998) was purchased from WiCell; and the 11a male iPS cell line (Boulting et al., Nat Biotechnol, 2011) was obtained from the Harvard Stem Cell Institute.

All lines were authenticated as follows: The HUES66 hESC line was authenticated by short tandem repeat (STR) analysis (performed by GlobalStem in 2008), as was the PGP1 iPSC line (performed by TRIPath in 2018) (Uzquiano et al., Cell, 2022). The Mito210 iPSC line was authenticated with genotyping analysis (Fluidigm FPV5 chip) performed by the Broad Institute Genomics Platform (Uzquiano et al., Cell, 2022). The H1 and GM08330 lines were authenticated by STR analysis (performed by WiCell in 2021). The GM08330 parental line has a previously-reported interstitial duplication in the long (q) arm of chromosome 20 (Paulsen et al., Nature, 2022); all other lines were karyotypically normal. All experiments involving human cells were approved by the Harvard University IRB and ESCRO committees.

### Cortical Organoid Differentiation

Dorsally patterned forebrain organoids were generated according to (Velasco et al., Nature, 2019), with some adaptions to the protocol as previously described (Paulsen et al., Nature, 2022; Uzquiano et al., Cell, 2022). Briefly, on day 0, feeder-free cultured human iPSCs or ESCs, 75-85% confluent, were enzymatically dissociated to single cells with Accutase (Gibco), and 9,000 cells per well were reaggregated in ultra-low cell-adhesion 96-well plates with V-bottomed conical wells (sBio PrimeSurface plate; Sumitomo Bakelite) in the same pluripotent cells medium in which they were previously maintained. At day 1, 80 μl of media was replaced with Cortical Differentiation Medium (CDM) I, containing Glasgow-MEM (Gibco), 20% Knockout Serum Replacement (Gibco), 0.1 mM Minimum Essential Medium non-essential amino acids (MEM-NEAA) (Gibco), 1 mM pyruvate (Gibco), 0.1 mM 2-mercaptoethanol (Gibco), with 1% of Penicillin-Streptomycin Solution (Corning). From day 0 to day 6, ROCK inhibitor Y-27632 (Millipore) was added to the medium at a final concentration of 20 μM. Patterning small molecules, Wnt inhibitor IWR1 (Calbiochem) and TGFβ inhibitor SB431542 (Stem Cell Technologies), were added from day 0 to day 15-18, at a concentration of 3 μM and 5 μM, respectively.

From day 15-18, patterned EBs were cultured under orbital agitation (Thermofisher CO₂ resistant orbital shaker) in ultra-low attachment culture dishes (Corning) in CDM II medium, containing DMEM/F12 medium (Gibco), 2 mM Glutamax (Gibco), 1% N2 (Gibco), 1% Chemically Defined Lipid Concentrate (Gibco), 0.25 μg/mL Fungizone (Gibco), with 1% of Penicillin-Streptomycin Solution (Corning). On day 35, cell aggregates were transferred to spinner-flask bioreactors (Corning) and maintained in CDM III (CDM II supplemented with 10% Fetal Bovine Serum (FBS) (GE-Healthcare), 5 μg/mL heparin (Sigma) and 1% Matrigel (Corning)). From day 70, organoids were cultured in CDM IV (CDM III supplemented with B27 supplement (Gibco) and 2% Matrigel).

### Chimeroid Aggregation Step

The inventors tested 9,000, 12,000, and 15,000 cells for the aggregation step during NSC-Chimeroid generation. Chimeroids derived from the aggregation of less than 15,000 cells remained very small and of uneven size, and immunohistochemistry showed scarce differentiation towards a cortical fate. The inventors therefore increased the seeding cell numbers per well to 15,000-18,000 (data not shown), and 20,000 cells. The inventors observed that 15,000, 18,000, and 20,000 cells per well successfully generated Chimeroids that followed the proper developmental trajectories of the established organoid protocol (Velasco et al., Nature, 2019) (FIG. 7).

For the NPC-Chimeroids, the inventors increased the cell number to 50,000 because a subset of the cells at this stage do not contribute to the further development of the organoid, and ~20% of organoid cells at this stage are postmitotic (Uzquiano et al., Cell, 2022); these postmitotic cells do not have the capability to re-enter the cell cycle and are unlikely to contribute to Chimeroid development after aggregation.

### Multi-Donor Cortical Chimeroids Generation

Chimeroid generation is based on a reaggregation process using putative NSCs or NPCs originating from dorsally-patterned forebrain organoids (cortical organoids) dissociated at distinct developmental stages; NSC-Chimeroids and NPCs-Chimeroids are derived from DIV15-18 and DIV23-25 cortical organoids, respectively.

At the desired developmental stage, pools of 15-30 cortical organoids from each donor were enzymatically dissociated into a single-cell suspension using the Worthington Papain Dissociation System kit (Worthington Biochemical). This protocol was based on (Velasco et al., Nature, 2019), with adaptations to generate Chimeroids. Specifically, cortical organoids were inspected by bright-field microscopy to confirm proper morphology, and collected into low-binding 1.5 mL tubes (Eppendorf). Excess media was removed before enzymatic dissociation. Warmed Papain solution (37° C., 500 μl) was added to the tube, and organoids were triturated 10 times using a P1000 pipette and then incubated for 25 min at 37° C. with agitation on an orbital shaker at 90 RPM. Tissue pieces were then further triturated with a P1000, followed by an additional incubation of 5 min at 37° C. The resulting solution was moved to 15 mL tubes (Falcon) containing 1 mL of Earle's balanced salt solution and 600 μl of ovomucoid inhibitor solution, then centrifuged 5 min at 300 g. The supernatant was discarded, and dissociated cells were resuspended in 1 mL of CDM1 containing 20 μM of ROCK inhibitor Y27632 (Millipore). To remove aggregates and produce a single-cell solution, dissociated cells were filtered using a 35 μm cell strainer tube (Corning), and then counted using a Countess II automated hematocytometer (Thermo Fisher Scientific). Cell suspensions derived from different donors were mixed at equal ratios, and 18,000-20,000 cells (adjusted to allow at least 1000 cells per donor) per well were reaggregated in ultra-low cell-adhesion 96-well plates with V-bottomed conical wells (sBio PrimeSurface plate; Sumitomo Bakelite). The following day, the media was replaced with 100 μl of CDM2 per well. Two days after mixing, reaggregated EB's were passaged in ultra-low attachment culture dishes (Corning), and cultured under orbital agitation (Thermofisher CO₂ resistant orbital shaker). The subsequent media changes of the Chimeroids followed the published protocol (Velasco et al., Nature, 2019) changing to CDM3 media at DIV35, and then to CDM4 at DIV70.

### EtOH and VPA Treatment

At DIV45, multidonor and single-donor Chimeroids were treated with either EtOH or VPA. EtOH (E7023-6, Millipore-Sigma) was administered directly to the culture media every two days at a concentration of 50 μM and the plate was sealed with Parafilm M (Bemis) for 24 hours after administration. VPA (P4543-25G, Millipore-Sigma) was added to the culture media at a concentration of 0.7 mM and the media was changed twice per week. Both treatments were stopped at DIV75, and organoids were analyzed at 3 mo.

### Fixation and Processing of Samples for Cyrosectioning

Chimeroids were fixed in 4% paraformaldehyde (PFA) (Electron Microscopy Services) overnight (O/N) in a 12-well plate (Falcon) at 4° C., washed with 1× phosphate buffered saline (PBS) (Gibco) 3 times, and cryoprotected in a 30% sucrose solution (Sigma) in PBS (O/N) at 4° C.

Samples were then embedded in bovine gelatin. Gelatin solution containing 10% of bovine gelatin (Sigma) and 7.5% of sucrose (Sigma) was prewarmed at 37° C. for 15 min. The 30% sucrose solution was removed from the samples and exchanged for the prewarmed gelatin, and the samples were incubated at 37° C. for 15 min. Meanwhile, plastic molds were coated with a 2 mm layer of warm gelatin solution and left to polymerize at room temperature (RT). Chimeroids were transferred to the pretreated plastic molds, and 1 mL of warm gelatin solution was added to cover the Chimeroids. Following polymerization at RT for 3 min, Chimeroids were pre-chilled at 4° C. for 15-20 min. Finally, the molds were frozen in a cold bath containing 100% EtOH and dry ice for 2-3 min, and stored at −80° C. indefinitely.

### Immunohistochemistry

For immunohistochemistry, 14 to 20 μm thick sections were cut using a cryostat (Leica). Cryosections were stabilized at RT for 5 min and blocked with 10% donkey serum (Sigma)+0.3% Triton X-100 (Sigma) in PBS. Primary antibodies were diluted in the blocking solution and incubated O/N. After 4 washes with PBS, cryosections were incubated at RT with secondary antibodies diluted in PBS (1:1000) for 1 hour at room temperature, washed 4× with PBS, and stained with DAPI (1:10,000 in PBS+0.1% Tween-20) for 15 minutes to visualize cell nuclei.

### Microscopy

Immunofluorescence images were acquired with a Zeiss Axio Imager.Z2 and Lionheart™ FX Automated Microscope (BioTek Instruments). Images were analyzed and processed with the Gen5 (BioTek Instruments) and Zen Blue (Zeiss) image processing software, followed by ImageJ (Fiji) (Schindelin et al., Nat Methods, 2012).

Dissociation of Brain Organoids and scRNA-Seq

Organoids were dissociated as previously described (Velasco et al., Nature, 2019), with the exception of the 1-mo organoids, which were dissociated using low-binding 1.5 mL tubes (Eppendorf) as described in the Chimeroid generation section. Dissociated cells were resuspended in 100 μl of PBS, filtered with a 35 μm cell strainer tube (Corning) to avoid aggregates, and counted with a Cellometer K2 instrument (Nexcelom) or a Countess II automated hematocytometer (Thermo Fisher Scientific). Single-cell suspensions were loaded into the Chromium Next GEM Chip G (10× Genomics, 1000120) and run with the Chromium Controller to generate single-cell GEMs. scRNA-seq libraries were prepared with the Chromium Single Cell 3′ Library and Gel Bead Kit v3.1 (10× Genomics, 1000268). When Chimeroids are composed of cells from multiple genetic backgrounds, it is possible to use that genetic variation to identify doublets containing cells from two different cell lines. This enabled us to tolerate a higher doublet rate and load ~20,000-30,000 cells per channel. The resulting libraries were pooled based on molar concentration and sequenced on a NextSeq 500 or NovaSeq 6000 instrument (Illumina) with 28 bases for read 1, 55 bases for read 2 and 8 bases for index read 1. If necessary, after the first round of sequencing, the inventors re-pooled libraries based on the actual number of cells in each library, and re-sequenced with the goal of producing an equal number of reads per cell for each sample (with a target read depth of 20,000 reads/cell).

Single Cell RNA-Seq data processing

All scRNA-seq data were processed using 10× Genomics Cell Ranger v7.1.0 (Zheng et al., Nat Commun, 2017). Raw BCL files were converted to FASTQ files using the mkfastq command with default parameters. Cellranger's count function was used on the resulting FASTQ files to produce cell-by-gene count matrices for each organoid, with the pre-built GRCh38 human reference genome provided on the 10× Genomics downloads page (available at support.10×genomics.com/single-cell-gene-expression/software/downloads/latest GENCODE v32/Ensembl 98). The expect-cells flag was set manually for each sample, with values ranging from 10,000 to 30,000. All other parameters were set to default. Cellrangers's filtered_feature_bc_matrix was imported into Seurat v4.3.0 (Stuart et al., Cell, 2019) using R version 4.2.2 for downstream analyses.

### Genetic Demultiplexing

For genetic demultiplexing based on scRNA-seq data, demuxlet was applied (Kang et al., Nat Biotechnol, 2018), with default settings, to the alignments produced by Cellranger. Reference variant-call-format (VCF) files generated from whole-genome sequencing of each donor cell line were processed with bcftools (Danecek et al., Gigascience, 2021) to normalize the reference VCFs into biallelic variants, and to remove non-passing and monomorphic variants. Droplets that demuxlet determined to have ambiguous genetic origins, or which were likely to be heterogeneous doublets, were removed with default settings from the Seurat objects before downstream analysis.

In order to determine the proportional abundance of each donor in PSC, NSC, and NPC Chimeroids at different timepoints and from different mixes, Census-seq was applied to low-pass WGS data (Wells, 2023), using the Census-seq function from the Drop-seq software package with default parameters, following the Census-seq computational protocols found at github.com/broadinstitute/Drop-seq, and using the same reference VCF files as mentioned above for each donor.

### Cell Profile Quality Filtering, Normalization, Clustering, Annotation, and Integration

For each Chimeroid, cell profiles with fewer than 200 expressed genes, fewer than 500 UMIs, more than 20,000 UMIs, or greater than 15% mitochondrial RNA, were removed. Seurat's SCTransform function was then run on each organoid's filtered counts matrix, regressing out the percent mitochondrial RNA. Principal component analysis (PCA) was performed on the variable features found by SCTransform, Seurat's FindNeighbors function was used to construct a k-nearest-neighbor graph (k=20) with the top 30 PCs, followed by Louvain clustering using the FindClusters function, with resolution set between 0.2 and 1.0.

Single-cell expression data from multiple Chimeroids were combined into several larger joint datasets based on protocol and treatment as follows: PSC-Chimeroids of Mix 1, Mix 3, and Mix 4 (n=5; 62,242 cells); PSC-Chimeroids of Mix 5 (n=2; 15,612 cells); control, EtOH-treated, and VPA-treated NSC-Chimeroids of Mix 1 and Mix 2 (n=21; 200,019 cells); NSC- and NPC-Chimeroids of Mix 3 and Mix 4 (n=6; 52,771 cells); and control, EtOH-treated, and VPA-treated single-donor NSC-Chimeroids (n=45; 112,166 cells). To visualize scRNA-seq data from multiple organoids in shared UMAP embeddings, raw UMI counts matrixes were merged using Seurat's merge function, then jointly renormalized with SCTransform. PCA was performed on the joint expression matrix, followed by k-nearest-neighbor graph construction and clustering using the same methods as on the individual datasets. When merging all control, VPA-treated, and EtOH-treated Mix 1 and Mix 2 NSC Chimeroids, there was a clear batch effect which resulted in two of the organoids clustering entirely separately from all other organoids. In this case, dataset integration was performed as in Seurat's integration vignette using SCTransform; specifically, using the functions SelectIntegrationFeatures (with the nfeatures parameter set to 3000), PrepSCTIntegration, FindIntegrationAnchors, and IntegrateData. PCA was performed on the integrated dataset, and cell profiles (top 30 PCs) were embedded by Uniform Manifold Approximation and Projection (UMAP).

Within each dataset, each cluster was manually annotated with a cell-type label based on a combination of (1) canonical marker gene expression, (2) examining a list of upregulated genes within each cluster as determined by Seurat's FindMarkers function, and (3) automated label transfer from the expression profiles of the previously-published organoid atlas (Uzquiano et al., Cell, 2022) using Seurat's FindTransferAnchors and MapQuery functions. Cell-type labels were further refined in each merged or integrated dataset based on the joint cluster labels. Cell profiles (top 30 PCs) were embedded by UMAP. Clusters that had exceptionally low UMI counts were labeled as low-quality cells and removed from downstream analysis. Clusters that initially could only be described as cycling progenitors were subclustered by subsetting to only the cells from those clusters and rerunning PCA, k-nearest-neighbors graph construction, and clustering; expression of HOPX, EOMES, and SOX2 was used to identify these subclusters as aRG, oRG, or IP. In the case of the single-donor NSC-Chimeroids, one cluster which included both cells that expressed DLX2 and cells that expressed RELN was subclustered to distinguish immature interneurons and Cajal-Retzius cells.

### Pseudotime Analysis

Monocle3 version 1.1.3 (Cao et al., Nature, 2019; Qiu et al., Nat Methods, 2017; Trapnell et al., Nat Biotechnol, 2014) was applied to the scRNA-seq data from 3-mo, Mix 3 NSC-Chimeroids, using aRGs as the root cells.

Reproducibility and Comparison with Fetal Tissue and Cortical Organoids

Adjusted Mutual Information (AMI) scores were calculated between donor source and cell type calls for the cells within each batch of multi-donor Chimeroids and single-donor Chimeroids, using the aricode R package version 1.0.2 (Chiquet et al., 2022). Seurat's FindTransferAnchors and MapQuery functions were used as described in the Seurat 4.3 label-transfer tutorial to transfer labels from fetal cortex samples and cortical organoids onto Chimeroids and single-donor Chimeroids (and vice-versa).

Seurat's FindMarkers function, with no fold-change or significance threshold, was used to independently create a list of marker genes for each cell type in untreated NSC-Chimeroids, fetal cortical tissue or cortical organoids, ranked by significance as calculated via Wilcoxon rank-sum test, with the most significantly upregulated genes within a cell type at the top of the list, and the most significantly downregulated genes at the bottom. The improved rank-rank hypergeometric overlap test from the RRHO2 R package (version 1.0) was used to compare the marker gene lists from each NSC-Chimeroid cell type to those for each fetal and organoid marker list.

For each of 19 canonical cell type marker genes, the mean scaled expression across cells of each of 5 major cell types (aRG, ORG, IP, CFuPN, and CPN) was calculated in each of 5 datasets (Mix 1/Mix 2 NSC-Chimeroids, NPC-Chimeroids, single-donor Chimeroids, atlas organoids, and endogenous fetal cortex), in order to compare marker gene expression between datasets within comparable cell types. Pearson's correlation coefficient of those expression values was calculated between the endogenous fetal tissue and the corresponding cell types in each organoid and Chimeroid dataset, and also between the atlas organoids and each Chimeroid dataset.

### Cell Type Abundance, Donor Abundance, and Neighborhood Changes Between Treatment Conditions

A negative binomial mixed-effects model was used to test for differential cell type abundances between control Chimeroids and VPA or EtOH-treated Chimeroids. Specifically, the glmer.nb function from lme4 (version 1.1.33) was applied to a matrix of cell type counts per donor within each Chimeroid, using the log of the total number of cells per donor and Chimeroid as an offset, Chimeroid differentiation batch and donor Mix (Mix 1 or Mix 2) as fixed effects, and both donor identity (to account for general differences in cell type abundances between donors and similarities within donors across replicates) and individual Chimeroid (to account for similarities in cell type abundance between donors within a shared organoid) as random effects. Thus, a model formula of Cell Type~Treatment+offset (log (LibrarySize))+Batch+Mix+ (1|Chimeroid)+(1|Donor) was used. Significance of the effect of treatment on cell type abundance was calculated by comparing this model with a model without the fixed effect for treatment, but which was otherwise identical, via the anova function in the R stats package, version 4.2.2. Benjamini-Hochberg (BH) False Discovery Rates (FDR) were calculated based on the resulting p-values.

Additional negative binomial mixed-effects models were used to test whether the magnitude and/or direction of change in cell type in response to VPA or EtOH treatments were different between the different donors. Specifically, models similar to those described above were implemented, but with an additional random slope parameter allowing for the treatment effect to vary by donor. Thus, the anova function was used to compare a model with the formula: Cell Type~Treatment+offset (log (LibrarySize))+Batch+Mix+ (1|Chimeroid)+ (Treatment|Genotype) to an otherwise identical model where the (Treatment|Genotype) term with was replaced (1|Genotype). In cell types where the inclusion of the random slope term significantly improved the model (ORGs, IPs, and CPNs), post-hoc pairwise tests were performed between each pair of donors using the same mixed-effects models.

EdgeR version 3.40.2 (Chen et al., F1000Res, 2016; McCarthy et al., Nucleic Acids Res, 2012; Robinson et al., Bioinformatics, 2010) was used to test for changes in donor abundances in response to VPA or EtOH treatments. Specifically, a matrix of cell counts per donor within each Chimeroid was passed through the functions calcNormFactors (with method set to “TMM”), estimateDisp (with trend set to “none”), glmQLFit (with abundance.trend set to “FALSE”), and glmQLFTest, using a design formula with Chimeroid differentiation batch and donor Mix as additional covariates. Significance is reported as FDR, as calculated via BH.

The miloR R package version 1.6.0 (Dann et al., Nat Biotechnol, 2022) was used to assess differential abundance within overlapping cell neighborhoods ranging in size from 50-200 cells each. For each treatment (VPA and EtOH), the expression matrix was subset to include only the top 3,000 most variable genes within cells from control and treated Chimeroids, PCA was performed on that submatrix, and the result was passed into a Milo object for further analysis. miloR's buildGraph function was run with k=30 and d=30, followed by makeNhoods and calcNhoodDistance. Differential abundance (DA) testing was performed for treated vs control cells within each neighborhood using testNhoods with a design of ~Mix+Donor+Treatment. Significance is reported as a spatial FDR value calculated by miloR based on DA p-values and relationships between neighborhoods.

### Pseudobulk Profiles and Differential Expression Analysis

Pseudobulk profiles were generated both globally (wherein a pseudobulk profile comprises the UMIs from all cells from one donor within one chimeroid), and on a cell type-by-cell type basis (wherein a pseudobulk profile comprises the UMIs from all cells of one type from one donor within one chimeroid), by summing UMI counts for each gene and, where appropriate, calculating expression values as the log-Counts-Per-Million within each pseudobulk profile. Pseudobulk profiles comprising fewer than 20 cells were removed from downstream analyses.

Principal Component Analysis (PCA) was performed on highly variable genes (defined as genes where the mean expression plus the log of the variance-to-mean expression ratio is greater than 1) of the global pseudobulk expression matrices for the global and each cell type expression profiles. For each analysis, outliers (2-8 per expression matrix) were identified and removed by calculating the Euclidian distance between each sample and the centroid of all samples; outliers were defined as samples where this distance was more than 2.5 times the interquartile range above the upper quartile bound. PCA was re-run on the remaining samples for downstream analysis. The Euclidean distances between each pseudobulk profile across all PCs (the number of PCs in each case equaled the number of pseudobulked samples which passed quality and outlier filters) within each PCA were calculated using the dist( ) function from the stats R package. Analysis of Variance (ANOVA) was performed to assess whether distances between control and treated samples were significantly different between the five donor lines. When the ANOVA yielded a p-value of less than 0.05, post-hoc Tukey Tests were performed for pairwise comparisons between the donors.

### REFERENCES

Alfonso-Loeches, S., and Guerri, C. (2011). Molecular and behavioral aspects of the actions of alcohol on the adult and developing brain. Crit Rev Clin Lab Sci 48, 19-47
Arzua, T., Yan, Y., Jiang, C., Logan, S., Allison, R. L., Wells, C., Kumar, S. N., Schäfer, R., and Bai, X. (2020). Modeling alcohol-induced neurotoxicity using human induced pluripotent stem cell-derived three-dimensional cerebral organoids. Transl Psychiatry 10, 347PMC7553959.
Bjørk, M. H., Zoega, H., Leinonen, M. K., Cohen, J. M., Dreier, J. W., Furu, K., Gilhus, N. E., Gissler, M., Hálfdánarson, Ó., Igland, J., et al. (2022). Association of Prenatal Exposure to Antiseizure Medication With Risk of Autism and Intellectual Disability. JAMA Neurol 79, 672-681
Boulting, G. L., Kiskinis, E., Croft, G. F., Amoroso, M. W., Oakley, D. H., Wainger, B. J., Williams, D. J., Kahler, D. J., Yamaki, M., Davidow, L., et al. (2011). A functionally characterized test set of human induced pluripotent stem cells. Nat Biotechnol 29, 279-286PMC3229307.
Cahill, K. M., Huo, Z., Tseng, G. C., Logan, R. W., and Seney, M. L. (2018). Improved identification of concordant and discordant gene expression signatures using an updated rank-rank hypergeometric overlap approach. Sci Rep 8, 9588PMC6018631.
Cao, J., Spielmann, M., Qiu, X., Huang, X., Ibrahim, D. M., Hill, A. J., Zhang, F., Mundlos, S., Christiansen, L., Steemers, F. J., et al. (2019). The single-cell transcriptional landscape of mammalian organogenesis. Nature 566, 496-502PMC6434952.
Carpita, B., Migli, L., Chiarantini, I., Battaglini, S., Montalbano, C., Carmassi, C., Cremone, I. M., and Dell'Osso, L. (2022). Autism Spectrum Disorder and Fetal Alcohol Spectrum Disorder: A Literature Review. Brain Sci 12PMC9221419.
Cederquist, G. Y., Tchieu, J., Callahan, S. J., Ramnarine, K., Ryan, S., Zhang, C., Rittenhouse, C., Zeltner, N., Chung, S. Y., Zhou, T., et al. (2020). A Multiplex Human Pluripotent Stem Cell Platform Defines Molecular and Functional Subclasses of Autism-Related Genes. Cell Stem Cell 27, 35-49.e36PMC7376579.
Charness, M. E. (2022). Fetal Alcohol Spectrum Disorders: Awareness to Insight in Just 50 Years. Alcohol Res 42, 05PMC8885297.
Chen, Y., Lun, A. T., and Smyth, G. K. (2016). From reads to genes to pathways: differential expression analysis of RNA-Seq experiments using Rsubread and the edgeR quasi-likelihood pipeline. F1000Res 5, 1438PMC4934518.
Chiquet, J., Rigaill, G., Sundqvist, M., Dervieux, V., and Bersani, F. (2022). Efficient Computations of Standard Clustering Comparison Measures (CRAN). URL: cran.r-project.org/web/packages/aricode/index.html.
Christensen, J., Grønborg, T. K., Sørensen, M. J., Schendel, D., Parner, E. T., Pedersen, L. H., and Vestergaard, M. (2013). Prenatal valproate exposure and risk of autism spectrum disorders and childhood autism. JAMA 309, 1696-1703PMC4511955.
Church, G. M. (2005). The personal genome project. Mol Syst Biol 1, 2005.0030PMC1681452.
Cuomo, A. S. E., Seaton, D. D., McCarthy, D. J., Martinez, I., Bonder, M. J., Garcia-Bernardo, J., Amatya, S., Madrigal, P., Isaacson, A., Buettner, F., et al. (2020). Single-cell RNA-sequencing of differentiating iPS cells reveals dynamic genetic effects on gene expression. Nat Commun 11, 810PMC7010688.
Danecek, P., Bonfield, J. K., Liddle, J., Marshall, J., Ohan, V., Pollard, M. O., Whitwham, A., Keane, T., McCarthy, S. A., Davies, R. M., et al. (2021). Twelve years of SAMtools and BCFtools. Gigascience 10PMC7931819.
Dann, E., Henderson, N. C., Teichmann, S. A., Morgan, M. D., and Marioni, J. C. (2022). Differential abundance testing on single-cell data using k-nearest neighbor graphs. Nat Biotechnol 40, 245-253
Eberhart, J. K., and Parnell, S. E. (2016). The Genetics of Fetal Alcohol Spectrum Disorders. Alcohol Clin Exp Res 40, 1154-1165PMC5125635.
Ford, L. C., Jang, S., Chen, Z., Zhou, Y. H., Gallins, P. J., Wright, F. A., Chiu, W. A., and Rusyn, I. (2022). A Population-Based Human In Vitro Approach to Quantify Inter-Individual Variability in Responses to Chemical Mixtures. Toxics 10PMC9413237.
Germain, P. L., and Testa, G. (2017). Taming Human Genetic Variability: Transcriptomic Meta-Analysis Guides the Experimental Design and Interpretation of iPSC-Based Disease Modeling. Stem Cell Reports 8, 1784-1796PMC5470233.
Göttlicher, M., Minucci, S., Zhu, P., Krämer, O. H., Schimpf, A., Giavara, S., Sleeman, J. P., Lo Coco, F., Nervi, C., Pelicci, P. G., et al. (2001). Valproic acid defines a novel class of HDAC inhibitors inducing differentiation of transformed cells. Embo j 20, 6969-6978PMC125788.
Granato, A., and Dering, B. (2018). Alcohol and the Developing Brain: Why Neurons Die and How Survivors Change. Int J Mol Sci 19PMC6213645.
Jerber, J., Seaton, D. D., Cuomo, A. S. E., Kumasaka, N., Haldane, J., Steer, J., Patel, M., Pearce, D., Andersson, M., Bonder, M. J., et al. (2021). Population-scale single-cell RNA-seq profiling across dopaminergic neuron differentiation. Nat Genet 53, 304-312PMC7610897.
Kang, H. M., Subramaniam, M., Targ, S., Nguyen, M., Maliskova, L., McCarthy, E., Wan, E., Wong, S., Byrnes, L., Lanata, C. M., et al. (2018). Multiplexed droplet single-cell RNA-sequencing using natural genetic variation. Nat Biotechnol 36, 89-94PMC5784859.
Limone, F., Guerra San Juan, I., Mitchell, J. M., Smith, J. L. M., Raghunathan, K., Meyer, D., Ghosh, S. D., Couto, A., Klim, J. R., Joseph, B. J., et al. (2023). Efficient generation of lower induced motor neurons by coupling Ngn2 expression with developmental cues. Cell Rep 42, 111896PMC10117176.
Marguet, F., Brosolo, M., Friocourt, G., Sauvestre, F., Marcorelles, P., Lesueur, C., Marret, S., Gonzalez, B. J., and Laquerrière, A. (2022). Oligodendrocyte lineage is severely affected in human alcohol-exposed foetuses. Acta Neuropathol Commun 10, 74PMC9107108.
McCarthy, D. J., Chen, Y., and Smyth, G. K. (2012). Differential expression analysis of multifactor RNA-Seq experiments with respect to biological variation. Nucleic Acids Res 40, 4288-4297PMC3378882.
Meng, Q., Zhang, W., Wang, X., Jiao, C., Xu, S., Liu, C., Tang, B., and Chen, C. (2022). Human forebrain organoids reveal connections between valproic acid exposure and autism risk. Transl Psychiatry 12, 130PMC8964691.
Mitchell, J. M., Nemesh, J., Ghosh, S., Handsaker, R. E., Mello, C. J., Meyer, D., Raghunathan, K., de Rivera, H., Tegtmeyer, M., Hawes, D., et al. (2020). Mapping genetic effects on cellular phenotypes with “cell villages”. bioRxiv, 2020.2006.2029.174383. URL: biorxiv.org/content/early/2020/06/29/2020.06.29.174383.abstract. PMID: PMID. PMCID:
Neavin, D. R., Steinmann, A. M., Chiu, H. S., Daniszewski, M. S., Moutinho, C., Chan, C.-L., Tyebally, M., Gnanasambandapillai, V., Lam, C. E., Nguyen, U., et al. (2021). Village in a dish: a model system for population-scale hiPSC studies. bioRxiv
Nemesh, J., and Wysoker, A. (2023). Drop-seq. URL: github.com/broadinstitute/Drop-seq/.
Paulsen, B., Velasco, S., Kedaigle, A. J., Pigoni, M., Quadrato, G., Deo, A. J., Adiconis, X., Uzquiano, A., Sartore, R., Yang, S. M., et al. (2022). Autism genes converge on asynchronous development of shared neuron classes. Nature 602, 268-273PMC8852827.
Pizzo, L., Jensen, M., Polyak, A., Rosenfeld, J. A., Mannik, K., Krishnan, A., McCready, E., Pichon, O., Le Caignec, C., Van Dijck, A., et al. (2019). Rare variants in the genetic background modulate cognitive and developmental phenotypes in individuals carrying disease-associated variants. Genet Med 21, 816-825PMC6405313.
Polioudakis, D., de la Torre-Ubieta, L., Langerman, J., Elkins, A. G., Shi, X., Stein, J. L., Vuong, C. K., Nichterwitz, S., Gevorgian, M., Opland, C. K., et al. (2019). A Single-Cell Transcriptomic Atlas of Human Neocortical Development during Mid-gestation. Neuron 103, 785-801.e788PMC6831089.
Qiu, X., Hill, A., Packer, J., Lin, D., Ma, Y. A., and Trapnell, C. (2017). Single-cell mRNA quantification and differential analysis with Census. Nat Methods 14, 309-315PMC5330805.
R Core Team (2023). R: A language and environment for statistical computing. (Vienna, Austria: R Foundation for Statistical Computing). URL: R-project.org/.
Robinson, M. D., McCarthy, D. J., and Smyth, G. K. (2010). edgeR: a Bioconductor package for differential expression analysis of digital gene expression data. Bioinformatics 26, 139-140PMC2796818.
Schindelin, J., Arganda-Carreras, I., Frise, E., Kaynig, V., Longair, M., Pietzsch, T., Preibisch, S., Rueden, C., Saalfeld, S., Schmid, B., et al. (2012). Fiji: an open-source platform for biological-image analysis. Nat Methods 9, 676-682PMC3855844.
Sheridan, S. D., Theriault, K. M., Reis, S. A., Zhou, F., Madison, J. M., Daheron, L., Loring, J. F., and Haggarty, S. J. (2011). Epigenetic characterization of the FMR1 gene and aberrant neurodevelopment in human induced pluripotent stem cell models of fragile X syndrome. PLOS One 6, e26203PMC3192166.
Streissguth, A. P., and Dehaene, P. (1993). Fetal alcohol syndrome in twins of alcoholic mothers: concordance of diagnosis and IQ. Am J Med Genet 47, 857-861
Stuart, T., Butler, A., Hoffman, P., Hafemeister, C., Papalexi, E., Mauck, W. M., 3rd, Hao, Y., Stoeckius, M., Smibert, P., and Satija, R. (2019). Comprehensive Integration of Single-Cell Data. Cell 177, 1888-1902.e1821PMC6687398.
Sugathan, A., Biagioli, M., Golzio, C., Erdin, S., Blumenthal, I., Manavalan, P., Ragavendran, A., Brand, H., Lucente, D., Miles, J., et al. (2014). CHD8 regulates neurodevelopmental pathways associated with autism spectrum disorder in neural progenitors. Proc Natl Acad Sci USA 111, E4468-4477PMC4210312.
Sulik, K. K., Johnston, M. C., and Webb, M. A. (1981). Fetal alcohol syndrome: embryogenesis in a mouse model. Science 214, 936-938
Tegtmeyer, M., Arora, J., Asgari, S., Cimini, B. A., Peirent, E., Liyanage, D., Way, G., Weisbart, E., Nathan, A., Amariuta, T., et al. (2023). High-dimensional phenotyping to define the genetic basis of cellular morphology. bioRxiv
Thomson, J. A., Itskovitz-Eldor, J., Shapiro, S. S., Waknitz, M. A., Swiergiel, J. J., Marshall, V. S., and Jones, J. M. (1998). Embryonic stem cell lines derived from human blastocysts. Science 282, 1145-1147
Trapnell, C., Cacchiarelli, D., Grimsby, J., Pokharel, P., Li, S., Morse, M., Lennon, N. J., Livak, K. J., Mikkelsen, T. S., and Rinn, J. L. (2014). The dynamics and regulators of cell fate decisions are revealed by pseudotemporal ordering of single cells. Nat Biotechnol 32, 381-386PMC4122333.
Trevino, A. E., Müller, F., Andersen, J., Sundaram, L., Kathiria, A., Shcherbina, A., Farh, K., Chang, H. Y., Paşca, A. M., Kundaje, A., et al. (2021). Chromatin and gene-regulatory dynamics of the developing human cerebral cortex at single-cell resolution. Cell 184, 5053-5069.e5023
Uzquiano, A., Kedaigle, A. J., Pigoni, M., Paulsen, B., Adiconis, X., Kim, K., Faits, T., Nagaraja, S., Antón-Bolaños, N., Gerhardinger, C., et al. (2022). Proper acquisition of cell class identity in organoids allows definition of fate specification programs of the human cerebral cortex. Cell 185, 3770-3788.e3727PMC9990683.
Velasco, S., Kedaigle, A. J., Simmons, S. K., Nash, A., Rocha, M., Quadrato, G., Paulsen, B., Nguyen, L., Adiconis, X., Regev, A., et al. (2019). Individual brain organoids reproducibly form cell diversity of the human cerebral cortex. Nature 570, 523-527PMC6906116.
Villa, C. E., Cheroni, C., Dotter, C. P., López-Tóbon, A., Oliveira, B., Sacco, R., Yahya, A., Morandell, J., Gabriele, M., Tavakoli, M. R., et al. (2022). CHD8 haploinsufficiency links autism to transient alterations in excitatory and inhibitory trajectories. Cell Rep 39, 110615
Warren, C. R., and Cowan, C. A. (2018). Humanity in a Dish: Population Genetics with iPSCs. Trends Cell Biol 28, 46-57
Wells, M. F., Nemesh, J., Ghosh, S., Mitchell, J. M., Mello, C. J., Meyer, D., Raghunathan, K., Tegtmeyer, M., Hawes, D., Neumann, A., et al. (2021). Natural variation in gene expression and Zika virus susceptibility revealed by villages of neural progenitor cells. bioRxiv, 2021.2011.2008.467815. URL: biorxiv.org/content/early/2021/11/09/2021.11.08.467815.abstract. PMID: PMID. PMCID:
Wells, M. F., Nemesh, J., Ghosh, S., Mitchell, J. M., Salick, M. R., Mello, C. J., Meyer, D., Pietilainen, O., Piccioni, F., Guss, E. J., et al. (2023). Natural variation in gene expression and viral susceptibility revealed by neural progenitor cell villages. Cell Stem Cell 30, 312-332.e313
Williams, J. F., Smith, V. C., and ABUSE, t.C. O. S. (2015). Fetal Alcohol Spectrum Disorders. Pediatrics 136. URL: doi.org/10.1542/peds.2015-3113. PMID: PMID. PMCID:
Wozniak, J. R., Riley, E. P., and Charness, M. E. (2019). Clinical presentation, diagnosis, and management of fetal alcohol spectrum disorder. Lancet Neurol 18, 760-770PMC6995665.
Xuan Vinh, N., Epps, J., and Bailey, J. (2010). Information Theoretic Measures for Clusterings Comparison: Variants, Properties, Normalization and Correction for Chance Journal of Machine Learning Research 11, 2837-2854. URL: jmlr.org/papers/v11/vinh10a.html. PMID: PMID. PMCID:
Zheng, G. X., Terry, J. M., Belgrader, P., Ryvkin, P., Bent, Z. W., Wilson, R., Ziraldo, S. B., Wheeler, T. D., McDermott, G. P., Zhu, J., et al. (2017). Massively parallel digital transcriptional profiling of single cells. Nat Commun 8, 14049PMC5241818.

### Example 3—Chimeroid Protocol

### Reagents & Equipment:

### 1) Reagents

2-mercaptoethanol (ThermoFisher Scientific, #21985-023)·
B-27 Supplement, 50× (ThermoFisher Scientific, #17504044)
Bovine Serum Albumin (BSA) (Millipore-Sigma, #A3294-100G)
Chemically Defined Lipid Concentrate (ThermoFisher Scientific, #11905031)
Dibutyryl-cAMP (Stemcell Technologies, #100-0244)
DMEM/F-12 (ThermoFisher Scientific, #11330032)
DMSO (Sigma, #D2650)
Fungizone/Amphotericin B (ThermoFisher Scientific, #15290018)
Geltrex, LDEV-Free, hESC-Qualified, Reduced Growth Factor Basement Membrane Matrix (ThermoFisher Scientific, #A1413301)
Gentle Cell Dissociation Reagent (Stemcell Technologies, #100-0485)
Glasgow MEM, G-MEM (ThermoFisher Scientific, #11710035)
GlutaMAX Supplement (ThermoFisher Scientific, #35050061)
UltraPure DNase/RNase-free Distilled Water (ThermoFisher Scientific, #10977015)
Heparin (Sigma, #H3149-25KU)
Hyclone Fetal Bovine Serum (ThermoFisher Scientific, #SH30070.03HI)
IWR1 (Calbiochem/Millipore, #681669)
KnockOut Serum Replacement (ThermoFisher Scientific, #10828028)
Matrigel (Corning, #356234)
MEM Non-Essential Amino Acids Solution (ThermoFisher Scientific, #11140050)
mTeSR1 Stem Cell Medium (gibco, #85851/2)
mTeSR+Stem Cell Medium (gibco, #100-0274/5)
MycoAlert PLUS mycoplasma detection kit (Lonza, #LT07705)
N-2 Supplement, 100× (Life Technologies, #17502048)
Papain Dissociation System kit (Worthington, #LK003153)
PBS, phosphate buffered saline (ThermoFisher Scientific, #10010023)
Penicillin/Streptomycin Solution 100× (Corning, #30-002-CI)
ROCK inhibitor, Y-27632 (EMD Millipore, #SCM075)
SB 431542 (Stemcell Technologies, #100-1051)
Sodium Pyruvate (ThermoFisher Scientific, #11360070)
StemFlex Stem Cell Medium (gibco, #A3349401)
StemPro Accutase Cell Dissociation Reagent (ThermoFisher Scientific, #A1110501)
Steriflip-GP Sterile Centrifuge Tube Top Filter Unit (Millipore, #SCGP00525).
Trypan blue solution, 0.4% (ThermoFisher Scientific, #15250061)

### 2) Equipment

2 mL Aspirating Pipette (VWR, #357558)
5 mL Serological Pipette (VWR, #76184-748)
10 mL Serological “Wobble-Not” Pipette (VWR, #76420-068)
15 ml Conical Tubes, Polystyrene (VWR, #352097)
20 μL Sterile LTS Rainin Pipette Tips (Rainin, #30389226)
25 mL Serological Pipette (VWR, #76184-748)
35 μm cell strainer tube (ThermoFisher Scientific, #352235)
50 ml Conical Tubes, Polystyrene (VWR, #352070)
50 mL Serological Pipette (VWR, #75816-088)
60 mm TC-Treated Cell Culture Dish (Corning, #353002)
100 mm Ultra Low Culture Dish (Corning, #4615)
200 μL Sterile LTS Rainin Pipette Tips (Rainin, #30389240)
200 μL Wide-O Sterile LTS Rainin Pipette Tips (Rainin, #30389241)
1000 μL Sterile LTS Rainin Pipette Tips (Rainin, #30389213)
Cell lifter (Corning, #3008)
125 mL spinner flasks (Corning, #3152)
Centrifuges
CO₂ incubator
Countess II Automated Cell Counter (ThermoFisher Scientific, #AMQAX1000)
Countess Cell Counting Chamber Slides (ThermoFisher Scientific, #C10228)
Digital CO₂-resistant orbital shaker (ThermoFisher Scientific, #88881101)
1.5 mL DNA LoBind Tubes (VWR, #22431021)
Filter Bottles 0.22 μm 150 mL (VWR, #29442-938)
Filter Bottles 0.22 μm 250 mL (VWR, #28199-776)
Filter Bottles 0.22 μm 500 mL (VWR, #430769)
Filter Bottles 0.22 μm 1 L (VWR, #28199-812)·
Microscope, inverted
Digital CO₂-resistant magnetic spinner plate
Pipettor
Prime surface 96V plate, v-bottom (Sbio Japan, #MS9096VZ)
Rainin Micropipettes
Reagent reservoir (VWR, #89094680)

### Procedure:

### 1) Reagent Setup (as Previously Described by Velasco Protocol, Nature 2017):

Y-27632 (ROCK Inhibitor):
   Reconstitute 5 mg in 2.96 mL of sterile water to make a 5 mM stock solution.
   Store at −20° C. for up to 2 years in powder form, up to 1 year at −20° C. reconstituted. Light sensitive, avoid multiple freeze-thaw cycles.
SB431542 (Activin/BMP/TGF-β pathway inhibitor):
   Reconstitute 10 mg in 2.6 mL of DMSO to make a 10 mM stock solution.
   Store at −20° C. for up to 1 year in powder form, up to 3 months at −20° C. reconstituted. Light sensitive, avoid multiple freeze-thaw cycles.
IWR1 (Wnt Inhibitor):
   Reconstitute 10 mg in 814 μl of DMSO to make a 30 mM stock solution.
   Store at 4° C. for up to 7 years in powder form, up to 3 months at −20° C. reconstituted. Light sensitive, avoid multiple freeze-thaw cycles.
Heparin
   The concentration is measured in mg/ml, however the manufacturer reports quantity in units. Reconstitute with sterile PBS (10 mg/mL stock solution) and filter through a 0.22 μm filter.

### 2) Media Preparation (as Previously Described by Velasco Protocol):

CDM1 (d 0-d 18 before mixing +
aggregation day; quantities per 100 mL),

| Glasgow MEM, G-MEM | 77 | mL |
|---|---|---|
| KnockOut Serum Replacement | 20 mL - final 20% | |
| MEM Non-Essential Amino Acid Solution | 1 | mL |
| Sodium pyruvate | 1 | mL |
| 2-mercaptoethanol | 182 μL of 55 mM stock | |
| | (final 0.1 mM) | |
| Penicillin/Streptomycin Solution | 1 | mL |

After mixing all reagents, filter medium using a 0.22 μm filter bottle.

CDM2 (d 18-d 35, quantities per 100 mL):

| DMEM/F-12 | 100 | mL |
|---|---|---|
| GlutaMAX Supplement | 1 | mL |
| N-2 Supplement | 1 | mL |
| Chemically Defined Lipid Concentrate | 1 | mL |
| Fungizone | 100 μL of 250 μg/mL stock | |
| | (final 0.25 μg/mL) | |
| Penicillin/Streptomycin Solution | 1 | mL |

After mixing all reagents, filter medium using a 0.22 μm filter bottle.

CDM3 (d 35-d 70, quantities per 200 mL):

| DMEM/F-12 | 180 | mL |
|---|---|---|
| Hyclone Fetal Bovine Serum | 20 mL (final 10%) | |
| Chemically Defined Lipid Concentrate | 2 | mL |
| GlutaMAX Supplement | 2 | mL |
| N-2 Supplement | 2 | mL |
| Heparin | 100 uL of 10 mg/mL | |
| | (final 5 μg/mL) | |
| Fungizone | 200 μL of 250 μg/mL stock | |
| | (final 0.25 μg/mL) | |
| Penicillin/Streptomycin Solution | 2 | mL |
| Matrigel | 2 | mL |

After mixing reagents except Matrigel, filter medium using a 0.22 μm filter bottle. Add Matrigel after filtration while medium is cold.

CDM4 (d 70 onwards, quantities per 200 mL):

| DMEM/F-12 | 170 | mL |
|---|---|---|
| Hyclone Fetal Bovine Serum | 20 mL (final 10%) | |
| Chemically Defined Lipid Concentrate | 2 | mL |
| GlutaMAX Supplement | 2 | mL |
| N-2 Supplement | 2 | mL |
| B-27 Supplement | 4 | mL |
| Heparin | 100 uL of 10 mg/mL stock | |
| | (5 μg/mL) | |
| Fungizone | 200 μL of 250 μg/L stock | |
| | (final 0.25 μg/mL) | |
| Penicillin/Streptomycin Solution | 2 | mL |
| Matrigel | 4 | mL |

After mixing reagents except Matrigel, filter medium using a 0.22 μm filter bottle. Add Matrigel after filtration while the media is cold.

### 3) Generation of Organoids (as Previously Described by Velasco Protocol, Nature 2017) and Chimeroids:

Maintenance of human pluripotent stem cells (hPSCs):
   Maintain feeder-free hPSCs on TC-treated cell culture dishes pre-coated with 1:100 Geltrex/DMEM-F12 in mTeSR+, mTeSR1 or StemFlex medium, passaged with non-enzymatic reagents (e.g. Gentle or ReLeSR) at 37° C. and 5% CO₂.
      Avoid single-cell dissociation during passaging.
      Avoid surpassing 50 passages. Routinely confirm karyotype stability and test for mycoplasma.
         Detailed protocol for thawing, maintenance, and passaging of hPSCs can be found in Quadrato et al., 2017.
Generation and maintenance of cortical brain organoids until Chimeroid generation (NSC-Ch, div15-18 or NPC-Ch, div23-25)-Brain Cortical Organoids (Velasco et al., 2019)
   d0: Enzymatically dissociate 75-85% confluent human iPSC or ESCs and re-aggregate in ultra-low attachment v-bottom 96 well plates at a density of 9,000 cells per well.
      Note modification from Velasco protocol: At d0 protocol deviates from Velasco; seeding and generation of EBs (Embryoid bodies) is done in the preferred PSC culture media (according with the media in which PSCs are cultured)+20 μM Rock Inhibitor until 20-24 hrs following seeding.
   d1: Aspirate 80 μL per well of aggregation media and replace with 100 μL CDM1 with appropriate dorsal telencephalon patterning factors previously described in Velasco et al, 2017.
   d3: Add additional 100 μL CDM1 with appropriate dorsal patterning factors. (Do not aspirate media on this day).
   d6-d15: Aspirate 80 μL per well of aggregation media and replace with 100 μL CDM1 with appropriate dorsal patterning factors described in Velasco protocol.
      CRITICAL: before adding media, gently agitate (up-down of 20 μL approximately) the media by using a multi-channel pipette. This step helps detach and wash dead cells that might surround the EBs.
   d18: Using Rainin Wide-O 200 μL tips, transfer organoids from 96 well aggregation plate to 100 mm ultra-low attachment culture dish with 15 mL CDM2 per dish; agitate at 90 rpm.
      Note: If EBs become too big, can be transferred to 10 cm low attachment plates earlier (total volume 15 mL) from DIV9 to avoid stress. Important to follow patterning protocol until DIV15-18 (Velasco protocol).
   d21-35: Replace CDM2 every three days.
      Detailed protocol for generation and maintenance of Velasco dorsally patterned forebrain organoids can be found in Velasco et al., 2019.
Organoid Dissociation and Re-Aggregation to form Chimeroid EBs
   Note: organoids can be dissociated and re-aggregated at either d15-d18 to generate NSC-Chimeroids or d23-d25 to generate NPC-Chimeroids.
      NSCs (Neural Stem Cells)
      NPCs (Neural Progenitor Cells)
   Step 1: Prepare papain enzyme and albumin inhibitor following
   Worthington Papain Dissociation Kit instructions.
   Step 2: Prepare Chimeroid seeding medium (CH.SM)
      CH.SM: CDM1+20 μM Rock inhibitor (add 2 μM/mL-5 mM stock concentration).
      CH.SM is used for resuspending the cells after centrifugation and for seeding.
         For resuspension: 500 μL per cell line for NSC-Chimeroids; 1 mL per cell line for NPC-Chimeroids.
         For seeding: 10 mL/96-well plate.
   Step 3: Select organoids properly patterned dorsal forebrain organoids with the appropriate morphology.
      NSC-Chimeroids: select roughly 20-25 organoids per condition.
      NPC-Chimeroids: select roughly 12-18 organoids per condition.
      NOTE: number of organoids selected dependent on the size of the organoids, which relies on the specific features of the PSC lines that are used.
   Step 4: Transfer organoids to DNA low-bind tubes (1× per condition) and discard remaining media; replace with 500-1000 μL warmed papain solution and mince with a p1000 pipette (10×).
      NOTE: the volume used for papain dissociation relies on the number of organoids and size.
      NSC-Chimeroids: 500 μL warmed papain solution.
      NPC-Chimeroids: 1000 μL warmed papain solution.
   Step 5: Incubate at 37° C. and 5% CO₂ while shaking at 90 rpm for 25 minutes.
   Step 6: Further mince with a p1000 pipette (15×) and incubate for an additional 5 minutes.
   Step 7: Perform final mincing (10-20×) and transfer the resulting single-cell suspension to a 15 mL Falcon tube that contains the proper volume of inhibitor solution (IS, check manufacture instructions).
      NOTE: IS contains Earle's Balanced Salt Solution and albumin inhibitor in 1:0.6 ratio. The total volume depends on the initial papain used during the dissociation process (follow manufacturer instructions).
   Step 8: Centrifuge the mixture at 300 g for 5 minutes. Aspirate the resulting supernatant and resuspend cell pellet in 500 μL seeding media (CH.SM) and filter the solution using a 35 μm filter top tube.
      NOTE: the volume of seeding media relies on the expected concentration of cells/mL. 500 μL seeding media for 1 to 9 million cells/mL.
   Step 9: In a separate Eppendorf tube, combine 10 μL of the resuspended cell solution with 10 μL of Trypan blue and seed the resulting mixture into Countess chamber slides. Count cells using Countess II.
   Step 10: Seed 15-20k (for d15-18 mixes) or 50k (for d23-25 mixes) cells per well in a ultra-low attachment v-bottom 96 well plate and allow to aggregate for 24 hours.
Maintenance of Chimeric Organoids:
   d1 after mixing (AM): 24 hours following seeding embryo bodies (EBs) have formed (characterized by uniform distribution of live cells across the aggregate with a clearly-defined outer boundary; minimal cell death is normal). Replace 80 μL of media with 100 μL of CDM2.
   d2AM: Using Rainin Wide-O 200 μL tips, transfer EBs to a 100 mm ultra-low attachment culture dish with 15 mL CDM2 per dish; agitate at 90 rpm.
   d5AM-div35: Change 15 mL of CDM2 every 3 days following Velasco protocol.
   div35: Transfer chimeric organoids to a 125 mL spinner flask with 100 mL CDM3; set to rotate magnetic stirrer arm at 57 rpm.
   div35-div70: Replace 100 mL CDM3 once weekly following Velasco protocol.
   div70. Replace CDM3 with CDM4.
   >div70: Replace 100 mL CDM4 once weekly following Velasco protocol.

### Example 4—NSC/NPC Biobanking Protocol

### Reagents & Equipment

### 1) Reagents

2-mercaptoethanol (ThermoFisher Scientific, #21985-023)
B-27 Supplement, 50× (ThermoFisher Scientific, #17504044)
Bovine Serum Albumin (BSA) (Millipore-Sigma, #A3294-100G)
Chemically Defined Lipid Concentrate (ThermoFisher Scientific, #11905031)
CryoStor Freeze Media CS10 (BioLife Solutions, #210210)
DMEM/F-12 (ThermoFisher Scientific, #11330032)
DMSO (Sigma, #D2650)
Fungizone/Amphotericin B (ThermoFisher Scientific, #15290018)
Geltrex, LDEV-Free, hESC-Qualified, Reduced Growth Factor Basement
Membrane Matrix (ThermoFisher Scientific, #A1413301) o Gentle Cell
Dissociation Reagent (Stemcell Technologies, #100-0485)
Glasgow MEM, G-MEM (ThermoFisher Scientific, #11710035)
GlutaMAX Supplement (ThermoFisher Scientific, #35050061)
UltraPure DNase/RNase-free Distilled Water (ThermoFisher Scientific, #10977015)
Heparin (Sigma, #H3149-25KU)
Hyclone Fetal Bovine Serum (ThermoFisher Scientific, #SH30070.03HI)
IWR1 (Calbiochem/Millipore, #681669)
KnockOut Serum Replacement (ThermoFisher Scientific, #10828028)
Matrigel (Corning, #356234)
MEM Non-Essential Amino Acids Solution (ThermoFisher Scientific, #11140050)
mTeSR1 Stem Cell Medium (gibco, #85851/2)
mTeSR+Stem Cell Medium (gibco, #100-0274/5)
MycoAlert PLUS mycoplasma detection kit (Lonza, #LT07705)
N-2 Supplement, 100× (Life Technologies, #17502048)
Papain Dissociation System kit (Worthington, #LK003153)
PBS, phosphate buffered saline (ThermoFisher Scientific, #10010023)
Penicillin/Streptomycin Solution 100× (Corning, #30-002-CI)
ROCK inhibitor, Y-27632 (EMD Millipore, #SCM075)
SB 431542 (Stemcell Technologies, #100-1051)
Sodium Pyruvate (ThermoFisher Scientific, #11360070)
StemFlex Stem Cell Medium (gibco, #A3349401)
StemPro Accutase Cell Dissociation Reagent (ThermoFisher Scientific, #A1110501)
Steriflip-GP Sterile Centrifuge Tube Top Filter Unit (Millipore, #SCGP00525)
Trypan blue solution, 0.4% (ThermoFisher Scientific, #15250061)

### 2) Equipment

2 mL Aspirating Pipette (VWR, #357558)
5 mL Serological Pipette (VWR, #76184-748)
10 mL Serological “Wobble-Not” Pipette (VWR, #76420-068)
15 mL Conical Tubes, Polystyrene (VWR, #352097)
20 μL Sterile LTS Rainin Pipette Tips (Rainin, #30389226)
25 mL Serological Pipette (VWR, #76184-748)
35 μm cell strainer tube (ThermoFisher Scientific, #352235)
50 ml Conical Tubes, Polystyrene (VWR, #352070) 0
50 mL Serological Pipette (VWR, #75816-088)
60 mm TC-Treated Cell Culture Dish (Corning, #353002)
100 mm Ultra Low Culture Dish (Corning, #4615)
200 μL Sterile LTS Rainin Pipette Tips (Rainin, #30389240)
200 μL Wide-O Sterile LTS Rainin Pipette Tips (Rainin, #30389241)
1000 μL Sterile LTS Rainin Pipette Tips (Rainin, #30389213)
Cell lifter (Corning, #3008)
125 mL spinner flasks (Corning, #3152)
Centrifuges
CO₂ incubator
Countess II Automated Cell Counter (ThermoFisher Scientific, #AMQAX1000)
Countess Cell Counting Chamber Slides (ThermoFisher Scientific, #C10228)
Nunc Cryotube Cryogenic Vials (Sigma, #V7634)
Digital CO₂-resistant orbital shaker (ThermoFisher Scientific, #88881101)
1.5 mL DNA LoBind Tubes (VWR, #22431021)
Filter Bottles 0.22 μm 150 mL (VWR, #29442-938)
Filter Bottles 0.22 μm 250 mL (VWR, #28199-776)
Filter Bottles 0.22 μm 500 mL (VWR, #430769)
Filter Bottles 0.22 μm 1 L (VWR, #28199-812)
Microscope, inverted
Nalgene Freezing Container “Mr. Frosty” (Sigma, #C1562-1EA)·
Digital CO₂-resistant magnetic spinner plate
Pipettor
Prime surface 96V plate, v-bottom (Sbio Japan, #MS9096VZ)
Rainin Micropipettes
Reagent reservoir (VWR, #89094680)

### Procedure:

### 1) Reagent Setup (as Previously Described by Velasco Protocol, Nature 2017):

Y-27632 (ROCK Inhibitor):
   Reconstitute 5 mg in 2.96 mL of sterile water to make a 5 mM stock solution.
   Store at −20° C. for up to 2 years in powder form, up to 1 year at −20° C. reconstituted. Light sensitive, avoid multiple freeze-thaw cycles.
SB431542 (Activin/BMP/TGF-β pathway inhibitor):
   Reconstitute 10 mg in 2.6 mL of DMSO to make a 10 mM stock solution.
   Store at −20° C. for up to 1 year in powder form, up to 3 months at −20° C. reconstituted. Light sensitive, avoid multiple freeze-thaw cycles.
IWR1 (Wnt Inhibitor):
   Reconstitute 10 mg in 814 μl of DMSO to make a 30 mM stock solution.
   Store at 4° C. for up to 7 years in powder form, up to 3 months at −20° C. reconstituted. Light sensitive, avoid multiple freeze-thaw cycles.
Heparin
   The concentration is measured in mg/ml, however the manufacturer reports quantity in units. Reconstitute with sterile PBS (10 mg/mL stock solution) and filter through a 0.22 μm filter.

### 2) Media Preparation (as Previously Described by Velasco Protocol):

CDM1 (d 0-d 18 before mixing +
aggregation day; quantities per 100 mL),

| Glasgow MEM, G-MEM | 77 | mL |
|---|---|---|
| KnockOut Serum Replacement | 20 mL - final 20% | |
| MEM Non-Essential Amino Acid Solution | 1 | mL |
| Sodium pyruvate | 1 | mL |
| 2-mercaptoethanol | 182 μL of 55 mM stock | |
| | (final 0.1 mM) | |
| Penicillin/Streptomycin Solution | 1 | mL |

After mixing all reagents, filter medium using a 0.22 μm filter bottle.

CDM2 (d 18-d 35, quantities per 100 mL):

| DMEM/F-12 | 100 | mL |
|---|---|---|
| GlutaMAX Supplement | 1 | mL |
| N-2 Supplement | 1 | mL |
| Chemically Defined Lipid Concentrate | 1 | mL |
| Fungizone | 100 μL of 250 μg/mL stock | |
| | (final 0.25 μg/mL) | |
| Penicillin/Streptomycin Solution | 1 | mL |

After mixing all reagents, filter medium using a 0.22 μm filter bottle.

CDM3 (d 35-d 70, quantities per 200 mL):

| DMEM/F-12 | 180 | mL |
|---|---|---|
| Hyclone Fetal Bovine Serum | 20 mL (final 10%) | |
| Chemically Defined Lipid Concentrate | 2 | mL |
| GlutaMAX Supplement | 2 | mL |
| N-2 Supplement | 2 | mL |
| Heparin | 100 uL of 10 mg/mL | |
| | (final 5 μg/mL) | |
| Fungizone | 200 μL of 250 μg/mL stock | |
| | (final 0.25 μg/mL) | |
| Penicillin/Streptomycin Solution | 2 | mL |
| Matrigel | 2 | mL |

After mixing reagents except Matrigel, filter medium using a 0.22 μm filter bottle. Add Matrigel after filtration while medium is cold.

CDM4 (d 70 onwards, quantities per 200 mL):

| DMEM/F-12 | 170 | mL |
|---|---|---|
| Hyclone Fetal Bovine Serum | 20 mL (final 10%) | |
| Chemically Defined Lipid Concentrate | 2 | mL |
| GlutaMAX Supplement | 2 | mL |
| N-2 Supplement | 2 | mL |
| B-27 Supplement | 4 | mL |
| Heparin | 100 uL of 10 mg/mL stock | |
| | (5 μg/mL) | |
| Fungizone | 200 μL of 250 μg/L stock | |
| | (final 0.25 μg/mL) | |
| Penicillin/Streptomycin Solution | 2 | mL |
| Matrigel | 4 | mL |

*After mixing reagents except Matrigel, filter medium using a 0.22 μm filter bottle. Add Matrigel after filtration while the media is cold.

### 3) Generation of Organoids (as Previously Described by Velasco Protocol, Nature 2017) and Chimeroids:

Maintenance of human pluripotent stem cells (hPSCs):
   Maintain feeder-free hPSCs on TC-treated cell culture dishes pre-coated with 1:100 Geltrex/DMEM-F12 in mTeSR+, mTeSR1 or StemFlex medium, passaged with non-enzymatic reagents (e.g., Gentle or ReLeSR) at 37° C. and 5% CO₂.
      Avoid single-cell dissociation during passaging.
      Avoid surpassing 50 passages. Routinely confirm karyotype stability and test for mycoplasma.
   Detailed protocol for thawing, maintenance, and passaging of hPSCs can be found in Quadrato et al., 2017.
Generation and maintenance of cortical brain organoids pre NSC/NPC biobanking (Thw-NSC-Chimeroids, div15-18 or Thw-NPC-Chimeroids, div23-25) Brain Cortical organoids (Velasco et al., 2019)
   d0: Enzymatically dissociate 75-85% confluent human iPSC or ESCs and re-aggregate in ultra-low attachment v-bottom 96 well plates at a density of 9,000 cells per well.
      Note modification from Velasco protocol: At d0 protocol deviates from Velasco; seeding and generation of EBs (Embryoid bodies) is done in the preferred PSC culture media (according with the media in which PSCs are cultured)+20 μM Rock Inhibitor until 20-24 hrs following seeding.
   d1: Aspirate 80 μL per well of aggregation media and replace with 100 μL CDM1 with appropriate dorsal telencephalon patterning factors previously described in Velasco et al, 2017.
   d3: Add additional 100 μL CDM1 with appropriate dorsal patterning factors. (Do not aspirate media on this day).
   d6-d15: Aspirate 80 μL per well of aggregation media and replace with 100 μL CDM1 with appropriate dorsal patterning factors described in Velasco protocol.
   d18: Using Rainin Wide-O 200 μL tips, transfer organoids from 96 well aggregation plate to 100 mm ultra-low attachment culture dish with 15 mL CDM2 per dish; agitate at 90 rpm.
   d21-35: Replace CDM2 every three days.
Organoid Dissociation and Biobanking
   Note: organoids can be dissociated and frozen at either d15-d18 to generate NSC-Chimeroids or d23-d25 to generate NPC-Chimeroids.
      NSCs (Neural Stem Cells)
      NPCs (Neural Progenitor Cells)
   Step 1: Prepare papain enzyme and albumin inhibitor following Worthington Papain Dissociation Kit instructions.
   Step 2: Prepare freezing medium and Mr. Frosty-ensure CryoStor and Mr. Frosty are pre-chilled at 4° C.
   Step 3: Select organoids properly patterned dorsal forebrain organoids with the appropriate morphology.
      NSC-Chimeroids: select roughly 20-25 organoids per condition (to obtain one entire plate of organoids).
      NPC-Chimeroids: select roughly 12-18 organoids per condition (to obtain one entire plate of organoids).
      NOTE: number of organoids selected dependent on the size of the organoids, which relies on the specific features of the PSC lines that are used.
   Step 4: Transfer organoids to DNA low-bind tubes (1× per condition) and discard remaining media; replace with 500-1000 μL warmed papain solution and mince with a p1000 pipette (10×).
      NOTE: the volume used for papain dissociation relies on the number of organoids and size.
      NSC-Chimeroids: 500 μL warmed papain solution.
      NPC-Chimeroids: 1000 μL warmed papain solution.
   Step 5: Incubate at 37° C. and 5% CO₂ while shaking at 90 rpm for 25 minutes.
   Step 6: Further mince with a p1000 pipette (15×) and incubate for an additional 5 minutes.
   Step 7: Perform final mincing (10-20×) and transfer the resulting single-cell suspension to a 15 mL Falcon tube that contains the proper volume of inhibitor solution (IS, check manufacture instructions).
      NOTE: IS contains Earle's Balanced Salt Solution and albumin inhibitor in 1:0.6 ratio. The total volume depends on the initial papain used during the dissociation process (follow manufacturer instructions).
   Step 8: Centrifuge the mixture at 300 g for 5 minutes. Aspirate the resulting supernatant and resuspend cell pellet in 250 μL chilled (4° C.) CryoStor Freezing Medium and filter the solution using a 35 μm filter top tube.
   Step 9: Transfer resuspended cell mixture to Cryo Vials and place immediately in chilled Mr. Frosty at 4° C. for 10 minutes.
   Step 10: Transfer Mr. Frosty with Cryo Vials to −80° C. and incubate overnight.
   Step 11: Transfer CryoVials to liquid nitrogen storage until thawing.
NSC/NPC Thawing and Re-Aggregation (Thw-embryoid body formation)
   Step 1: Prepare Chimeroid Thawing/Seeding Medium (CH.ThwM) CH.ThwM: CDM1+20 μM Rock inhibitor (add 2 μM/mL-5 mM stock concentration).
      CH.ThwM is used for washing the cells after thawing, resuspension and for seeding.
         For washing: 5 mL per Cryo Vial.
         For resuspension: 1 mL per Cryo Vial.
         For seeding: 10 mL/96-well plate.
   Step 2: Gently thaw Cryo Vials in a 37° C. water bath by swirling tubes in water.
   Step 3: When fully thawed, transfer 250 μL CryoStor suspension mixture to a 15 mL Falcon tube with 5 mL of CH.ThwM and centrifuge at 200 g for 4 min.
   Step 4: Aspirate supernatant; resuspend in 1 mL CH.ThwM.
   Step 5: In a separate Eppendorf tube, combine 10 μL of the resuspended cell solution with 10 μL of Trypan blue and seed the resulting mixture into Countess chamber slides. Count using Countess II.
   Step 6: Seed 30-50k (for d15-18 NSC-Chimeroids) or 60-80k (for d23-25 NPC-Chimeroids) cells per well in an ultra-low attachment v-bottom 96 well plate and allow to aggregate for 24-48 hours.
Maintenance of Thw-Chimeroids:
   d1 after mixing: 24 hours following seeding embryo bodies (EBs) have formed (characterized by uniform distribution of live cells across the aggregate with a clearly-defined outer boundary; minimal cell death is normal). Add 100 μL of CDM2 without disturbing CH.ThwM.
   d2-3AM: Using Rainin Wide-O 200 μL tips, transfer EBs to a 100 mm ultra-low attachment culture dish with 15 mL CDM2 per dish; agitate at 90 rpm.
      NOTE: Thw-Chimeroids may take longer than 24 h to fully aggregate; monitor condition on d2-3 after mixing and transfer to 10 cm when fully aggregated.
   d5AM-div35: Change 15 mL of CDM2 every 3 days following Velasco protocol.
   div35: Transfer chimeric organoids to a 125 mL spinner flask with 100 mL CDM3; set to rotate magnetic stirrer arm at 57 rpm.
   div35-div70: Replace 100 mL CDM3 once weekly following Velasco protocol.
   div70. Replace CDM3 with CDM4.
   >div70: Replace 100 mL CDM4 once weekly following Velasco protocol.

What is claimed is:

1. A method for making a chimeric organoid containing cells of chimeric origin, comprising:
obtaining at least one embryoid body from each of at least two chimeric origins;
producing a patterned organoid from each of the embryoid bodies;
dissociating at least a portion of the cells from at least two of the patterned organoids to produce dissociated cells from each of the patterned organoids;
mixing at least a portion of the dissociated cells produced in step (c); and
culturing and expanding the mixed cells of step (d) under conditions and for a length of time sufficient to form a fully-formed brain organoid containing cells of chimeric origin.

2. The method of claim 1, wherein the cells of chimeric origin are derived from multiple individuals of the same species.

3. The method of claim 1, wherein the cells of chimeric origin are derived from one or more individuals from at least two different species.

4. (canceled)

5. (canceled)

6. The method of claim 1, wherein the cells of chimeric origin are derived from distinct areas of the brain at least two discrete regions of the brain selected from the group consisting of the pallial, subpallial, cerebral cortex, or ventral forebrain regions of the brain.

7. (canceled)

8. The method of claim 1, wherein the cells of chimeric origin are derived from one or more distinct regions of the brain selected from the group consisting of pallial, subpallial, cerebral cortex, or ventral forebrain regions of the brain and from one or more distinct regions outside the brain selected from the group consisting of the microglia, retina, or the vasculature.

9. (canceled)

10. The method of claim 1, wherein the cells of chimeric origin are derived from two or more embryological origins.

11. The method of claim 10, wherein the cells of chimeric origin originate from at least two of endothelial cells, cardiac cells, and gut cells.

12. The method of claim 1, wherein step a) comprises obtaining a stem cell from at least two human donors.

13. The method of claim 1, wherein the mixed cells further comprise one or more sensory cells.

14. The method of claim 13, wherein the sensory cells comprise cells derived from a retina.

15. A chimeric organoid comprising cells of chimeric origin.

16. A chimeric organoid comprising cells of chimeric origin produced by the method of claim 1.

17. The chimeric organoid of claim 15, wherein the cells of chimeric origin are derived from multiple individuals of the same species.

18. The chimeric organoid of claim 15, wherein the cells of chimeric origin are derived from one or more individuals from at least two different species.

19. (canceled)

20. (canceled)

21. The chimeric organoid of claim 15, wherein the cells of chimeric origin are derived from distinct areas of the brain at least two discrete regions of the brain selected from the group consisting of the pallial, subpallial, cerebral cortex, or ventral forebrain regions of the brain.

22. (canceled)

23. The chimeric organoid of claim 15, wherein the cells of chimeric origin are derived from one or more distinct regions of the brain selected from the group consisting of pallial, subpallial, cerebral cortex, or ventral forebrain regions of the brain and from one or more distinct regions outside the brain selected from the group consisting of the microglia, retina, or the vasculature.

24. (canceled)

25. The chimeric organoid of claim 15, wherein the cells of chimeric origin are derived from two or more embryological origins.

26. The chimeric organoid of claim 25, wherein the cells of chimeric origin originate from at least two of endothelial cells, cardiac cells, and gut cells.

27. The chimeric organoid of claim 15, wherein the cells are obtained from at least two human donors.

28. A method of biobanking comprising:
generating a population of dorsal forebrain organoids;
dissociating the generated dorsal forebrain organoids at d15-d18 or d23-d25; and
freezing the dissociated dorsal forebrain organoids comprising:
transferring the dissociated dorsal forebrain organoids to a warmed solution containing papain enzyme,
incubating the solution containing the dissociated dorsal forebrain organoids;
mincing the solution to produce a single-cell suspension;
mixing the single-cell suspension with an inhibitor solution;
centrifuging the mixture and resuspending the resulting cell pellet in freezing medium; and
freezing the resuspended mixture.


SEQUENCE LISTING (1 sequence)

SEQ ID NO: 1 - RNA, 20 nt, synthetic construct
  source 1..20: mol_type = other RNA
ttcttactgtgtacccgggc